SEQUENCE LISTING (138 sequences)

SEQ ID NO: 1 - DNA, 912 bp, Artificial
  Synthetic gene
atgtcccaagctttcaccgaatctgttaagacttctttgggtccaaatgctactccaagagctaaaaagttgattgcctctttggttcaacacgttcatgatttcgctagagaaaaccatttgactaccgaagattggttgtggggtgttgatttcattaacagaattggtcaaatgtccgactccagaagaaacgaaggtattttggtttgcgatatcatcggtttggaaaccttggttgatgctttgactaacgaatccgaacaatctaaccatacctcctctgctattttgggtcctttttacttgccagattctccagtttatccaaacggtggttctatcgttcaaaaggctattccaactgatgttaagtgcttcgttagaggtaaggttactgatactgaaggtaaaccattgggtggtgctcaattggaagtttggcaatgtaattctgctggtttctactctcaacaagctgatcatgatggtccagaattcaatttgagaggtactttcattaccgacgacgaaggtaattactccttcgaatgtttaagaccaacctcctatccaattccatacgatggtcctgctggtgatttgttgaaaatcatggatagacatccaaacagaccatcccatattcattggagagtttctcatccaggttaccatactttgatcacccaaatctatgatgctgaatgtccatacaccaacaacgattctgtttacgctgttaaggatgacatcatcgttcacttcgaaaaggttgataacaaggataaggatttggtcggtaaggtcgaatacaagttggattacgatatttccttggccaccgaatcctctattcaagaagctagagctgctgctaaagctagacaagatgctgaaatcaagttgtaa

SEQ ID NO: 2 - DNA, 594 bp, Artificial
  Synthetic gene
atgaagttgatcatcggtatgactggtgctacaggtgctccattgggtgttgctttgttgcaagctttgagagatatgccagaagttgaaacccatttggttatgtctaaatgggctaagaccaccattgaattggaaactccatggactgctagagaagttgctgctttggctgatttttctcattctccagctgatcaagctgctactatttcttctggttctttcagaactgatggtatgatcgttattccatgctctatgaaaaccttggctggtattagagctggttatgctgaaggtttggttggtagagctgctgatgttgttttgaaagaaggtagaaagttggtcttggtcccaagagaaatgccattgtctactatccatttggaaaacatgttggccttgtctagaatgggtgtagctatggttccaccaatgccagcttattacaatcatccagaaaccgttgatgacatcaccaaccatatagttaccagagttttggaccaattcggtttggattatcacaaagctagaagatggaacggtttgagaactgctgaacaattcgctcaagaaattgaatga

SEQ ID NO: 3 - DNA, 1509 bp, Artificial
  Synthetic gene
atgaccgccccaatccaagatttgagagatgctattgctttgttacaacaacacgacaatcaatacttggaaaccgatcatccagttgatccaaatgctgaattggctggtgtttacagacatattggtgctggtggtactgtaaaaagaccaactagaattggtccagccatgatgttcaacaacattaagggttatccacactccagaatcttggttggtatgcatgcttctagacaaagagcagctttgttgttgggttgtgaagcttctcaattggctttggaagttggtaaagctgttaagaaaccagttgctccagttgttgttccagcttcttctgctccatgtcaagaacaaattttcttggctgatgatccagacttcgatttgagaactttgttgccagctcataccaacactccaattgatgctggtccatttttttgtttgggtttggctttagcttctgatcctgttgatgcttctttgaccgatgttaccattcatagattgtgcgttcaaggtagagatgaattgtctatgtttttggctgccggtagacatatcgaagtttttagacaaaaagctgaagctgctggtaagccattgccaattactattaacatgggtttagatccagccatctacattggtgcttgttttgaagctccaactactccatttggttacaacgaattgggtgttgctggtgctttgagacaaagaccagttgaattggttcaaggtgtttctgttccagaaaaggctattgctagagccgaaatagttatcgaaggtgaattattgccaggtgtcagagttagagaagatcaacatacaaattccggtcatgctatgccagaatttccaggttattgtggtggtgctaatccatctttgccagttattaaggttaaggccgttaccatgagaaacaacgctattttacaaactttggtcggtccaggtgaagaacatacaactttggctggtttgccaaccgaagcttctatttggaatgctgttgaagctgcaattccaggtttcttgcaaaatgtttatgctcatacagctggtggtggtaagttcttgggtatattgcaagtcaagaaaagacaaccagctgacgaaggtagacaaggtcaagctgctttattagctttggctacttactccgaattgaagaatatcatcttggtcgatgaagatgttgatatcttcgattccgatgatattttgtgggctatgactactagaatgcaaggtgatgtttccattactaccattccaggtattagaggtcaccaattagatccatctcaaaccccagaatactccccatcaattagaggtaatggtatctcctgtaagaccattttcgattgcactgttccatgggctttgaagtctcattttgaaagagcaccatttgctgacgttgatcctagaccttttgctccagaatatttcgctagattggaaaagaatcaaggttccgctaagtaa

SEQ ID NO: 4 - DNA, 237 bp, Artificial
  Synthetic gene
atgatctgtccaagatgcgccgacgaaaaaattgaagttatggctacttctccagttaagggtgtttggactgtttatcaatgtcaacactgcttgtacacttggagagatactgaaccattgagaagaacctctagagaacattaccctgaagctttcagaatgacccaaaaggatattgatgaagctccacaagttcctcatgttccaccattattgccagaagataagagataa

SEQ ID NO: 5 - DNA, 1104 bp, Artificial
  Synthetic gene
atgccatccaagttggccattacctctatgtctttgggtagatgttatgccggtcattctttcactactaagttggatatggctagaaagtacggttaccaaggtttggaattattccatgaagatttggctgatgtcgcctatagattgtctggtgaaactccatctccatgtggtccatcaccagctgctcaattgtctgctgctagacaaattttgagaatgtgccaagtcagaaacatcgaaatcgtttgcttgcaaccattctctcaatacgatggtttgttggatagagaagaacacgaaagaagattggaacaattggaattctggatcgaattggcccatgaattggataccgatattattcaaattccagccaacttcttgccagccgaagaagttactgaagatatctctttgattgtctccgacttgcaagaagtagctgatatgggtttacaagctaacccaccaattagattcgtttacgaagctttgtgttggtccactagagttgatacttgggaaagatcttgggaagttgttcaaagagttaacagaccaaacttcggtgtttgtttggacacttttaacattgccggtagagtttatgctgatccaactgttgcttctggtagaactccaaatgctgaagaagctatcagaaagtccattgccagattggttgaaagagttgacgtttccaaggttttctacgttcaagttgttgatgccgaaaagttgaagaaaccattggttccaggtcacagattctatgatccagaacaaccagctagaatgtcttggtctagaaactgcagattattctacggtgaaaaggatagaggtgcttacttgccagtaaaagaaattgcttgggcttttttcaacggtttgggttttgaaggttgggtttccttagaattattcaacagaagaatgtccgataccggttttggtgttccagaagaattagctagaagaggtgctgtttcttgggctaaattggttagagatatgaagatcaccgttgactctccaactcaacaacaagctacacaacaacctatcagaatgttgtctttgtcagctgctttgtga

SEQ ID NO: 6 - DNA, 915 bp, Artificial
  Synthetic gene
atggaattgagacacttgagatacttcgttgccgttgttgaagaacaatcttttacaaaggctgccgacaagttgtgtattgctcaaccaccattatccagacaaatccaaaacttggaagaagaattgggtatccaattattggaaagaggttccagaccagttaagactactccagaaggtcatttcttttaccaatacgccatcaagttgttgtccaacgttgatcaaatggtcagtatgaccaagagaattgcctctgttgaaaagaccattagaatcggttttgttggttccttgttgttcggtttgttgccaagaattatccacttgtacagacaagctcatccaaacttgagaatcgaattatacgaaatgggtactaaggctcaaaccgaagctttgaaagaaggtagaattgacgctggttttggtagattgaagatttctgatccagccatcaagagaaccttgttgagaaacgaaagattgatggttgctgttcatgcttcccatccattgaatcaaatgaaggataagggtgttcacttgaacgatttgatcgacgaaaagatcttgttgtacccatcttctccaaagccaaacttctctactcatgttatgaacatcttctctgaccatggtttggaacctaccaagattaacgaagttagagaagtccaattggccttgggtttggttgctgctggtgaaggtatttcattggttccagcttctacccaatccattcaattattcaacttgtcctacgtcccattattagatccagatgctattaccccaatctacattgctgttagaaacatggaagaatccacctacatctactcattatacgaaaccatcagacaaatctacgcctacgaaggttttactgaaccaccaaattggtaa

SEQ ID NO: 7 - DNA, 930 bp, Artificial
  Synthetic gene
atgcgtttcaacaagctcgacctcaatcttctggtcgccctggatgcactgctcacggagatgagcatcagccgcgccgccgaaaagatccatctgagccagtcggccatgagcaatgccctggcgcggctgcgcgagtatttcgatgatgaattgctgatccaggtgggccggcgcatggagcccacgccgcgcgccgaggtgctcaaggatgcggtgcatgatgtgctgcggcgtatcgatggctccatcgcggcgctgccggccttcgtgccggccgagtccacgcgcgagtttcgcatctcggtttcggactttacgctctccgtcctcatcccccgggtgctggcgcgcgcgcacgccgagggcaagcacatccgctttgccctgatgccgcaggtgcaagacccgacccgctcgctggatcgggccgaggtggacctgctggtcttgccgcaggaattctgcacgcccgatcatcctgccgaagaggtcttccgcgaacggcatgtctgcgtggtctggcgcgacagtgcgctggcgcaaggcgagctgacgctggaacgctacatggcctcaggccatgtggtgatggtgccgcctggggccaatgcgtcgtcggtggaggcgtggatggccaggaagctgggctttgcgcgccgggtggaagtgaccagcttcagcttcgcttctgcgctggcgctggtacaggggacggaccgcatcgccacggtgcatgcccggctggcgcagctgctggctccgcaatggccggtggtgatcaaggagagtccgctgtcgctgggcgagatgcggcagatgatgcagtggcatcgctaccgcagcaatgatcctggcatccagtggctgcgtcgggtgtttctggagagtgcgcaggagatggatgcggcgctgccaggcatctgctga

SEQ ID NO: 8 - DNA, 942 bp, Artificial
  Synthetic gene
atgattgatgcacgtgtgaaatttagacatttgcaaacttttgtagaagttgctagacaaaagagtgttgtaaaagcagccgaattattacatgtaacacagccagcagtgactaagaccataagggaattggaagaggtattaggtgtcgccgtgtttgaaagagaaggtcgtggtatcaaaataacaagatatggggaagtttttttgagacatgcaggagctgcccttacggctcttcgtcaaggtctagacagcgtatctcaagaaagaagtggcgaaggtccaccaatcagggtaggcgccttacctacagtatcaactagaatcatgccaagagctattgcactttttctgaaggaaaaaacgggtgcaagaattaaaatagtcacaggcgaaaatgcggtattgcttgaacaattgagaatcggcgacctagacttggttgtgggaaggcttgccgccccggataaaatgactgggttttctttcgagcacctatacagtgagcaagttgtgtttgcagtaagggcaggccatcccctgatctccggtaggcaatccttgtttgctcatctttccgactaccctgttctaatgccaacaagggccagcataattaggccattcgtcgagcactttttgatagctaatggcatcgctggtttgccaaaccagatagaaaccgtctccgattcatttggtagagcttttgtacgttcttccgacgctatttggattatatccgctggtgtagtagctactgatattgccgatggtgttttggcagctctaccagtagacacttcagaaacccgtggccctgttggcttgactatgagaaccgatgcaataccatctttgcctctttcaatcttaatgcaaactttaagagaagtggccggtaccgcaatggcagctgaagccaaaagaacagcataa

SEQ ID NO: 9 - DNA, 894 bp, Artificial
  Synthetic gene
atgaaacgtcctgattatagaactctgcaagccttagatgctgtaattagagaacgtggcttcgagagagcggctcagaagttgtgtattactcaatccgccgtgagccagagaataaagcagctagaaaatatgtttggccaaccattactggtacgtactgttcctcctaggccgacggaacaaggtcagaagcttttggccttgttgagacaagtggagttgctagaagaggaatggttgggagacgagcagacaggttcaacaccacttttattgagtctggccgtaaatgcggatagcctagctacttggttgctaccggctctagctcctgtcttggctgacagtcccataagattaaacttacaagtcgaagatgaaacgagaacgcaagaaagacttaggagaggagaggtcgtgggggctgtatcaattcaacatcaggcattgcccagttgtttagtcgacaagttgggtgcgctagattaccttttcgtgtcatccaaacctttcgccgagaagtattttcctaatggcgttacccgttccgctttgcttaaagccccagtcgtagcattcgaccatctagatgacatgcaccaagcctttttacaacaaaatttcgatttaccaccaggctccgttccatgccatatcgtgaactcttccgaagccttcgtacaactagctcgtcaaggtactacttgctgtatgattccacatctacaaatagaaaaagaattggcctccggagaattgatagacctgacacctggcctatttcaaaggagaatgctgtattggcataggtttgcaccagagtcaagaatgatgagaaaggtgactgatgcattgcttgattatggccataaggtgttaagacaagattga

SEQ ID NO: 10 - DNA, 927 bp, Artificial
  Synthetic gene
atgaaggacgacatcaatcaagaaattaccttcaggaagttatctgttttcatgatgtttatggccaaaggcaatatcgccagaactgctgaagcaatgaagttatcatctgtgtcagttcacagagcgctgcatacactagaagaaggtgtgggatgtcccctgttcgtccacaaaggtagaaatctactacctctacaggcagcatggactctattagaatattgccaagatgtaatttcattaatgaatagaggactagaagccactagaaaagtggcaggtgttggtcaaggaagattgagaatcggtacactttactccttaacactagaaaccgtaccaaggataataatgggcatgaagttaagacgtccagaacttgagctagacttgacaatgggttcaaatcaaatgttattagatatgctagaagatgatgccttagatgcaatattgatagctaccaacgaaggcgaattcaacaatactgcctttgatgttgttcctttgtttgaggatgacatatttcttgcagcacctgcaactgaacgtcttgacgcctcaagattggctgacctgagagattacgctgatagaaagtttgtttccttagcggaaggatttgctacctatgctggttttcgtgaagctttccatatagctggctttgaaccagagatagttaccagagttaatgacatattcagtatgatatctcttgttcaggctggtgttgggtttgctcttttgccaggaagaatgaagaaagtttatgaaaaggacgttcaattgcttaagttagccgaaccttaccaaatgagacagctgattagtatcgtatattcccatcacagggaacgtgacgctgatttgttggcattagcggctgaaggtaggatgtatgctcgttctattaacaggtaa

SEQ ID NO: 11 - DNA, 927 bp, Artificial; identical to SEQ ID NO: 10
  Synthetic gene

SEQ ID NO: 12 - DNA, 1014 bp, Artificial
  Synthetic gene
ccaggcaactttagtgctgacacataagcttgatattgatcaaatggattgttttgattcatgatatggacggcatcaatacattgaccaccccatccgcaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaataatctgtcataaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataa

SEQ ID NO: 13 - DNA, 1020 bp, Artificial
  Synthetic gene
ccaggcaactttagtgctgacacatagatcgtataacctcctggttaagggaaagccacgaaatatcattttacctaaccggatgaaacatccaaatctgacgacgcaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaataatctgtcataaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaata

SEQ ID NO: 14 - DNA, 1028 bp, Artificial
  Synthetic gene
ccaggcaactttagtgctgacacatatctggcctctctcttattagtttttctgattgccaattaatattatcaatttccgctaataacaatcccgcgatatagtctctgcatcaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaataatctgtcataaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataa

SEQ ID NO: 15 - DNA, 1030 bp, Artificial
  Synthetic gene
ccaggcaactttagtgctgacacataatcgttactctgatgctaacgatcggccaccgcgcttaattgatgctcatagcctcgcgtcgcacactaatctccaccaggacaaacaacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaataatctgtcataaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataa

SEQ ID NO: 16 - DNA, 1317 bp, Artificial
  Synthetic gene
atgggtgaactcaaggaaatcttgaaacagaggtatcatgagttgcttgactggaatgtcaaagcccctcatgtccctctcagtcaacgactgaagcattttacatggtcttggtttgcatgtactatggcaactggtggtgttggtttgattattggttctttcccctttcgattttatggtcttaatacaattggcaaaattgtttatattcttcaaatctttttgttttctctctttggatcatgcatgctttttcgctttattaaatatccttcaactatcaaggattcctggaaccatcatttggaaaagcttttcattgctacttgtcttctttcaatatccacgttcatcgacatgcttgccatatacgcctatcctgataccggcgagtggatggtgtgggtcattcgaatcctttattacatttacgttgcagtatcctttatatactgcgtaatggctttttttacaattttcaacaaccatgtatataccattgaaaccgcatctcctgcttggattcttcctattttccctcctatgatttgtggtgtcattgctggcgccgtcaattctacacaacccgctcatcaattaaaaaatatggttatctttggtatcctctttcaaggacttggtttttgggtttatcttttactgtttgccgtcaatgtcttacggttttttactgtaggcctggcaaaaccccaagatcgacctggtatgtttatgtttgtcggtccaccagctttctcaggtttggccttaattaatattgcgcgtggtgctatgggcagtcgcccttatatttttgttggcgccaactcatccgagtatcttggttttgtttctacctttatggctatttttatttggggtcttgctgcttggtgttactgtctcgccatggttagctttttagcgggctttttcactcgagcccctctcaagtttgcttgtggatggtttgcattcattttccccaacgtgggttttgttaattgtaccattgagataggtaaaatgatagattccaaagctttccaaatgtttggacatatcattggggtcattctttgtattcagtggatcctcctaatgtatttaatggtccgtgcgtttctcgtcaatgatctttgctatcctggcaaagacgaagatgcccatcctccaccaaaaccaaatacaggtgtccttaaccctaccttcccacctgaaaaagcacctgcatctttggaaaaagtcgatacacatgtcacatctactggtggtgaatcggatcctcctagtagtgaacatgaaagcgtttaa

SEQ ID NO: 17 - DNA, 2154 bp, Artificial
  Synthetic gene
atggaccaaattgaagcaatgctatgcggtggtggtgaaaagaccaaggtggccgtaacgacaaaaactcttgcagatcctttgaattggggtctggcagctgaccagatgaaaggtagccatctggatgaagttaagaagatggttgaggaatacagaagaccagtcgtaaatctaggcggcgagacattgacgataggacaggtagctgctatttcgaccgttggcggttcagtgaaggtagaacttgcagaaacaagtagagccggagttaaggcttcatcagattgggtcatggaaagtatgaacaagggcacagattcctatggcgttaccacaggctttggtgctacctctcatagaagaactaaaaatggcactgctttgcaaacagaactgatcagattccttaacgccggtattttcggtaatacaaaggaaacttgccatacattaccccaatcggcaacaagagctgctatgcttgttagggtgaacactttgttgcaaggttactctggaataaggtttgaaattcttgaggccatcacttcactattgaaccacaacatttctccttcgttgcccttaagaggaacaataactgccagcggtgatttggttcccctttcatatatcgcaggcttattaacgggaagacctaattcaaaggccactggtccagacggagaatccttaaccgctaaggaagcatttgagaaagctggtatttcaactggtttctttgatttgcaacccaaggaaggtttagccctggtgaatggcaccgctgtcggcagcggtatggcatccatggtgttgtttgaagctaacgtacaagcagttttggccgaagttttgtccgcaatttttgccgaagtcatgagtggaaaacctgagtttactgatcacttgacccacaggttaaaacatcacccaggacaaattgaagcagcagctatcatggagcacattttggacggctctagctacatgaagttagcccagaaggttcatgaaatggaccctttgcaaaaacccaaacaagatagatatgctttaaggacatccccacaatggcttggccctcaaattgaagtaattagacaagctacaaagtctatagaaagagagatcaactctgttaacgataatccacttattgatgtgtcgaggaataaggcaatacatggaggcaatttccagggtacacccataggagtcagtatggataataccaggcttgccatagccgcaattggcaaattaatgtttgcccaattttctgaattggtcaatgacttctacaataacggtttgccttcgaatctgaccgcatcttctaaccctagtcttgattatggtttcaaaggtgctgagatagcaatggcaagctattgttcagagctgcaatatctagccaacccagtaacctctcatgtacaatcagccgaacaacacaatcaggatgttaattctttgggcctgatttcatcaagaaaaacaagcgaggccgttgatatccttaaattaatgtccacaacatttttagtgggtatatgccaggccgtagatttgagacacttggaagagaatttgagacagacagtgaaaaataccgtatcacaggttgcaaaaaaggttctaactacaggtatcaatggtgaattgcacccatcaagattctgtgaaaaagatttattaaaagttgtagatagagaacaagtatttacttacgttgacgatccatgtagcgctacttatccattgatgcagagattgagacaagttattgtagatcacgctttatccaatggtgaaactgagaaaaatgccgttacttcaatattccaaaagataggtgcctttgaagaagaactgaaggcagttttaccaaaggaagtcgaagctgctagagccgcatacggaaatggtactgcccctataccaaatagaatcaaagagtgtaggtcgtaccctttgtacagattcgttagagaagagttgggaaccaaattactaactggtgaaaaagtcgttagcccaggtgaagaatttgacaaggtattcacagctatgtgcgagggaaagttgatagatccacttatggattgcttgaaagagtggaatggtgcacctattccaatctgctaa

SEQ ID NO: 18 - DNA, 3702 bp, Artificial
  Synthetic gene
atggatttgttattgctggaaaagtcacttattgctgtatttgtggcagttattctagccacggttatttctaaattaagaggtaagaaactaaaactacctcctggtcccatccccataccaatttttggtaattggttgcaagtgggcgatgatttgaatcacagaaatttggtagactatgctaagaagttcggtgaccttttcttgcttagaatgggtcaaaggaatttggtagtggttagctcacctgatttgactaaggaggtcttattaacgcaaggcgttgagtttggctccagaactagaaatgttgtgtttgatattttcactggtaaaggtcaagatatggtttttacagtttacggtgagcactggagaaaaatgagaagaatcatgaccgtaccattctttactaacaaggttgttcaacaaaatagagaaggttgggagtttgaggcagcttccgtagtggaagacgtaaagaaaaatccagattcggccacaaagggtatagtactaagaaaaagactacaattgatgatgtacaacaatatgttcagaattatgtttgacagaagatttgaaagtgaagatgaccctttgttcctgagacttaaggctttgaatggtgaaagatcgagattggctcaaagtttcgaatataattacggtgactttattccaatcttaagaccatttttgagaggctatttgaaaatttgccaagacgtcaaggataggaggatcgctcttttcaagaagtactttgtggacgagagaaagcaaatagcttcttccaagcccacaggttcggaaggtttaaaatgtgcaattgatcatattttagaagctgaacaaaaaggtgaaattaacgaagataatgttttgtacattgtagaaaatatcaatgtggctgcaatagaaacaaccttatggtcaatagaatggggtattgctgaattggtgaatcacccagaaatacaatctaaactgagaaacgagctagataccgttttaggtccaggtgtccaagttacagaacctgatttgcataagttaccctacttgcaagctgtggttaaagaaaccttgagattgagaatggctattcctcttctagttcctcatatgaacctacatgatgctaaactggccggttatgatattccagcagaaagtaagattttagtaaatgcatggtggttggccaacaatccaaacagttggaaaaagcctgaagaattcagacctgaaagattcttcgaagaggaatctcatgttgaagccaacggaaatgacttcagatatgtaccttttggcgttggcagaagatcgtgtccaggaataatactagccttaccaatattgggtatcacaattggtaggatggttcaaaattttgagttgctaccaccacccggacaatcgaaagtcgatacttcagagaaaggaggacaattctcattgcatattttgaatcattccattatagtcatgaaacccagaaattgtagcgctgaagctgcagcaaaagaagctgcagctaaagaagctgcagcaaaagcttccagtagctcttcctcctcaacctcgatgatcgacttaatggctgctattataaaaggagaaccagttatagttagtgaccctgctaacgcaagcgcttacgaatccgttgcagccgagttgtcaagtatgcttatagaaaatagacagtttgctatgattgtaacgaccagcatcgccgttttaattggttgcatcgtgatgttggtgtggaggaggagcggttcgggcaattcaaagagggttgaaccactaaagccattagttatcaaacctagagaagaggaaattgacgatggaaggaagaaagtcactatattcttcggcacccaaacaggtacagctgaaggttttgctaaggctctaggagaagaagcaaaagctagatatgaaaagacgagattcaaaattgtcgatctggatgactatgccgccgatgatgacgaatacgaagaaaaattgaagaaagaagatgtcgcatttttcttccttgccacctacggcgacggtgaaccaacagataatgccgcaaggttttacaagtggtttactgaaggtaatgacagaggagaatggctgaagaatttgaaatatggtgtgttcggccttggtaacagacagtacgagcattttaataaggtcgctaaggttgtagatgatatacttgttgaacaaggtgctcaaaggttagtgcaggtgggcttgggtgacgatgatcaatgtattgaagatgactttactgcttggagagaagccttgtggcctgaattagatactatccttagagaagaaggtgacactgctgttgctaccccctacactgcagcagtcctagaatatagagtctcaatccatgattcagaagacgccaaattcaatgatattaacatggccaacggtaacggttacaccgtttttgacgcacaacatccatacaaagctaatgttgctgttaaaagggaacttcacaccccagaaagtgacaggtcatgtatacatttggaatttgatatcgctggtagtggtttgacttacgaaacaggtgaccatgtcggagtactttgcgataatttgtcagaaactgttgatgaagctttgaggttattggatatgtcaccagatacttacttctcattgcatgcagaaaaagaagacggaactccaatatcaagctcgcttccccctccattccctccctgtaacttaagaacagccctaactagatatgcttgtttactgtcttctccaaagaaaagtgctttggttgcattggcagcccacgcatccgatcctaccgaagctgagagattaaagcatttggcttcaccagccggtaaagatgaatacagtaagtgggtagtggagagccaaagatcgcttttagaagtgatggctgagtttccaagtgctaaacctcctctgggtgtatttttcgctggtgtggccccaagattgcagcctagattttattccatatcctcatctccaaaaattgccgaaaccagaattcacgtgacatgtgctctggtctacgaaaagatgccaacaggtaggattcacaagggtgtctgttctacctggatgaaaaatgctgtaccctatgaaaaatccgaaaattgttctagtgcaccaattttcgtaagacaatctaatttcaagttaccaagcgattctaaagtacccattattatgatcggtccaggtactggtttggccccattcagaggcttcttgcaagaaagattggctttagtggagagtggagttgaattgggtccttcagttttattctttggttgtagaaacagaagaatggactttatctacgaagaagaattgcagagatttgttgaaagtggtgcattggccgaattgagtgttgcattcagcagggaaggtccaaccaaagaatacgttcaacacaagatgatggacaaggcttctgatatctggaatatgatttcccaaggtgcttatttgtatgtttgtggtgacgctaaaggaatggctagagatgttcatagatcactgcatacaatcgcacaagaacaaggtagcatggattcaacaaaagcagagggctttgtaaagaatcttcagacaagcggtagatatctgagagatgtatggtaa

SEQ ID NO: 19 - DNA, 1671 bp, Artificial
  Synthetic gene
atgacgacacaagatgtgatagtcaatgatcagaatgatcagaaacagtgtagtaatgacgtcattttccgatcgagattgcctgatatatacatccctaaccacctcccactccacgactacatcttcgaaaatatctcagagttcgccgctaagccatgcttgatcaacggtcccaccggcgaagtatacacctacgccgatgtccacgtaacatctcggaaactcgccgccggtcttcataacctcggcgtgaagcaacacgacgttgtaatgatcctcctcccgaactctcctgaagtagtcctcactttccttgccgcctccttcatcggcgcaatcaccacctccgcgaacccgttcttcactccggcggagatttctaaacaagccaaagcctccgctgcgaaactcatcgtcactcaatcccgttacgtcgataaaatcaagaacctccaaaacgacggcgttttgatcgtcaccaccgactccgacgccatccccgaaaactgcctccgtttctccgagttaactcagtccgaagaaccacgagtggactcaataccggagaagatttcgccagaagacgtcgtggcgcttcctttctcatccggcacgacgggtctccccaaaggagtgatgctaacacacaaaggtctagtcacgagcgtggcgcagcaagtcgacggcgagaatccgaatctttacttcaacagagacgacgtgatcctctgtgtcttgcctatgttccatatatacgctctcaactccatcatgctctgtagtctcagagttggtgccacgatcttgataatgcctaagttcgaaatcactctcttgttagagcagatacaaaggtgtaaagtcacggtggctatggtcgtgccaccgatcgttttagctatcgcgaagtcgccggagacggagaagtatgatctgagctcggttaggatggttaagtctggagcagctcctcttggtaaggagcttgaagatgctattagtgctaagtttcctaacgccaagctaggtcagggctatgggatgacagaagcaggtccggtgctagcaatgtcgttagggtttgctaaagagccgtttccagtgaagtcaggagcatgtggtacggtggtgaggaacgccgagatgaagatacttgatccagacacaggagattctttgcctaggaacaaacccggcgaaatatgcatccgtggcaaccaaatcatgaaaggctatctcaatgaccccttggccacggcatcgacgatcgataaagatggttggcttcacactggagacgtcggatttatcgatgatgacgacgagcttttcattgtggatagattgaaagaactcatcaagtacaaaggatttcaagtggctccagctgagctagagtctctcctcataggtcatccagaaatcaatgatgttgctgtcgtcgccatgaaggaagaagatgctggtgaggttcctgttgcgtttgtggtgagatcgaaagattcaaatatatccgaagatgaaatcaagcaattcgtgtcaaaacaggttgtgttttataagagaatcaacaaagtgttcttcactgactctattcctaaagctccatcagggaagatattgaggaaggatctaagagcaagactagcaaatggattaatgaactag

SEQ ID NO: 20 - DNA, 1173 bp, Artificial
  Synthetic gene
atggttactgttgaagaagttagaaaagctcaaagggcagaaggtccagccacagtgatggctattggaaccgcagttcctccaaattgtgtagatcaggccacttatcctgactactactttagaataacaaactctgagcataaggctgaattgaaagaaaagttccaaaggatgtgcgacaaatcacagatcaagaaaagatacatgtaccttaatgaggaagtcctaaaggaaaacccaaatatgtgtgcatacatggccccttcccttgacgctagacaagatattgtggttgtagaggtcccaaaattgggcaaggaagcagctgttaaagccataaaggaatggggtcaacctaagagcaaaatcacccaccttgtgttttgcactacaagcggagttgacatgccaggcgcagattatcagctaaccaaacttttgggtttaaggccttctgtaaaaagattgatgatgtaccaacaaggttgtttcgctggaggcactgtcttaagactagccaaggatcttgcagagaacaacaaaggtgctagggtgttggttgtatgctcagaaattacagccgtcacctttagaggaccaactgacactcacttagattccctagttggtcaggcattgtttggcgacggtgctgccgcaataatcattggaagtgatcctattccagaggtggaaaagcctctttttgaacttgttagcgctgcccaaactatattgccagattctgagggtgcaatcgacggccacttaagggaagtaggtctaaccttccatcttttgaaagatgtccctggtttaatttcaaagaacgtggaaaaatccctaacagaggcttttaaaccattgggtataagtgactggaatagcttattctggatcgctcacccaggcggccctgccatacttgaccaggttgaagcaaaattgagcttaaagccagaaaaactaagagctactagacatgtattgtcagagtatggtaacatgtccagtgcctgtgtccttttcattttggatgaaatgaggagaaaaagcaaggaggacggcctaaaaaccacaggtgagggaatcgaatggggtgttctattcggctttggtccaggccttactgtggagacagttgtacttcattcagtcgcaattaattag

SEQ ID NO: 21 - DNA, 726 bp, Artificial
  Synthetic gene
atgtctccaccagtttctgttacaaaaatgcaagtcgaaaattatgcttttgcaccaacagtgaaccctgccggttccaccaatactttgttcttagctggagcaggccatagaggtctagagattgaaggaaagtttgtgaaattcacagccataggcgtataccttgaggaaagtgctatcccatttttggcagaaaagtggaaaggtaagacccctcaggagttaactgatagcgtcgagttctttagggacgtggttacaggtccattcgaaaagtttaccagagtaactatgattctacctcttacaggaaagcaatattctgagaaagtcgccgaaaactgtgttgctcactggaagggcataggtacctacactgatgacgaaggaagggcaatcgagaaattcttggatgtgtttagatcagaaacattcccacctggtgcttccattatgtttactcagagtccattaggcttgttaaccatcagctttgccaaggacgattcagttaccggtactgcaaatgctgtaatcgagaacaaacaactatcagaagccgtccttgaatccattattggaaagcatggtgtgagtcctgcagccaaatgctctgttgccgagagagtagcagaattgttaaaaaagagctatgctgaagaggcctcagtgttcggcaaaccagaaaccgaaaagtccacaatacctgttatcggtgtgtag

SEQ ID NO: 22 - DNA, 25 bp, Artificial
  Primer sequence
  misc_feature: n is a deoxyuracil residue
  misc_feature (8)..(8): n is a deoxyuracil residue
cgtgcgangcacacaccatagcttc

SEQ ID NO: 23 - DNA, 26 bp, Artificial
  Primer sequence
  misc_feature (9)..(9): n is a deoxyuracil residue
atgacaganttgtaattaaaacttag

SEQ ID NO: 24 - DNA, 27 bp, Artificial
  Primer sequence
  misc_feature (10)..(10): n is a deoxyuracil residue
agctactgangcacacaccatagcttc

SEQ ID NO: 25 - DNA, 31 bp, Artificial
  Primer sequence
  misc_feature (10)..(10): n is a deoxyuracil residue
atcagtagcnataaaaaacacgctttttcag

SEQ ID NO: 26 - DNA, 34 bp, Artificial
  Primer sequence
  misc_feature (9)..(9): n is a deoxyuracil residue
acctgcacntttgtttgtttatgtgtgtttattc

SEQ ID NO: 27 - DNA, 25 bp, Artificial
  Primer sequence
  misc_feature (9)..(9): n is a deoxyuracil residue
atctgtcanaaaacaatgtcccaag

SEQ ID NO: 28 - DNA, 26 bp, Artificial
  Primer sequence
  misc_feature (8)..(8): n is a deoxyuracil residue
cacgcganttacaacttgatttcagc

SEQ ID NO: 29 - DNA, 26 bp, Artificial
  Primer sequence
  misc_feature (9)..(9): n is a deoxyuracil residue
atctgtcanaaaacaatgatctgtcc

SEQ ID NO: 30 - DNA, 26 bp, Artificial
  Primer sequence
  misc_feature (8)..(8): n is a deoxyuracil residue
cgtgcgantcattcaatttcttgagc

SEQ ID NO: 31 - DNA, 30 bp, Artificial
  Primer sequence
  misc_feature (9)..(9): n is a deoxyuracil residue
atctgtcanaaaacaatgaccgccccaatc

SEQ ID NO: 32 - DNA, 30 bp, Artificial
  Primer sequence
  misc_feature (8)..(8): n is a deoxyuracil residue
cacgcganttacttagcggaaccttgattc

SEQ ID NO: 33 - DNA, 26 bp, Artificial
  Primer sequence
  misc_feature (9)..(9): n is a deoxyuracil residue
atctgtcanaaaacaatgatctgtcc

SEQ ID NO: 34 - DNA, 26 bp, Artificial
  Primer sequence
  misc_feature (8)..(8): n is a deoxyuracil residue
cacgcganttatctcttatcttctgg

SEQ ID NO: 35 - DNA, 27 bp, Artificial
  Primer sequence
  misc_feature (9)..(9): n is a deoxyuracil residue
agtgcaggnaaaacaatgccatccaag

SEQ ID NO: 36 - DNA, 28 bp, Artificial
  Primer sequence
  misc_feature (8)..(8): n is a deoxyuracil residue
cgtgcgantcacaaagcagctgacaaag

SEQ ID NO: 37 - DNA, 37 bp, Artificial
  Primer sequence
  misc_feature (9)..(9): n is a deoxyuracil residue
agtgcaggnaaaacaatgactcaattcactgacattg

SEQ ID NO: 38 - DNA, 31 bp, Artificial
  Primer sequence
  misc_feature (8)..(8): n is a deoxyuracil residue
cgtgcgantcagaaagcttttttcaaaggag

SEQ ID NO: 39 - DNA, 19 bp, Artificial
  Primer sequence
gatgaatgcggccgcttta

SEQ ID NO: 40 - DNA, 24 bp, Artificial
  Primer sequence
caatacgccatcaagttgctaagc

SEQ ID NO: 41 - DNA, 42 bp, Artificial
  Primer sequence
ctccttccttttcggttagagcggatgaatgcggccgcttta

SEQ ID NO: 42 - DNA, 37 bp, Artificial
  Primer sequence
tcatttcttttaccaatacgccatcaagttgctaagc

SEQ ID NO: 43 - DNA, 30 bp, Artificial
  Primer sequence
  misc_feature (9)..(9): n is a deoxyuracil residue
atctgtcanaaaacaatggaattgagacac

SEQ ID NO: 44 - DNA, 28 bp, Artificial
  Primer sequence
  misc_feature (8)..(8): n is a deoxyuracil residue
cacgcganttaccaatttggtggttcag

SEQ ID NO: 45 - DNA, 26 bp, Artificial
  Primer sequence
  misc_feature (8)..(8): n is a deoxyuracil residue
cgtgcganatactccataggtatttt

SEQ ID NO: 46 - DNA, 28 bp, Artificial
  Primer sequence
  misc_feature (8)..(8): n is a deoxyuracil residue
cacgcganttatttgtacaattcatcca

SEQ ID NO: 47 - DNA, 28 bp, Artificial
  Primer sequence
  misc_feature (9)..(9): n is a deoxyuracil residue
atctgtcanaaaacaatgtctaaaggtg

SEQ ID NO: 48 - DNA, 39 bp, Artificial
  Primer sequence
  misc_feature (8)..(8): n is a deoxyuracil residue
cgtgcganttcttaggcacaacaatatttataaaagaag

SEQ ID NO: 49 - DNA, 31 bp, Artificial
  Primer sequence
  misc_feature (9)..(9): n is a deoxyuracil residue
atgacagancgctggatatgcctagaaatgc

SEQ ID NO: 50 - DNA, 36 bp, Artificial
  Primer sequence
  misc_feature (8)..(8): n is a deoxyuracil residue
cgtgcganggaaaaccaagaaatgaattatatttcc

SEQ ID NO: 51 - DNA, 40 bp, Artificial
  Primer sequence
  misc_feature (9)..(9): n is a deoxyuracil residue
atgacagantattaatttagtgtgtgtatttgtgtttgtg

SEQ ID NO: 52 - DNA, 31 bp, Artificial
  Primer sequence
  misc_feature (8)..(8): n is a deoxyuracil residue
cgtgcganccaggcaactttagtgctgacac

SEQ ID NO: 53 - DNA, 33 bp, Artificial
  Primer sequence
  misc_feature (8)..(8): n is a deoxyuracil residue
cgtgcganataaaaaacacgctttttcagttcg

SEQ ID NO: 54 - DNA, 36 bp, Artificial
  Primer sequence
  misc_feature (9)..(9): n is a deoxyuracil residue
atgacagantttgtttgtttatgtgtgtttattcga

SEQ ID NO: 55 - DNA, 27 bp, Artificial
  Primer sequence
  misc_feature (8)..(8): n is a deoxyuracil residue
cgtgcgangaaagaccacacccacgcg

SEQ ID NO: 56 - DNA, 40 bp, Artificial
  Primer sequence
  misc_feature (9)..(9): n is a deoxyuracil residue
atgacaganggtaattggacaaataaatacgtgtattaag

SEQ ID NO: 57 - DNA, 26 bp, Artificial
  Primer sequence
  misc_feature (8)..(8): n is a deoxyuracil residue
cgtgcgantcaagcccacgcgtaggc

SEQ ID NO: 58 - DNA, 26 bp, Artificial
  Primer sequence
  misc_feature (8)..(8): n is a deoxyuracil residue
cgtgcgantcgagcagatccgccagg

SEQ ID NO: 59 - DNA, 27 bp, Artificial
  Primer sequence
  misc_feature (8)..(8): n is a deoxyuracil residue
cacgcgantcagcagatgcctggcagc

SEQ ID NO: 60 - DNA, 36 bp, Artificial
  Primer sequence
  misc_feature (9)..(9): n is a deoxyuracil residue
atctgtcanaaaacaatgcgtttcaacaagctcgac

SEQ ID NO: 61 - DNA, 30 bp, Artificial
  Primer sequence
  misc_feature (9)..(9): n is a deoxyuracil residue
atctgtcanaaaacaatgattgatgcacgt

SEQ ID NO: 62 - DNA, 29 bp, Artificial
  Primer sequence
  misc_feature (8)..(8): n is a deoxyuracil residue
cacgcganttatgctgttcttttggcttc

SEQ ID NO: 63 - DNA, 29 bp, Artificial
  Primer sequence
  misc_feature (9)..(9): n is a deoxyuracil residue
atctgtcanaaaacaatgaaacgtcctga

SEQ ID NO: 64 - DNA, 32 bp, Artificial
  Primer sequence
  misc_feature (8)..(8): n is a deoxyuracil residue
cacgcgantcaatcttgtcttaacaccttatg

SEQ ID NO: 65 - DNA, 29 bp, Artificial
  Primer sequence
  misc_feature (9)..(9): n is a deoxyuracil residue
atctgtcanaaaacaatgaaggacgacat

SEQ ID NO: 66 - DNA, 32 bp, Artificial
  Primer sequence
  misc_feature (8)..(8): n is a deoxyuracil residue
cacgcganttacctgttaatagaacgagcata

SEQ ID NO: 67 - DNA, 19 bp, Artificial
  Primer sequence
cgaagaaggcctgcaattc

SEQ ID NO: 68 - DNA, 19 bp, Artificial
  Primer sequence
ggccctgataaggttgttg

SEQ ID NO: 69 - DNA, 20 bp, Artificial
  Primer sequence
gaactgacgtcgaaggctct

SEQ ID NO: 70 - DNA, 21 bp, Artificial
  Primer sequence
cgtgaaatctctttgcggtag

SEQ ID NO: 71 - DNA, 20 bp, Artificial
  Primer sequence
tgacgaatcgttaggcacag

SEQ ID NO: 72 - DNA, 19 bp, Artificial
  Primer sequence
ccgtgcaataccaaaatcg

SEQ ID NO: 73 - DNA, 20 bp, Artificial
  Primer sequence
ctcacaaagggacgaatcct

SEQ ID NO: 74 - DNA, 19 bp, Artificial
  Primer sequence
gacggtacgttgaccagag

SEQ ID NO: 75 - DNA, 23 bp, Artificial
  Primer sequence
cttaatgggtagtgcttgacacg

SEQ ID NO: 76 - DNA, 21 bp, Artificial
  Primer sequence
gaagacccatggttccaagga

SEQ ID NO: 77 - DNA, 20 bp, Artificial
  Primer sequence
gtgcttgatttgcgtcattc

SEQ ID NO: 78 - DNA, 20 bp, Artificial
  Primer sequence
cctgcaggactagtgctgag

SEQ ID NO: 79 - DNA, 27 bp, Artificial
  Primer sequence
gttgacacttctaaataagcgaatttc

SEQ ID NO: 80 - DNA, 28 bp, Artificial
  Primer sequence
aaaaataaatagggacctagacttcagg

SEQ ID NO: 81 - DNA, 22 bp, Artificial
  Primer sequence
ctgcaggaattcgatatcaagc

SEQ ID NO: 82 - DNA, 26 bp, Artificial
  Primer sequence
tcaattgagatgagcttaatcatgtc

SEQ ID NO: 83 - DNA, 27 bp, Artificial
  Primer sequence
attattacagtcactcagacagagcac

SEQ ID NO: 84 - DNA, 22 bp, Artificial
  Primer sequence
gtgaagtgatcatgcacatcgc

SEQ ID NO: 85 - DNA, 27 bp, Artificial
  Primer sequence
gaggttccagaccagttaagactactc

SEQ ID NO: 86 - DNA, 22 bp, Artificial
  Primer sequence
gtaagccagattaaaattcacg

SEQ ID NO: 87 - DNA, 22 bp, Artificial
  Primer sequence
tagcatcaccttcaccttcacc

SEQ ID NO: 88 - DNA, 24 bp, Artificial
  Primer sequence
cctgaaattattcccctacttgac

SEQ ID NO: 89 - DNA, 21 bp, Artificial
  Primer sequence
tatcggataacaacaccgctg

SEQ ID NO: 90 - DNA, 20 bp, Artificial
  Primer sequence
ctgttcacccagacacctac

SEQ ID NO: 91 - DNA, 20 bp, Artificial
  Primer sequence
gcggagtccgagaaaatctg

SEQ ID NO: 92 - DNA, 27 bp, Artificial
  Primer sequence
tctcaggtatagcatgaggtcgctcat

SEQ ID NO: 93 - DNA, 28 bp, Artificial
  Primer sequence
cctgcaggactagtgctgaggcattaat

SEQ ID NO: 94 - DNA, 20 bp, Artificial
  Primer sequence
gtttgtagttggcggtggag

SEQ ID NO: 95 - DNA, 20 bp, Artificial
  Primer sequence
gagacaagatggggcaagac

SEQ ID NO: 96 - DNA, 20 bp, Artificial
  Primer sequence
ggcttgtggtcacctgtcat

SEQ ID NO: 97 - DNA, 23 bp, Artificial
  Primer sequence
gaattatggtaattttgattatc

SEQ ID NO: 98 - DNA, 20 bp, Artificial
  Primer sequence
tgcgacagaagaaagggaag

SEQ ID NO: 99 - DNA, 21 bp, Artificial
  Primer sequence
gagaacgagaggacccaacat

SEQ ID NO: 100 - DNA, 37 bp, Artificial
  Primer sequence
  misc_feature (10)..(10): n is a deoxyuracil residue
agcgatacgnaaaatggatttgttattgctggaaaag

SEQ ID NO: 101 - DNA, 34 bp, Artificial
  Primer sequence
  misc_feature (8)..(8): n is a deoxyuracil residue
cacgcganttaccatacatctctcagatatctac

SEQ ID NO: 102 - DNA, 35 bp, Artificial
  Primer sequence
  misc_feature (10)..(10): n is a deoxyuracil residue
atcaacgggnaaaatggaccaaattgaagcaatgc

SEQ ID NO: 103 - DNA, 31 bp, Artificial
  Primer sequence
  misc_feature (8)..(8): n is a deoxyuracil residue
cgtgcganttagcagattggaataggtgcac

SEQ ID NO: 104 - DNA, 37 bp, Artificial
  Primer sequence
  misc_feature (10)..(10): n is a deoxyuracil residue
agcgatacgnaaaatgacgacacaagatgtgatagtc

SEQ ID NO: 105 - DNA, 32 bp, Artificial
  Primer sequence
  misc_feature (8)..(8): n is a deoxyuracil residue
cacgcganctagttcattaatccatttgctag

SEQ ID NO: 106 - DNA, 36 bp, Artificial
  Primer sequence
  misc_feature (10)..(10): n is a deoxyuracil residue
agcgatacgnaaaatgtctccaccagtttctgttac

SEQ ID NO: 107 - DNA, 31 bp, Artificial
  Primer sequence
  misc_feature (8)..(8): n is a deoxyuracil residue
cacgcganctacacaccgataacaggtattg

SEQ ID NO: 108 - DNA, 31 bp, Artificial
  Primer sequence
  misc_feature (8)..(8): n is a deoxyuracil residue
cgtgcgantaattaattgcgactgaatgaag

SEQ ID NO: 109 - DNA, 36 bp, Artificial
  Primer sequence
  misc_feature (10)..(10): n is a deoxyuracil residue
atcaacgggnaaaatggttactgttgaagaagttag

SEQ ID NO: 110 - DNA, 51 bp, Artificial
  Primer sequence
  misc_feature (10)..(10): n is a deoxyuracil residue
agctgcagcnaaagaagctgcagcaaaagcttccagtagctcttcctcctc

SEQ ID NO: 111 - DNA, 52 bp, Artificial
  Primer sequence
  misc_feature (10)..(10): n is a deoxyuracil residue
agctgcagcntcttttgctgcagcttcagcgctacaatttctgggtttcatg

SEQ ID NO: 112 - DNA, 37 bp, Artificial
  Primer sequence
  misc_feature (10)..(10): n is a deoxyuracil residue
acccgttganttttgtttgtttatgtgtgtttattcg

SEQ ID NO: 113 - DNA, 35 bp, Artificial
  Primer sequence
  misc_feature (8)..(8): n is a deoxyuracil residue
cacgcgangatctcagttcgagtttatcattatca

SEQ ID NO: 114 - DNA, 39 bp, Artificial
  Primer sequence
  misc_feature (10)..(10): n is a deoxyuracil residue
acccgttgangccgcttgttttatatttgttgtaaaaag

SEQ ID NO: 115 - DNA, 33 bp, Artificial
  Primer sequence
  misc_feature (8)..(8): n is a deoxyuracil residue
cacgcganggcctggaagtaccttcaaagaatg

SEQ ID NO: 116 - DNA, 34 bp, Artificial
  Primer sequence
  misc_feature (8)..(8): n is a deoxyuracil residue
cgtgcgangccgcacacaccatagcttcaaaatg

SEQ ID NO: 117 - DNA, 30 bp, Artificial
  Primer sequence
  misc_feature (10)..(10): n is a deoxyuracil residue
acgtatcgcngtgagtcgtattacggatcc

SEQ ID NO: 118 - DNA, 31 bp, Artificial
  Primer sequence
  misc_feature (8)..(8): n is a deoxyuracil residue
cgtgcgangccgatctatgcgactgggtgag

SEQ ID NO: 119 - DNA, 40 bp, Artificial
  Primer sequence
  misc_feature (10)..(10): n is a deoxyuracil residue
acgtatcgcnttttgatagatttgactgtgttattttgcg

SEQ ID NO: 120 - DNA, 29 bp, Artificial
  Primer sequence
  misc_feature (10)..(10): n is a deoxyuracil residue
acccgttganttttgtttgtttatgtgtg

SEQ ID NO: 121 - DNA, 30 bp, Artificial
  Primer sequence
  misc_feature (10)..(10): n is a deoxyuracil residue
acgtatcgcntgtttagttaattatagttc

SEQ ID NO: 122 - DNA, 30 bp, Artificial
  Operator sequence
gccggcgcatgcgccgccggccagccataa

SEQ ID NO: 123 - DNA, 16 bp, Artificial
  Operator sequence
ctatgtcttttgttag

SEQ ID NO: 124 - DNA, 13 bp, Artificial
  Operator sequence
ttcggactccgaa

SEQ ID NO: 125 - DNA, 49 bp, Artificial
  Operator sequence
agattagtaaaattgattgttgggatagctatcatccacgatatggatg

SEQ ID NO: 126 - DNA, 80 bp, Artificial
  Operator sequence
ccgaaaaagtaccgaacatccgtaaatctggataacgttctgcacaatccggatagccccccgccagccgtctccctaac

SEQ ID NO: 127 - DNA, 60 bp, Artificial
  Operator sequence
taaaatcgtaccacttattactaaaaactttttctacacaaaactaagttagtatctaac

SEQ ID NO: 128 - DNA, 51 bp, Artificial
  Operator sequence
tgttgaaattaaaggcctttagaaacttgaattctatggaccgaactaaaa

SEQ ID NO: 129 - DNA, 17 bp, Artificial
  Operator sequence
aacaacctaagtttgca

SEQ ID NO: 130 - DNA, 30 bp, Artificial
  Operator sequence
tttgcatagcgtgaatatgtcaaaattgat

SEQ ID NO: 131 - DNA, 355 bp, Artificial
  Operator sequence
cgatttgccattaatcccattaggactttcgtatcggagaagccttcaacgttattaaacatcattgctggaccttcttgcgtcggccgttttaccgtccctccagcaccaatatagcggtaaacaccagccaattcagcatttggattcacagctacgttcgtctcatggtactggttggcatgggtttttagctcggccaatacttttcgtaaatcataaggatcatttaccatcagattacctcctataagttgcttacaatcaccactttaaggcataaaatcgttgcaaacaactcaactttcgactaatgttatgcctaaatggaataataagaagaaggttcttcaat

SEQ ID NO: 132 - DNA, 250 bp, Artificial
  Operator sequence
tcaggcaggatccgctaatcttatggacaaaaatgctaatgctttgcaaagtgtgacgctgtgcaaatattcaatgtggacattccagccatagttatagacacttctgttacttaattttatcgcctgaactgtacgcttttgttacaaagcgcttttcacaagcggggttgatacgtgctttcatcaagcgcaaagtcttgcggagacggaagctctgtcgtcctggtcgatatggacaatttgtttc

SEQ ID NO: 133 - DNA, 550 bp, Artificial
  Promoter sequence
aatttttttggaaaaccaagaaatgaattatatttccgtgtgagacgacatcgtcgaatatgattcagggtaacagtattgatgtaatcaatttcctacctgaatctaaaattcccgggagcaagatcaagatgttttcaccgatctttccggtctctttggccggggtttacggacgatggcagaagaccaaagcgccagttcatttggcgagcgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaataatgactgaattcaaggccggttctgctaagaaaggtgctacacttttcaa

SEQ ID NO: 134 - DNA, 77 bp, Artificial
  Promoter sequence
atacatactccataggtattttattatacaaataatgtgtttgaacttattaaaacattcttttaaggtataaacaa

SEQ ID NO: 135 - DNA, 73 bp, Artificial
  Promoter sequence
agcttgatattgatcaaatggattgttttgattcatgatatggacggcatcaatacattgaccaccccatccg

SEQ ID NO: 136 - DNA, 80 bp, Artificial
  Promoter sequence
gatcgtataacctcctggttaagggaaagccacgaaatatcattttacctaaccggatgaaacatccaaatctgacgacg

SEQ ID NO: 137 - DNA, 87 bp, Artificial
  Promoter sequence
tctggcctctctcttattagtttttctgattgccaattaatattatcaatttccgctaataacaatcccgcgatatagtctctgcat

SEQ ID NO: 138 - DNA, 670 bp, Artificial Sequence
  Prokaryotic  operator benO for the prokaryotic transcriptional activator BenM placed 6 bp upstream of the TATA box the CMV promoter
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtggatactccataggtattttattatacaaataatgtgtttgaacttattaaaacattcttttaaggtataaacaagaggtctatataagcagagctcgccaccatg

(19) United States
(12) Patent Application Publication
(10) Pub. No.: US 2019/0185865 A1
(43) Pub. Date: Jun. 20, 2019

(54) USE OF PROKARYOTIC TRANSCRIPTIONAL ACTIVATORS AS METABOLITE BIOSENSORS IN EUKARYOTIC CELLS

(71) Applicant: Danmarks Tekniske Universitet, Kgs. Lyngby (DK)

(72) Inventors: Michael Krogh JENSEN, Copenhagen N (DK); Jay D. KEASLING, Berkeley, CA (US); Mette Louise SKJOEDT, Roedovre (DK); Tim SNOEK, Copenhagen (DK)

(73) Assignee: Danmarks Tekniske Universitet, Kgs. Lyngby (DK)

(21) Appl. No.: 16/311,506

(22) PCT Filed: Jul. 6, 2017
(86) PCT No.: PCT/EP2017/066943

§ 371 Date: Dec. 19, 2018

(30) Foreign Application Priority Data
Jul. 6, 2016 (EP) 16178220.6

(51) Int. Cl. C12N 15/81; C12Q 1/02; C12N 15/85

(52) U.S. Cl. CPC C12N 15/81; C12N 2830/002; C12N 15/85; C12Q 1/02

(57) ABSTRACT

The present invention relates to the use of transcriptional activators from prokaryotic organisms for use in eukaryotic cells, such as yeast as sensors of intracellular and extracellular accumulation of a ligand or metabolite specifically activating this transcriptional activator in a eukaryot, such as yeast cell, such as a cell engineered to produce this ligand. The transcriptional activator controls a promoter upstream of one or more gene, which may include e.g. a reporter gene that may be a fluorescence marker, such as luciferase, green fluorescent protein or a gnee encoding antibiotic resistance.

### FIELD OF THE INVENTION

The present invention relates to the use of transcriptional activators from prokaryotic organisms for use in eukaryotic cells, such as yeast as sensors of intracellular and extracellular accumulation of a ligand or metabolite specifically activating this transcriptional activator in a eukaryote, such as yeast cell, such as a cell engineered to produce this ligand. The transcriptional activator controls a promoter upstream of a gene that may include e.g. a reporter gene that may be a fluorescence marker, such as luciferase or green fluorescent protein.

### BACKGROUND OF THE INVENTION

Whole-cell biocatalysts have proven a tractable path towards sustainable production of bulk and fine chemicals. Yet, screening libraries of cellular designs to identify best-performing biocatalysts is most often a low-throughput endeavour. For this reason the development of biosensors enabling real-time monitoring of product formation has gained significant attention.

Bio-based production of chemicals and fuels is an attractive avenue to reduce dependence on petroleum. For bio-based production, biocatalysts must often be genetically modified in order to increase product titers, rates and yields. However, the current efficiency of genome engineering methods and parts prospecting allow for unprecedented genotype diversity that vastly outstrips our ability to screen for best cell performance.

To meet this demand, bioengineers have started to develop genetically encoded devices and systems that enable screening and selection of better-performing biocatalysts in higher throughput. Genetic devices like oscillators, amplifiers and recorders, which have been developed based on fine-tuned relationships between input and output signals are promising tools for programming and controlling gene expression in living cells. These devices sense extra- or intracellular perturbations and actuate cellular decision-making processes akin to logic gates in electrical circuits. For instance, AND logic gates have been built using inducible expression of two split T7 RNA polymerase domains which control reporter gene expression when used in combination with a complimentary set of altered T7-specific promoters. From a diverse set of inputs, other molecular gating components like RNA aptamers, allosteric regulators and ligand-binding transcription factors have been engineered to control outputs for applications such as high-throughput screening, actuation on cellular metabolism, and evolution-based selection of optimal cell performance.

A key component in many of the reported devices is a ligand-inducible transcriptional regulator. Transcriptional regulators are powerful components finding many uses in genetic designs. Owing to their modular structure, transcriptional regulators have proven to be versatile platforms for genetically encoded Boolean logic functions. In particular, gene switches based on ligand-binding transcriptional repressors bind to genomic targets in the absence of their cognate ligand and thereby repress gene expression of the downstream gene(s), whereas binding between ligand and repressor causes the release of the repressor from the DNA and thereby a de-repression. In NOT gates like this, the simple steric hindrance of RNA polymerase progression, like in the case of the tetracyclin-responsive gene switch TetR, have for decades been used for conditional control of gene expression in both prokaryotic and eukaryotic chassis. Most importantly, transcriptional repressors and other artificial transcriptional regulators can be further engineered—including the addition of nuclear localization signals, destabilization domains and transcriptional activation regions—to repurpose conditional repressors into activators. Though conceptually intriguing and practically relevant, the repurposing and engineering of logic gates can suffer from the inherent need for extensive engineering.

Though most ligand-inducible genetic devices adopted for eukaryotes have historically been founded on transcriptional repressors, a hitherto untapped resource for use in genetic designs is ligand-inducible transcriptional activators. Remarkably, bacterial genomes encode a multitude of ligand-inducible activators amenable for integration into synthetic genetic devices. In bacteria, transcriptional activation takes place through (i) a promoter-centric or (ii) an RNA polymerase-centric mechanism. In the former case a transcriptional activator can bind to an operator site in a promoter thereby improving its ability to guide RNA polymerase to initiate transcription, whereas in the latter case activation relies on interactions with the RNA-polymerase itself such as when a housekeeping a factor is replaced by another a factor. Examples of prokaryotic transcriptional activators used for genetic designs in other non-native prokaryotic chassis include arabinose-inducible AraC and quorum sensing LuxR. However, so far no direct transplantation of prokaryotic ligand inducible transcriptional activators has been reported in eukaryotes. As the number of bulk and fine chemicals produced in eukaryote chassis continues to increase, there is an increasing need to be able to regulate these pathways using any and all available means, including the heretofore untapped prokaryotic ligand-inducible transcriptional activators.

### OBJECT OF THE INVENTION

It is an object of embodiments of the invention to provide eukaryotic cells comprising bacterial transcriptional activator systems functioning in a eukaryotic chassis. Accordingly some genes within the eukaryotic cell are under influence of a bacterial transcriptional activator preferably working in a eukaryotic cell on the endogenous promoter of this eukaryotic cell.

It is an object of embodiments of the invention to provide eukaryotic cells that contain sensors, such as easily visible sensors of intracellular and extracellular accumulation of a ligand or metabolites being produced by this cell.

The inventors of the present invention have applied systematic engineering of multiple parameters to search for a general biosensor design based on small-molecule binding transcriptional activators from the prokaryote super-family of LysR-type transcriptional regulators (LTTRs). The present inventors have identified a design supporting LTTR-dependent activation of reporter gene expression in the presence of cognate small-molecule inducers. As proofs-of principle they have applied the biosensors for in vivo screening of cells producing naringenin or cis, cis-muconic acid at different levels, and show that reporter gene output correlates with product accumulation. The transplantation of prokaryotic transcriptional activators into a eukaryotic chassis illustrates the potential of a hitherto untapped resource for engineering biosensors useful for biotechnological applications.

The present inventors have found a direct onboarding of a prokaryotic transcriptional activator as a biosensor for e.g. cis, cis-muconic acid (CCM) in budding yeast Saccharomyces cerevisiae. Based on a multi-parametric engineering strategy the present inventors identified a functional design for the biosensor. Most importantly the design is applicable to a range of other LTTR-based biosensors founded on small-molecule induced transcriptional activators. As proofs-of-principle two of these biosensors were applied for real-time monitoring of bulk and fine chemical product accumulation in yeast cells engineered to produce CCM and naringenin, respectively. This constitutes the first successful direct transfer of prokaryotic transcriptional activators into a eukaryotic chassis to activate gene expression merely by placing the binding site of a transcriptional activator at a defined location in a reporter promoter and without reconfiguring any other motifs and domains.

Systematic engineering and meticulous characterization have for decades pushed forward the sequence-function understanding of genetic parts and interactions thereof. This has allowed the rational engineering of parts and genetic circuits useful for a range of applications within biotechnology. While most of the genetic devices originate from prokaryotes, transplantation into eukaryotes has been reported for a number bioswitches in order to construct orthogonal genetic devices to control a cellular response to a defined input. Specifically, genetic devices enabling the manipulation of transcription through the transplantation of prokaryote transcriptional repressors have inspired researchers, in their quest for tools to screen, select and actuate on cellular responses. In this study we have shown that ligand-inducible transcriptional activators from the largest family of transcriptional regulators found in prokaryotes, can be ported to eukaryotic chassis and used to measure the level of a small molecule inside the cell and activate transcription. The LTTR-based transcriptional activators function as is in yeast without any further engineering nor the co-expression of other molecular components (i.e. 6 factors). In fact, through a systematic engineering approach we provide a framework from which new ligand-binding transcriptional activators from the LTTR family can be designed through the simple swapping of a candidate LTTR operator sequence into the 209 bp_CYC1p truncated endogenous promoter at a defined position (T1)(FIG. 2a-b, Table 4, and Table 5). Also, and most importantly, we provide two successful proofs-of-principle for such biosensors to screen in vivo for the best-performing biocatalysts.

Compared to many of the studies using transcriptional repressors as biosensors in eukaryotes, the biosensor outputs based on ligand-inducible transcriptional activators presented in this study have lower dynamic ranges falling within one order of magnitude. This is in agreement with the observation from using BenM and FdeR as biosensors in E. coli. This can pose a challenge for their applicability in genetic designs where a larger dynamic range is needed. However, we demonstrated in this study how these biosensors could be subjected to biosensor-based FACS for identification of biosensor designs with improved characteristics (ie. dynamic range), which may expand their applicability for metabolic engineering. For sure, we envision this to be exploitable for high-throughput screening of libraries of genetic designs for metabolites for which there exists no high-throughput screening assay or biosensor.

Apart from dynamic range, another key performance measure for biosensors, is their operational range. In our study we demonstrated how biosensors could be used in laboratory strains with limited engineering towards titer improvements, which at their best still are far from commercially relevant. Indeed, in diploid yeast, production of 559.3 mg/L CCM was recently reported, whereas an E. coli-E. coli co-cultivation study have reported the production of 2 g/L CCM. Though tolerance to low-pH fermentations should make yeast an economically feasible chassis for biobased production of dicarboxylic acids like CCM, the CCM biosensor design based on BenM may need to be adjusted or evolved as production hosts become better and the product titers gets higher. Additionally, the biosensor will need to be matched to the production kinetics of the individual strain or library of biocatalysts.

Nevertheless, the LTTR-based ligand-inducible transcriptional activators reported here are much-needed tools for optimizing biocatalysts that produce chemicals and fuels for which there exist no high-throughput screen or selection. This should spur interest in developing many other orthogonal logic gates based on LTTR members, which could serve as a vast and valuable reservoir for developing new ligand-inducible genetic circuits capable of high-throughput screening, reprogramming and growth-coupled strain selection for bio-based production of chemicals. Furthermore, as the mode-of-action of transcriptional activators (YES) differ from that of repressors (NOT), the future possibility for higher-order designs within cellular reprogramming can now be exploited in greater diversity.

### SUMMARY OF THE INVENTION

It has been found by the present inventor(s) that transcriptional activators from prokaryotic organisms may work in a eukaryotic chassis by the positioning of the operator at a particular place within the eukaryotic promoter. The inventors found that this can be used e.g. for providing biosensors for intracellular and extracellular accumulation of a ligand or metabolites produced within a eukaryotic cell.

So, in a first aspect the present invention relates to a eukaryotic cell, such as a yeast cell comprising a bacterial transcriptional activator and a corresponding operator sequence positioned in a eukaryotic promoter, such as positioned within an endogenous promoter of said cell, which activator controls the expression of a gene from said eukaryotic promoter.

In a second aspect the present invention relates to a eukaryotic cell, such as a yeast cell comprising a bacterial transcriptional activator and a corresponding operator sequence, which activator controls the expression from a eukaryotic promoter, such as an endogenous promoter of the cell in response to a ligand specifically binding the transcriptional activator.

In a third aspect the present invention relates to a eukaryotic cell, such as a yeast cell comprising a bacterial transcriptional activator and a corresponding operator sequence positioned in a eukaryotic promoter, such as positioned within an endogenous promoter of the cell, which activator controls the expression of a gene from the eukaryotic promoter depending on the presence, such as dose dependent, of a ligand specifically binding the transcriptional activator

In a further aspect the present invention relates to the use of a prokaryotic transcriptional activator as a regulator of transcription in a eukaryotic cell, such as a yeast cell according to the invention; the transcriptional activator being activated by a ligand specifically binding the transcriptional activator to induce the expression of a protein product from a eukaryotic promoter of the cell; the promoter containing the operator sequence corresponding to the transcriptional activator.

In a further aspect the present invention relates to a the use of a prokaryotic transcriptional activator as a regulator of transcription in a eukaryotic cell, such as a yeast cell according to the invention; the transcriptional activator being activated depending on the presence, such as dose dependent, of a ligand specifically binding the transcriptional activator to induce the expression of a protein product from a eukaryotic promoter of said cell, the promoter containing the operator sequence corresponding to said transcriptional activator positioned within the promoter.

In a further aspect the present invention relates to a the use of a prokaryotic transcriptional activator as a metabolite biosensor for measuring the amount of a ligand extracellular of and/or produced by a eukaryotic cell, such as a yeast cell according to the invention, wherein the ligand specifically bind the transcriptional activator to induce expression of a reporter gene from a eukaryotic promoter of the cell, the promoter containing the operator sequence corresponding to the transcriptional activator.

In a further aspect the present invention relates to a method for measuring the amount of a ligand intracellular or extracellular of a eukaryotic cell, such as a yeast cell; said cell comprising a bacterial transcriptional activator and a corresponding operator sequence, which activator controls the expression of a reporter gene from a eukaryotic promoter of said cell in response to said ligand specifically binding said transcriptional activator; said promoter containing the operator sequence corresponding to said transcriptional activator; said method including the steps of

a) Cultivating a eukaryotic cell according to the invention;
b) Measuring the output from said promoter of said reporter gene;
c) Correlating said output from step b) with amount of said ligand.

In some embodiments the ligand is not produced by the eukaryotic cell, but is present in a solution of the cultivation medium of the eukaryotic cell, or such as when used to report toxic waste in a soil.

In a further aspect, the present invention relates to a recombinant transcriptional activator with increased activity, such as BenM with mutations at any one or more of the positions H110R, F211V, and Y286N.

### LEGENDS TO THE FIGURE

FIG. 1. Onboarding the cis-cis-muconic acid (CCM) responsive prokaryotic transcriptional activator BenM in yeast. (a) Schematic outline of native and synthetic full-length (491 bp) CYC1 promoter variants with different BenO positioning and number (T1 and/or T2). The transcriptional activator BenM from Acinetobacter sp. ADP1 controls expression of GFP from the synthetic CYC1 promoter with BenM operator (BenO) integrated at position T1 and/or T2. CCM further induces BenM-dependent expression of GFP (b) Mean fluorescence intensity (MFI) values from flow cytometry measurements of GFP intensities in the presence or absence of BenM expressed from the constitutively active TEF1 promoter, and following 24 h of incubation in the presence or absence of 1.4 mM CCM. (c) Screening 84 yeast strains expressing all possible combinations of BenM expression levels (TDH3p, TEFp, RNR2p and REV1p) individually or in combination with native or engineered CYC1p reporter promoters of different lengths (491 bp, 272 bp, 249 bp and 209 bp), BenO positioning, and number (T1 and/or T2) by flow cytometry. Outputs are ordered according to GFP intensity in control medium. Dashed lines indicate background fluorescence as inferred from BenM expressing strains without GFP, and arrow indicate best-performing biosensor design. Genotypes and GFP expression levels of all 84 strains are listed in Tables 1 and 2, respectively. (d) Heat-maps showing fold change (FC) in GFP expression between CCM-induced and control cultures of 80 strains shown in (c). For (b) and (c) mean fluorescence intensities (MFI) are shown as mean±s.d. from three (n=3) biological replicate experiments. AU, arbitrary units.

FIG. 2. High-throughput engineering and screening of BenM variants with improved CCM-inducibility. (a) Purified products from three rounds of error-prone PCR (epPCR) using the effector-binding domain (EBD) of BenM as template, were co-transformed into yeast together with a linearized centromeric plasmid, to allow for in vivo library reconstitution by gap repair and expression of wild-type BenM DNA binding domain (DBD) fused to approx. 40,000 variants of the EBD. Transformed yeast contained a chromosomal integration of 209 bp_CYC1p_BenO_T1 controlling the expression of GFP to allow for FACS-based screening of BenM variants with improved CCM-inducibility. (b) Representative flow cytometry histograms of fluorescence intensities obtained from a population of yeast cells expressing CCM sensor variants in control (grey) and CCM-inducer (light green) media. Control, CCM-induced and sorted (darker green) cell populations are normalized to mode for comparison. The proportions of cells within each histogram were calculated by FlowJo software as described in Methods (c) Isolated BenM variants were grown as clonal cultures, validated by flow cytometry and the EBDs of variants with significantly higher GFP expression under CCM-induced cultivation were sequenced. Mean fluorescence intensities (MFI) are shown as mean±s.d. from three (n=3) biological replicate experiments. AU, arbitrary units. (d) Ribbon representations of the EBD of BenM (PDB 2F7A) with the residue changes identified in BenM^{H110R, F211V, Y286N} highlighted in green. Bound CCM is highlighted as a magenta Van der Waals sphere.

FIG. 3. Biosensor specificity and transcriptional orthogonality. (a) The specificity of the CCM biosensor was tested by addition of various dicarboxylic acids (1.4 mM) to the growth medium. GFP expression was measured by flow cytometry following 24 h of cultivation. (b) RNA sequencing FPKM (fragments per kilo base per million) are plotted for yeast cells stably expressing 209 bp_CYC1p_BenO_T1::GFP reporter construct and BenM^{H110R, F211V, Y286N} versus cells only expressing the reporter construct following 24 h of cultivation in medium supplemented with CCM. Purple area indicates 2-fold cut-off and red dots significantly differentially regulated genes as inferred from cuffdiff (>2-fold, P<0.05)(see also FIG. 5). All data points are averaged from three (n=3) biological replicates.

FIG. 4. Onboarding transcriptional activators from the LTTR family as biosensors in yeast. (a) Left: Schematic illustration of LTTR-mediated activation of GFP expression by binding to the cognate operator in position T1 of 209 bp_CYC1p. Right: The 209 bp_CYC1p_T1 reporter promoter design supports GFP expression when controlled by individual LTTR transcriptional activators expressed from either a weak (REV1p) or a strong (TDH3p) promoter. The y-axis shows fold induction in mean fluorescence intensity (MFI) in cells expressing individual LTTRs relative to cells not expressing the LTTRs. (b) Left: Schematic illustration on external application of individual ligands for induction of LTTR-mediated activation of GFP expression. Right: External application of individual ligands can induce LTTR-mediated activation of GFP expression. The y-axis shows fold induction in mean fluorescence intensity (MFI) for cells grown for 24 h in medium containing either cis, cis-muconic acid (CCM), naringenin (NAR), L-arginine (ARG), protocatechuic acid (PCA) or malonic acid (MAL) compared to cells growing in control medium. (c) Heatmap showing orthogonality of MdcR- and ArgP-mediated transcriptional regulation of GFP expression controlled by either reporter promoter 209 bp_CYC1p_MdcO_T1 or 209 bp_CYC1p_ArgO_T1 (Table 4). Color key shows mean fluorescence intensity (MFI) from three (n=3) biological replicate experiments. For (a) and (b), mean fluorescence intensity (MFI) values and their error bars are calculated as mean±s.d. from three (n=3) biological replicates.

FIG. 5. Biosensor sensitivity and operational range. (a) The response functions of wild-type and engineered BenM^{H110R, F211V, Y286N} expressed in yeast from REV1p as measured by flow cytometry using various concentrations of CCM (24 h) and the 209 bp_CYC1p_BenO_T1 promoter controlling the expression of GFP. A yeast strain without BenM expressed is used as a control for background GFP fluorescence from the 209 bp_CYC1p_BenO_T1 promoter. (b) The response function measurement for the naringenin biosensor when FdeR is expressed from a weak (REV1p) or a strong (TDH3p) promoter using various concentrations of naringenin (24 h) and the 209 bp_CYC1p_FdeO_T1 reporter promoter controlling the expression of GFP. A yeast strain without FdeR expressed is used as a control for background GFP fluorescence from the 209 bp_CYC1p_FdeO_T1 promoter. For (a) and (b) mean fluorescence intensity (MFI) values and their error bars are calculated as mean±s.d. from three (n=3) biological replicate experiments

FIG. 6. In vivo application of CCM and naringenin biosensors in yeast. (a) Schematic representation of the heterologous 3-step CCM production pathway for testing BenM as a biosensor for in vivo CCM production in yeast. Additionally, over-expression of Tkl1 was included together with balancing of the heterologous three-step pathway (PaAroZ, KpAroY and CaCatA) using single or multi-loci integration of AroY subunits B and C (Iso, isoform)(see Methods and Table 1). (b) Following 24 h of cultivation, CCM titers and MFIs were quantified and plotted for each strain. (c) Schematic representation of heterologous 5-step naringenin production pathway adopted from Naesby et al. For the hydroxylation of cinnamate to coumarate a fusion protein of AtC4H and AtATR2 was used. For testing FdeR as a biosensor for in vivo naringenin production in yeast, mean fluorescence intensity (MFI) in three different strains engineered with one copy of the 5-step naringenin production pathway (EVR1) or with one (EVR2) or two (EVR3) additional integrations of bottleneck enzymes were compared to a control strain (EVR0, ctrl) without the production pathway. Following 48 h of cultivation, naringenin titers and MFIs were quantified and plotted. For both (b) and (d) data are average of three biological replicates. Mean fluorescence intensity (MFI) values and metabolite quantifications are presented as means±s.d. from three (n=3) biological replicate experiments.

FIG. 7 BenM regulation of the ben operon during benzoate catabolism in Acinetobacter sp. ADP1 is feed-back induced by the intermediate catabolite cis,cis-muconic acid (CCM). Upon detection of CCM from the aromatic acid catabolism, the constitutively DNA-bound BenM tetramer undergoes a conformational change facilitating the accessibility of RNA polymerase and active transcription of the ben operon (Bundy, B. M., Proc. Natl. Acad. Sci. U.S.A 99, 7693-8 (2002). Sequence of the BenM operator (BenO). The three potential binding sites for BenM are highlighted in blue with site 1 displaying dyad symmetry exactly matching the consensus sequence of LysR-type regulators (Collier, L. S., 3. Bacteriol. 180, 2493-501 (1998)).

FIG. 8 (a) Sequence outline of the full-length CYC1 promoter with native upstream activating sequences (UASs) shown in blue and operator sites for TATA-binding proteins shown in red. Sites for positioning of BenM operators are marked with black triangles (T1 and T2) and sites for truncations (272 bp, 249 bp and 209 bp) marked with dashed vertical lines. USER cloning site and Kozak sequence is italicized upstream the open reading frame of yeast-enhanced GFP (bold). (b) Sequence of the FdeR, PcaQ, ArgP and McdR operators used to swap into position T1 of the 209 bp_CYC1p shown in (a) (Siedler, S., Metab. Eng. 21, 2-8 (2014) and Maclean, A. M., Microbiology 157, 2522-33 (2011).

FIG. 9 (a) Uptake of cis,cis-muconic acid (CCM) at pH 4.5 by S. cerevisiae cells following 24 h of growth. (b) Representative growth curves of yeast cells in liquid medium containing different concentrations of CCM. OD values were determined at 1-h intervals over 25-h period. For both (a) and (b) data display means±s.d. from three (n=3) biological replicate cultivations.

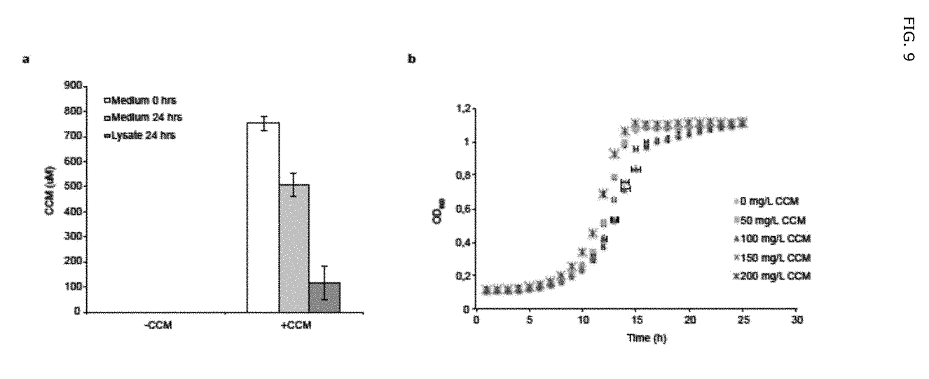

FIG. 10 Screening 84 yeast strains expressing all possible combinations of BenM expression levels (TDH3p, TEFp, RNR2p and REV1p) individually or in combination with native or engineered CYC1p reporter promoters of different lengths (491 bp, 272 bp, 249 bp and 209 bp), BenO positioning, and number (T1 and/or T2) by flow cytometry after 24 h of growth in control medium or medium supplemented with 1.4 mM CCM. Outputs are ordered according to GFP intensity in control medium. Dashed lines indicate background fluorescence as inferred from strains expressing only BenM (no reporter). Genotypes and GFP expression levels of all 84 strains can be found in Tables 1 and 2, respectively. Mean fluorescence intensity (MFI) values and their error bars are calculated as mean±s.d. from three (n=3) biological replicate experiments. AU, arbitrary units.

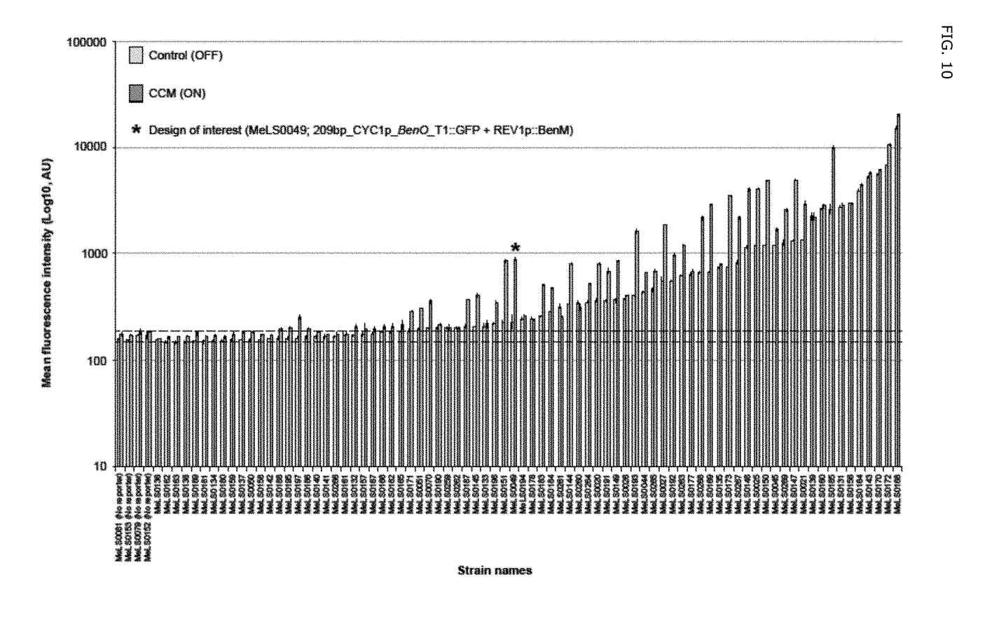

FIG. 11 (a) A box and whisker plot showing the mean value, 1 and 99 percentiles for three (n=3) biological replicate RNA sequencing experiments. Outliers are depicted as black dots. The GFP is highlighted in green. RNA was collected following 24 h of cultivation in mineral medium pH 4.5 with 1.4 mM CCM. The fold change in FPKM is displayed as a log 2 normalized value for all expressed genes. (b) A fold change histogram of FPKM showing the fold changes (log 2) in gene expression from FIG. 2c plotting strain including BenMH110R, F211V, Y286N (MeLS0284) over the strain without BenMH110R, F211V, Y286N (MeLS0138). GFP is indicated with an arrow. The data are representative of three (n=3) biological replicates.

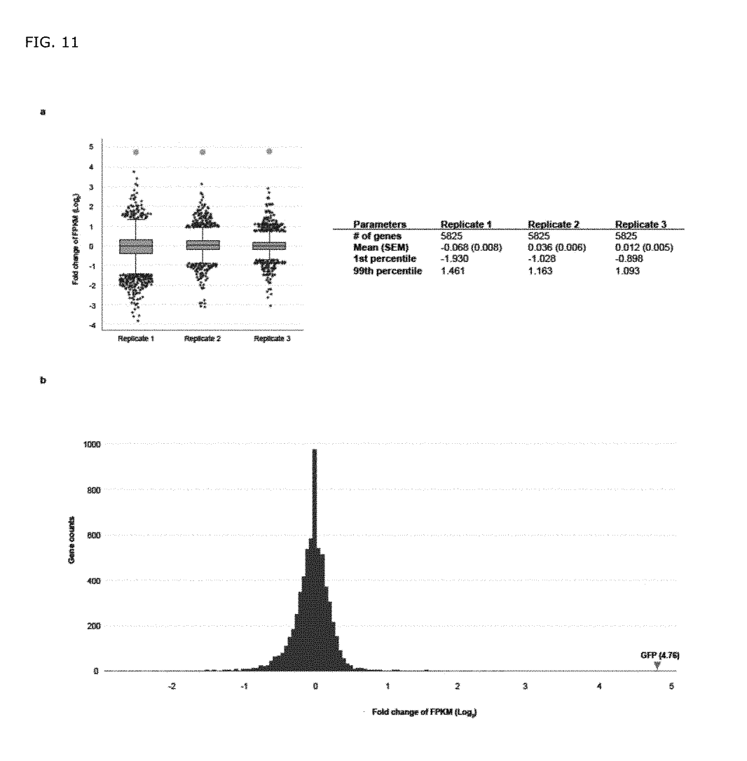

FIG. 12 Endogenous response function of the CCM and naringenin biosensors. (a) Cultivation medium was analyzed for CCM concentration by LC-MS and flow cytometry performed for GFP intensity measurements of six different CCM producing strains compared to a reference CCM null background strain (see Table 1) following 24 h and 72 h cultivations. (b) Average titers for the six CCM-producing strains at 24 h and 72 h of cultivation, compared to the reference strain. (c) Cultivation medium was analyzed for naringenin concentration by UPLC and flow cytometry performed for GFP intensity measurements of three different naringenin producing strains compared to a reference naringenin null background strain following 24 and 48 h cultivations. (d) Average titers for the three naringenin-producing strains at 24 h and 48 h of cultivation, compared to the reference strain. For (a-d) data are presented as means±s.d. from three (n=3) biological replicate experiments. Table 1 lists all strain genotypes.

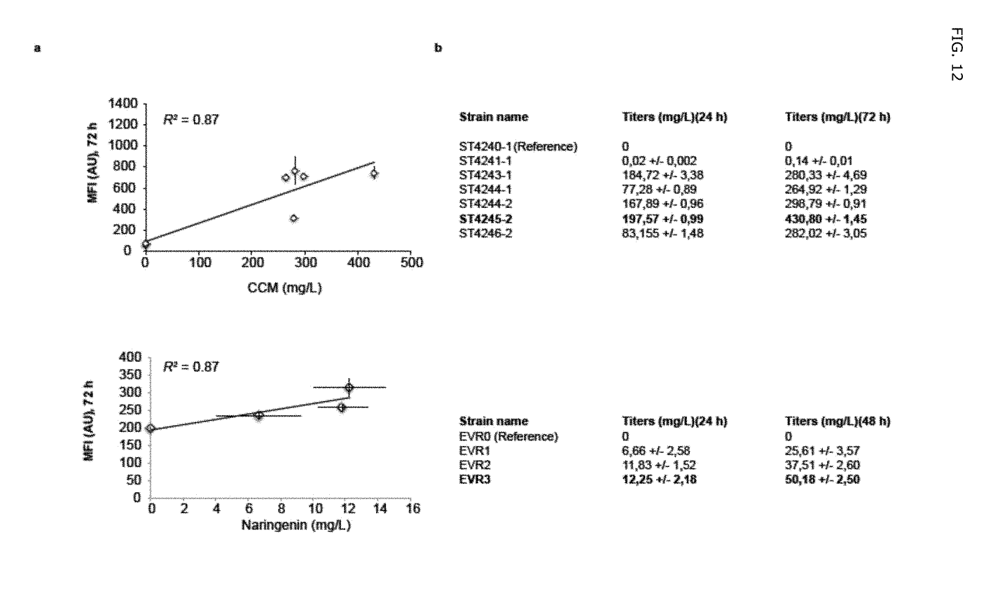

FIG. 13. BenM activates reporter expression in CHO. CHO cells were transfected with a plasmid with the BenO-containing human cytomegalovirus (CMV) promoter controlling the expression of GFP as well as an empty vector (− BenM) or a vector expressing BenM (+ BenM). Total GFP expression was measured after 24 h, and normalized by total RFP expression. Average and standard deviation are based on three biological replicates. *; p<0.05 (t-test).

FIG. 14. Screening 17 yeast strains expressing BenM from the REV1p in combination with CYC1p reporter promoter of 209 bp with BenO placed at different positions upstream of TATA1. The fold induction (mean±s.d.) was calculated by dividing mean fluorescence intensity (MFI) in medium with 1.4 mM CCM by the MFI in control medium as measured by flow cytometry after 24 h of growth for three biological replicates.

### DETAILED DISCLOSURE OF THE INVENTION

### Definitions

The term “eukaryotic cell” is used herein in its normal sense. The term includes any animal, mammalian, fungi, yeast, insect and algae cell. In some specific embodiments the eukaryotic cell is a yeast cell.

The term “yeast cell” refers to the single-celled microorganisms classified as members of the fungus kingdom. The term includes but is not limited to cells of a genus selected from the group consisting of Kluyveromyces, Saccharomyces and Hanensula, such as a yeast cell selected from the group consisting of Saccharomyces cerevisiae and Saccharomyces boulardii.

The term “bacterial transcriptional activator” as used herein refers to a protein, such as any know protein naturally derived from a bacterium (a transcription factor) that increases gene transcription of a gene or set of genes in this bacterium. It is to be understood that a bacterial transcriptional activator and its corresponding operator sequence (and whether it is derived from a prokaryote genome and not found in a eukaryotes motif) will be easily identified by a simple sequence search for the person skilled in the art. Essentially, for a transcriptional regulator to be defined as a prokaryotic transcriptional activators by the person skilled in the art it must adhere to all the following points:

a) The gene encoding the protein sequence is found natively in a prokaryotic genome.
b) When using the Basic Local Alignment Search Tool (BLAST; https://blast.ncbi.nlm.nih.gov/Blast.cgi) for comparing nucleotide or protein sequences to sequence databases, the query sequence aligns more to sequences of prokaryote origin in terms evolutionary relationships than to sequences of eukaryote origin.
c) In its native context of a prokaryotic genome, deletion of the gene encoding the protein sequence of the transcriptional regulator will cause lower or no change in expression of its target gene.
d) In its native context of a prokaryotic genome, over-expression of the gene encoding the protein sequence of the transcriptional regulator will not cause lower expression of its target gene, as is the case for transcriptional repressors.
e) The gene encoding the protein sequence is categorized functionally as an activator in the RegPrecise database (http://regprecise.lbl.gov); a database of curated genomic inferences of transcriptional regulatory interactions in prokaryotes.

In some embodiments the bacterial transcriptional activator is within the prokaryote super-family of LysR-type transcriptional regulators (LTTRs).

In some embodiment the bacterial transcriptional activator as used herein is selected from the list of tables 6 and 7.

In some embodiments the term refers to an intact transcriptional activator containing both an activation domain and a DNA-binding domain.

The term “corresponding operator sequence” as used herein refers to the DNA sequence that binds a specific bacterial transcriptional activator in order to make the bacterial transcriptional activator take effect. The operator sequence is placed within the promoter on which the activator works. The corresponding operator sequence is identified as part of the DNA sequence of a prokaryote promoter sequence, which is under the regulation of the bacterial transcriptional activator.

The term “eukaryotic promoter” as used herein refers to a region of DNA derived from or within a eukaryotic cell that initiates transcription of a particular gene downstream of this promoter.

The term “endogenous promoter” as used herein refers to a promoter that normally is present in the cell in use.

The term “ligand specifically binding a transcriptional activator” as used herein refers to a ligand, which specifically binds to a particular transcriptional activator to control the functioning of the activator in a system of a so-called ligand-inducible transcriptional regulator.

The term “exogenous” refers to a gene that originates outside of the organism of the specific cell being used.

In some embodiments, the eukaryotic cell contains a reporter gene, preferably in operative linkage with the eukaryotic promoter responsive to the bacterial transcriptional activator. Exemplary reporter genes include enzymes, such as luciferase, phosphatase, or p-galactosidase which can produce a spectrometrically active label, e. g., changes in color, fluorescence or luminescence. In some embodiments the reporter gene encodes a gene product selected from the group consisting of luciferase, green fluorescent protein, p-lactamase chloramphenicol acetyl transferase, ss-galactosidase, secreted alkaline phosphatase, p-lactamase, p-glucuronidase, alkaline phosphatase, blue fluorescent protein, and chloramphenicol acetyl transferase.

“Upstream activating sequences” as used herein refers to cis-acting elements of a eukaryotic promoter that modulate the rate of initiation of transcription well known to the person skilled in the art. Specific sequence and number of subsites or regions is specific for the promoter being used.

### Specific Embodiments of the Invention

As described above the present invention relates to a eukaryotic cell, such as a yeast cell comprising a ligand-binding bacterial transcriptional activator and a corresponding operator sequence positioned in a eukaryotic promoter, such as positioned within an endogenous promoter of the cell, which activator controls the expression of a gene from the eukaryotic promoter.

In some embodiments the expression of a gene from said eukaryotic promoter is depending on the presence, such as dose dependent, of a ligand specifically binding the transcriptional activator.

In some embodiments the cell comprises a gene encoding the expression of the ligand, one or more genes encoding a pathway of enzymes synthesizing the ligand, and/or a gene encoding a compound that is metabolized into the ligand. In some embodiment such a gene is expressed from the eukaryotic promoter.

In some embodiments the cell comprises an exogenous reporter gene, and/or one or more further regulatory gene, such as a gene encoding antibiotic resistance. In some embodiment such a gene is expressed from the eukaryotic promoter.

In some embodiments the reporter gene provides for fluorescence output, such as a gene encoding green fluorescent protein, blue fluorescent protein or luciferase.

In some embodiments the one or more the genes independently selected from the gene encoding the expression of the ligand, one or more genes encoding a pathway of enzymes synthesizing the ligand, a gene encoding a compound that is metabolized into the ligand, an exogenous reporter gene, and one or more further regulatory gene; is under the control and/or is activated by the eukaryotic promoter.

In some embodiments the transcriptional activator is selected from any one selected from table 6, such as any one selected from BenM, FdeR, MdcR, and ArgP.

In some embodiments the ligand and transcriptional activator is selected from muconic acid and BenM; Naringenin and FdeR; Malonate and MdcR, and L-arginin and ArgP.

In some embodiments the cell is a yeast cell, such as Saccharomyces cerevisiae.

In some embodiments the cell is a mammalian cell, such as a Chinese hamster ovary cell.

In some embodiments the promoter is a full length promoter, or a truncated version with upstream activating sequences, such as UAS1 and UAS2 of the CYC promoter, removed.

In some embodiments the promoter is a yeast promoter, such as the full length CYC1 promoter or CYC1 with upstream activating sequences (UAS1 and UAS2) removed.

In some embodiments the promoter is a mammalian promoter, such as the full length CMV promoter.

In some embodiments the transcriptional activator work through a promoter-centric mechanism, wherein the transcriptional activator bind to an operator site in the promoter thereby improving its ability to guide RNA polymerase to initiate transcription.

In some embodiments the transcriptional activator does not require binding to any other regulatory subunits and/or which cell is without any further engineering or the co-expression of other molecular components regulating the transcriptional activator.

In some embodiments the transcriptional activator does not require binding to any other regulatory subunits apart from its specific ligand and/or which cell is without any further engineering or the co-expression of other molecular components regulating said transcriptional activator.

In some embodiments the operator sequence is specific for the transcriptional activator within the promoter.

In some embodiments the operator sequence is positioned immediately upstream of the TATA box, such as a TATA box 1, such as TATA-1β, such as anywhere between 6-15 bp, such as anywhere between 6-14 bp, such as anywhere between 6-13 bp, such as anywhere between 6-12 bp, such as anywhere between 6-11 bp, such as anywhere between 6-10 bp, such as anywhere between 6-9 bp, such as anywhere between 6-8 bp, such as anywhere between 6-7 bp, such as 6 bp upstream of said TATA box of said eukaryotic promoter.

In some embodiments the operator sequence is positioned immediately upstream of one of the two TATA boxes—TATA-1β, such as anywhere between 6-15 bp upstream of TATA box 1, such as anywhere between 6-14 bp upstream of TATA box 1, such as anywhere between 6-13 bp upstream of TATA box 1, such as anywhere between 6-12 bp upstream of TATA box 1, such as anywhere between 6-11 bp upstream of TATA box 1, such as anywhere between 6-10 bp upstream of TATA box 1, such as anywhere between 6-9 bp upstream of TATA box 1, such as anywhere between 6-8 bp upstream of TATA box 1, such as anywhere between 6-7 bp upstream of TATA box 1, such as 6 bp upstream of TATA box 1.

In some embodiments the operator sequence is positioned immediately 6 bp upstream of the TATA box—TATA-1β, such as anywhere between 6-15 bp upstream of TATA box 1, such as anywhere between 6-14 bp upstream of TATA box 1, such as anywhere between 6-13 bp upstream of TATA box 1, such as anywhere between 6-12 bp upstream of TATA box 1, such as anywhere between 6-11 bp upstream of TATA box 1, such as anywhere between 6-10 bp upstream of TATA box 1, such as anywhere between 6-9 bp upstream of TATA box 1, such as anywhere between 6-8 bp upstream of TATA box 1, such as anywhere between 6-7 bp upstream of TATA box 1, such as 6 bp upstream of TATA box 1.

In some embodiments the transcriptional activator belongs to the prokaryote super-family of LysR-type transcriptional regulators (LTTRs).

In some embodiments the operator is an LTTR operator sequence selected from BenO, FdeO, MdcO, and ArgO.

In some embodiments the transcriptional activator is co-expressed in the cell, such as from a promoter selected from TEF1, REV1, RNR2 and TDH3.

In some embodiments the transcriptional activator is a functional variant with increased activity, such as BenMH110R, F211V, Y286N.

### Example 1

Strains, chemicals and media. Saccharomyces cerevisiae CEN.PK102-5B (MATa ura3-52 his3Δ1 leu2-3/112 MAL2-8^{c} SUC2), CEN.PK113-5A (MATa, trp1 his3Δ1 leu2-3/112 MAL2-8^{c} SUC2) and CEN.PK113-7D (wild type, MATa MAL2-8^{c} SUC2) strains were obtained from Peter Kotter (Johann Wolfgang Goethe-University Frankfurt, Germany). In principal any other yeast strains may be used, such as one obtained from public repository EuroScarf. EasyClone plasmids used in this work are described in Jensen, N. B. et al. EasyClone: method for iterative chromosomal integration of multiple genes in Saccharomyces cerevisiae. FEMS Yeast Res. 14, 238-48 (2014). Escherichia coli strain DH5a was used as a host for cloning and plasmid propagation. The chemicals and Pfu TURBO DNA polymerase were commercially obtained (Sigma-Aldrich and Agilent Technologies Inc., respectively). All acids used were >97% in purity. S. cerevisiae cells were grown at 30° C. in synthetic complete medium as well as drop-out media and agar plates were prepared using pre-mixed drop-out powders (Sigma-Aldrich). Mineral medium was freshly prepared as described previously. For all media containing diacids, 1.4 mM of the individual diacids were dissolved in mineral medium and pH adjusted to 4.5 before sterile filtration. For CCM several dilutions were made to examine the performance of the CCM biosensor. For naringenin, mineral medium was supplemented with 0, 0.05, 0.10 or 0.20 mM naringenin, dissolved in ethanol, the final ethanol concentration for each medium was 2% (v/v), and the final pH of the medium was adjusted to 6.0. E. coli cells were grown at 37° C. in Luria-Bertani (LB) medium supplemented with 100 μg/mL ampicillin.

Synthetic Genes and Oligonucleotides.

Oligonucleotides and synthetic genes were commercially synthesized (Integrated DNA Technologies, Inc. and Thermo Fisher Scientific Inc., respectively). Sequences of synthetic genes and oligonucleotides can be found in Tables 4 and 5, respectively.

Plasmids, Strains and Library Construction.

Except Arabidopsis thaliana At4CL-2 (NM 113019.3) and Saccharomyces cerevisiae ScTKL1 (NM_001184171.1), all genes encoding Klebsiella pneumoniae AroY.B (AAY57854.1), AroY.Ciso (BAH20873.1), AroY.D (AAY57856.1), Candida albicans CatA (XP_722784.1), Podospora anserina AroZ (XP_001905369.1), Acinetobacter sp ADP BenM (AAC46441.1), Arabidopsis thaliana AtC4H (NM_128601.2), Arabidopsis thaliana AtATR2 (NM_179141.2), Arabidopsis thaliana AtPAL2 (NM_115186.3), Petunia hybrida PhCHI (X14589), Hypericum androsaemum HaCHS (AF315345), Schizosaccharomyces pombe MAE1 (NM_001020205.2), Sinorhizobium meliloti PcaQ (NC_003078.1), Escherichia coli ArgP (NC_000913.3), Klebsiella pneumonia MdcR (U14004), and Herbaspirillum seropedicae SmR1 FdeR (Hsero_1002, UniProtKB-D8J0W4_HERSS) were codon-optimized for expression in yeast (see Table 4 for full sequences). All gene fragments and correct overhangs for USER-cloning were amplified by PCR using oligonucleotides listed and described in Table 5. Unless otherwise stated the amplified products were USER cloned into EasyClone integrative plasmids Jensen, N. B. et al. (2014), and confirmed by sequencing.

The list of the constructed plasmids can be found in Table 3. Transformation of yeast cells was carried out by the lithium acetate method Gietz, R. D. & Schiestl, R. H. Large-scale high-efficiency yeast transformation using the LiAc/SS carrier DNA/PEG method. Nat. Protoc. 2, 38-41 (2007), and strains selected on synthetic drop-out medium (Sigma-Aldrich), selecting for appropriate markers. For selection of strain carrying KanMX and HypMX, the media was supplemented with 200 μg/mL G418 sulphate and 200 μg/mL hygromycin B, respectively. Transformants were genotyped using oligonucleotides described in Table 5. The resulting strains are listed in Table 1.

To establish the CCM producing strains, we expressed the dehydroshikimate DHS dehydratase from P. anserina (PaAroZ), the PCA decarboxylase genes from K. pneumoniae (KpAroY.B, KpAroY.Ciso, KpAroY.D), and the catechol 1, 2 dioxygenase CDO from Candida albicans (CaCatA) in S. cerevisiae. It has been reported that the conversion of PCA to catechol by PCA decarboxylase is a limiting step. For this reason we expressed the KpAroY.B and KpAroY.Ciso genes in either single or multiple genomic integrations to create a small library of CCM production strains. In addition, Tkl1 was overexpressed in order to improve the precursor supply.

To establish a naringenin producing strain we integrated the full pathway containing the phenylalanine ammonium lyase from Arabidopsis thaliana (AtPAL-2), the fusion of cinnamate 4-hydroxylase from Arabidopsis thaliana and NADPH-cytochrome P450 reductase from Arabidopsis thaliana (AtC4H:L5:AtATR2), the 4-coumarate-CoA ligase 2 from Arabidopsis thaliana (At4CL-2), the naringenin-chalcone synthase from Hypericum androsaemum (HaCHS), and the chalcone isomerase from Petunia hybrida (PhCHI) to make strain EVR1 (table 1). Strains EVR2 and EVR3 contained one and two additional integrations of bottleneck enzymes (AtPAL-2 and HaCHS for EVR2; AtPAL-2, HaCHS, and AtC4H:L5:AtATR2 for EVR3)(table 1).

Mutagenesis of BenM and Library Preparation.

For optimization of the CCM inducibility of BenM, purified products from three consecutive rounds of error-prone PCR (epPCR) of the effector-binding domain (EBD, residues 90-304) of BenM, were co-transformed into yeast together with linearized centromeric plasmid according to Eckert-Boulet et al. (Eckert-Boulet, N., Pedersen, M. L., Krogh, B. O. & Lisby, M. Optimization of ordered plasmid assembly by gap repair in Saccharomyces cerevisiae. Yeast 29, 323-34 (2012)), to allow for in vivo gap repair and library reconstitution of wild-type BenM DNA binding domain (DBD) fused to EBD variants expressed from REV1p. For epPCR we used the GeneMorph II kit according to manufacturer's description (Agilent Technologies). Transformed yeast contained a chromosomal integration of the 209 bp_CYC1p_BenO_T1 promoter controlling the expressing of GFP at EasyClone site 4 on chromosome XII (Mikkelsen, M. D. et al. Microbial production of indolylglucosinolate through engineering of a multi-gene pathway in a versatile yeast expression platform. Metab. Eng. 14, 104-11 (2012)), to allow for FACS-based screening of improved CCM-inducible BenM variants.

Metabolite Quantification Using HPLC and UPLC-MS.

The CCM production strains were cultivated in 24-deep well plate with air-penetrable lids (EnzyScreen) to test for the production of CCM. Colonies from the individual strain were inoculated in 1 mL synthetic drop-out medium (Sigma-Aldrich), selecting for URA, HIS and LEU markers, and grown at 30° C. with 250 rpm agitation at 5 cm orbit cast for 24 h. 300 μL of the overnight cultures were used to inoculate 3 mL mineral medium (pH 4.5) in 24-deep well plate and incubated for 24-72 h at the same conditions as above. Experiments were performed in triplicates. The culture broth was centrifuged 3,500×rpm and the supernatant analyzed for CCM concentration using HPLC. For this purpose, samples were analyzed for 45 min using Aminex HPX-87H ion exclusion column with a 1 mM H₂SO₄ flow of 0.6 mL/min. The temperature of the column was 60° C. Refractive index and UV detectors (Dionex) were used for detection of CCM (250 nm). CCM concentrations were quantified by comparison with the spectrum of the standards. For the naringenin production strains 300 μl culture broth was extracted with 300 μl MeOH in a 10-minute incubation (300×rpm, 5 cm amplitude, 30° C.) in a 96 square deep-well microtiter plate (Greiner Masterblock, 96 Well, 2 ml, P, V-bottom) and subsequently clarified by centrifugation at 4000×g for 5 min. Clarified broth extract was subsequently diluted four times with 50% MeOH and 2 μl was injected on a Acquity UPLC system (Waters) coupled to a Acquity TQ mass detector (Waters). Separation of the compounds was achieved on a Acquity UPLC® BEH C18 column (Waters, 1.7 μm, 2.1 mm×50 mm), kept at 55° C. Mobile phases A and B were water containing 0.1% formic acid and acetonitrile containing 0.1% formic acid, respectively. A flow of 0.6 ml/min was used. The gradient profile was as follow: 0.3 min constant at 10% B, a linear gradient from 10% B to 25% B in 3.7 min, a second linear gradient from 25% B to 100% B in 1 min, a wash for 1 min at 100% B and back to the initial condition of 10% B for 0.6 min. The mass analyzer was equipped with an electrospray (ESI) source and operated in negative mode. Capillary voltage was 3.0 kV; the source was kept at 150° C. and the desolvation temperature was 350° C.; desolvation and cone gas flow were 500 L/h and 50 L/h respectively. EM-Hr ions of naringenin (271 m/z) was tracked in SIR mode. Naringenin was quantified using a quadratic calibration curve with authentic standards ranging from 0.039 mg/I to 20 mg/I using QuanLynx software (Waters).

Transport Assays.

Overnight grown CEN.PK113-5A cells were diluted (OD₆₀₀=0.1) into SC-HIS-LEU medium with or without 1.4 mM (200 mg/L) CCM. Media samples were taken at both 0 h and 24 h, while samples for measuring cellular lysates (10⁸ cells) were harvested at 24 h. For quantification of CCM by LC-MS, cultures were harvested by centrifugation. For extracellular CCM quantification the supernatant was centrifuged twice and filtered (0.2 μm) before analysis. For intracellular CCM quantification harvested pellets were washed twice in ice-cold isotonic saline solution (0.9% NaCl) and centrifuged at 5,000×g before cells were extracted in an aqueous 0.1% formic acid solution and sonicated for 15 min. Following this, samples were centrifuged at 13,000×g and supernatants filtered (0.2 μm) prior to analysis. LC-MS data was collected on EVOQ EliteTriple Quadrupole Mass Spectrometer system coupled with an Advance UHPLC (Bruker). Samples were held at 4° C. during the analysis. A 1 μL sample was injected onto a ACQUITY HSS T3 C18 UHPLC column (Waters), with a 1.8 μm particle size and 2.1×100 mm dimensioning. The column was held at a temperature of 30° C. The solvent system used was 0.1% formic acid (mobile phase A) and acetonitrile with 0.1% formic (mobile phase B). The flow rate was 0.400 ml/min with an initial solvent composition of 100% mobile phase A held until 0.50 min, then changed until it reached % A=5.0 and % B=95.0 at 1.00 min. This was held until 1.79 min when the solvent was returned to the initial conditions and the column was re-equilibrated until 4.00 min. The eluent was sprayed into the heated ESI probe of the MS which was held at 250° C. and a voltage of 2500 V. Sheath/nebulizer/cone gas flow rate of 50/50/20 units and cone temp was 350° C. Two transitions were chosen in negative Multiple Reaction Monitoring (MRM) mode for quantification of CCM: m/z 141.70-96.80 (quantification transition) and m/z 141.70-53.1 (confirmation transition). Triplicate measurements were made for all samples.

Fluorescence Activated Cell Sorting

A two-step method was used to sort for BenM variants that specifically induce in the presence of CCM. Cells (10× library size, approx. 400,000 cells) were inoculated in mineral medium without inducer and incubated for 24 h at 30° C., diluted into PBS, and then GFP intensity of individual cells was measured using a BD Biosciences Aria (Becton Dickinson) with a blue laser (488 nm) by applying tight gates on the FSC and SSC channels. Only cells displaying auto-fluorescence intensities were sorted in order to limit auto-activating BenM variants. Sorted cells were recovered in mineral medium, followed by subculturing (1:100) into mineral medium containing 1.4 mM (200 mg/L) cis,cis-muconic acid. The cells were grown for 24 h at 30° C., washed and subjected to a second round of FACS. Cells exhibiting high levels of GFP (top 1%) were sorted, recovered in mineral medium and plated for single colonies on SC-HIS-LEU media. Individual clones were subsequently validated using flow cytometry.

Flow Cytometry Measurements and Data Analysis.

Cells grown for 24 h in control (mineral medium, pH 4.5) or inducing medium (mineral medium pH 4.5+1.4 mM CCM, 1.4 mM protocatechuic acid, 10 mM malonic acid, 0.2 mM naringenin, or 50 mM L-arginine) were diluted into PBS to arrest cell growth. Cells were then analyzed by flow cytometry using a Fortessa flow cytometer (Becton Dickinson) with a blue laser (488 nm), for validation of single strains. For each strain 10,000 single-cell events were recorded. Events were analyzed using FlowJo software (TreeStar Inc.). The fluorescence arithmetic mean of the gated cell population was calculated, and the fold-change determined by dividing the mean fluorescence of the induced (ON) state with the mean fluorescence of the control (OFF) state. For flow cytometry for CCM and naringenin producing cells we tested mean fluorescence intensities from 10,000 cells pr. strain following 72 and 48 h, respectively. The data represent the average of three (n=3) biological replicates and error bars correspond to the standard deviation between these measurements.

Transcriptome Analysis.

To study the impact of ligand-induced BenM on genome-wide gene expression, triplicate cultures of strains MeLS0138 and MeLS0284 were grown for 24 h at 30° C. in 50 ml mineral medium pH 4.5 with 1.4 mM CCM. Following this, total RNA was extracted essentially as previously described (Kildegaard, K. R. et al. Evolution reveals a glutathione-dependent mechanism of 3-hydroxypropionic acid tolerance. Metab. Eng. 26, 57-66 (2014)). Briefly, 15 ml samples of the six cultures were harvested into a pre-chilled 50 ml tube with crushed ice and then immediately centrifuged at 4° C., 4000×rpm for 5 min. Subsequently, the pellets were resuspended in 2 ml of RNAlater® Solution (Ambion, Life Technologies) and incubated on ice for 1 h. Next, cells were pelleted by centrifugation (12,000×rpm for 10 s) and transferred to liquid nitrogen, and stored at −80° C. until further analysis. Total RNA extraction was performed using RNeasy® Mini Kit (QIAGEN). For this purpose, samples were thawed on ice, and 600 μl of buffer RLT containing 1% (v/v) 8-mercaptoethanol was added directly to the cells, before being transferred into a 2 ml extraction tube containing 500 μl glass beads and disrupted using the PRECELLYS®24 (Bertin Technologies) for 2×20 s at 6500×rpm. The cell mixture was pelleted and supernatant transferred to a new tube. Total RNA was purified according to the manufacturing's protocol, and genomic DNA removed using Turbo DNA-free™ Kit (Ambion). The quantity and quality of the RNA samples were measured using Qubit 2.0 Fluorometer using the Qubit RNA BR Assay (Thermo Fisher Scientific) and Agilent 2100 Bioanalyzer using the RNA 6000 Nano Kit (Agilent Technologies), respectively. For sequencing we used 3 μg of total RNA as input for TruSeq® Stranded mRNA Sample Preparation kit prior to sequencing on the MiSeq System using MiSeq Reagent Kit v3 150 cycles at a 2×75 bp read length configuration (Illumina) obtaining 38 M reads.

Bioinformatic Resources.

Two-dimensional heatmap plots were generated using the plot3D package and the R GUI. For ribbon structure representation of CCM-binding domain of BenM^{H110,F211V,Y286N} the UCSF Chimera software was used (Pettersen, E. F. et al. UCSF Chimera—a visualization system for exploratory research and analysis. 3. Comput. Chem. 25, 1605-12 (2004)). For RNA-seq data analysis, TopHat (2.0.13) and Cufflinks (2.2.1) suite was employed as previously described (Trapnell, C. et al. Differential gene and transcript expression analysis of RNA-seq experiments with TopHat and Cufflinks. Nat. Protoc. 7, 562-78 (2012)). Expression levels (Fragments Per Kilobase of exon per Million fragments mapped: FPKM) from three (n=3) biological replicates of the conditions tested are processed with cuffdiff to obtain fold change differences and to perform statistical testing. A q-value cutoff of <0.05 was used to identify genes that have significant differential expression. Additionally, a >2-fold cut-off selection criterion was applied. Reference genome and annotations for CEN.PK113-7D strain were retrieved from Saccharomyces Genome Database (SGD; http://www.yeastgenome.org/). Genes with FPKM=0 for any replicate were removed from consideration.

Database for RNA-Seq Data.

RNA-seq data are available in the ArrayExpress database (www.ebi.ac.uk/arrayexpress) under accession number E-MTAB-4836 (http://www.ebi.ac.uk/arrayexpress/experiments/E-MTAB-4836).

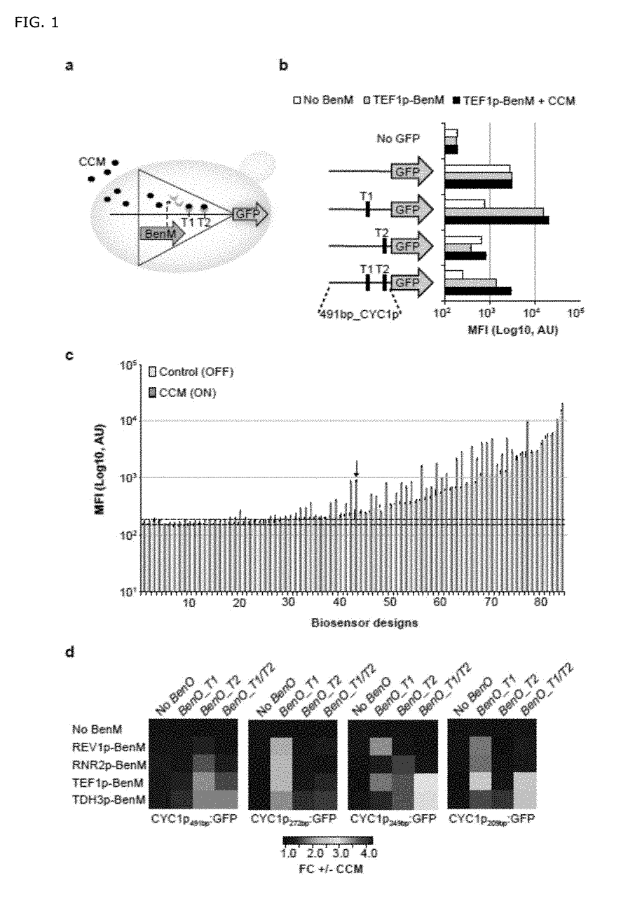

TABLE 1
Strain names and genotypes of all strains generated in this study. Strain names for those shown in
FIG. 1c are ordered according to basal activity (see also Supplementary Table 2)

| Strain | Yeast Integrative | |
|---|---|---|
| name | Plasmid (parent strain) | Genotype |
| CEN.PK113- | — | mat a URA3 HIS3 LEU2 TRP1 |
| 7D | | |
| CEN.PK102- | — | mat a ura3 his3 leu2 |
| 5B | | |
| Cen.PK113- | — | mat a his3 leu2 trp1 |
| 5A | | |
| MeLS0081 | pMeLS0045 + pCfB0262 | mat a his3 leu2 trp1 REV1p::BenM-KI.LEU2 SpHIS5 |
| | (Cen.PK113-5A) | |
| MeLS0153 | pCfB0262 + pMeLS0053 | mat a his3 leu2 trp1 RNR2p::BenM-KI.LEU2 SpHIS5 |
| | (Cen.PK113-5A) | |
| MeLS0079 | pMeLS0044 + pCfB0262 | mat a his3 leu2 trp1 TEF1p::BenM-KI.LEU2 SpHIS5 |
| | (Cen.PK113-5A) | |
| MeLS0152 | pCfB0262 + pMeLS0046 | mat a his3 leu2 trp1 TDH3p::BenM-KI.LEU2 SpHIS5 |
| | (Cen.PK113-5A) | |
| MeLS0131 | pMeLS0077 + pCfB257 | mat a his3 leu2 trp1 KI.LEU2 491bp_CYC1p::yEGFP-SpHIS5 |
| | (Cen.PK113-5A) | |
| MeLS0132 | pMeLS0078 + pCfB257 | mat a his3 leu2 trp1 KI.LEU2 272bp_CYC1p::yEGFP-SpHIS5 |
| | (Cen.PK113-5A) | |
| MeLS0133 | pMeLS0079 + pCfB257 | mat a his3 leu2 trp1 KI.LEU2 249bp_CYC1p::yEGFP-SpHIS5 |
| | (Cen.PK113-5A) | |
| MeLS0134 | pMeLS0080 + pCfB257 | mat a his3 leu2 trp1 KI.LEU2 209bp_CYC1p::yEGFP-SpHIS5 |
| | (Cen.PK113-5A) | |
| MeLS0135 | pMeLS0019 + pCfB257 | mat a his3 leu2 trp1 KI.LEU2 491bp_CYC1p_BenO_T1::yEGFP- |
| | (Cen.PK113-5A) | SpHIS5 |
| MeLS0136 | pMeLS0081 + pCfB257 | mat a his3 leu2 trp1 KI.LEU2 272bp_CYC1p_BenO_T1::yEGFP- |
| | (Cen.PK113-5A) | SpHIS5 |
| MeLS0137 | pMeLS0082 + pCfB257 | mat a his3 leu2 trp1 KI.LEU2 249bp_CYC1p_BenO_T1::yEGFP- |
| | (Cen.PK113-5A) | SpHIS5 |
| MeLS0138 | pMeLS0025 + pCfB257 | mat a his3 leu2 trp1 KI.LEU2 209bp_CYC1p_BenO_T1::yEGFP- |
| | (Cen.PK113-5A) | SpHIS5 |
| MeLS0177 | pMeLS0020 + pCfB257 | mat a his3 leu2 trp1 KI.LEU2 491bp_CYC1p_BenO_T2::yEGFP- |
| | (Cen.PK113-5A) | SpHIS5 |
| MeLS0259 | pMeLS0086 + pCfB257 | mat a his3 leu2 trp1 KI.LEU2 272bp_CYC1p_BenO_T2::yEGFP- |
| | (Cen.PK113-5A) | SpHIS5 |
| MeLS0260 | pMeLS0088 + pCfB257 | mat a his3 leu2 trp1 KI.LEU2 249bp_CYC1p_BenO_T2::yEGFP- |
| | (Cen.PK113-5A) | SpHIS5 |
| MeLS0180 | pMeLS0026 + pCfB257 | mat a his3 leu2 trp1 KI.LEU2 209bp_CYC1p_BenO_T2::yEGFP- |
| | (Cen.PK113-5A) | SpHIS5 |
| MeLS0178 | pMeLS0021 + pCfB257 | mat a his3 leu2 trp1 KI.LEU2 |
| | (Cen.PK113-5A) | 491bp_CYC1p_BenO_T1/T2::yEGFP-SpHIS5 |
| MeLS0261 | pMeLS0087 + pCfB257 | mat a his3 leu2 trp1 KI.LEU2 |
| | (Cen.PK113-5A) | 272bp_CYC1p_BenO_T1/T2::yEGFP-SpHIS5 |
| MeLS0262 | pMeLS0089 + pCfB257 | mat a his3 leu2 trp1 KI.LEU2 |
| | (Cen.PK113-5A) | 249bp_CYC1p_BenO_T1/T2::yEGFP-SpHIS5 |
| MeLS0181 | pMeLS0027 + pCfB257 | mat a his3 leu2 trp1 KI.LEU2 |
| | (Cen.PK113-5A) | 209bp_CYC1p_BenO_T1/T2::yEGFP-SpHIS5 |
| MeLS0164 | pMeLS0077 + pMeLS0046 | mat a his3 leu2 trp1 TDH3p::BenM-KI.LEU2 |
| | (Cen.PK113-5A) | 491bp_CYC1p::yEGFP-SpHIS5 |
| MeLS0165 | pMeLS0078 + pMeLS0046 | mat a his3 leu2 trp1 TDH3p::BenM-KI.LEU2 |
| | (Cen.PK113-5A) | 272bp_CYC1p::yEGFP-SpHIS5 |
| MeLS0166 | pMeLS0079 + pMeLS0046 | mat a his3 leu2 trp1 TDH3p::BenM-KI.LEU2 |
| | (Cen.PK113-5A) | 249bp_CYC1p::yEGFP-SpHIS5 |
| MeLS0167 | pMeLS0080 + pMeLS0046 | mat a his3 leu2 trp1 TDH3p::BenM-KI.LEU2 |
| | (Cen.PK113-5A) | 209bp_CYC1p::yEGFP-SpHIS5 |
| MeLS0156 | pMeLS0077 + pMeLS0044 | mat a his3 leu2 trp1 TEF1p::BenM-KI.LEU2 |
| | (Cen.PK113-5A) | 491bp_CYC1p::yEGFP-SpHIS5 |
| MeLS0157 | pMeLS0078 + pMeLS0044 | mat a his3 leu2 trp1 TEF1p::BenM-KI.LEU2 |
| | (Cen.PK113-5A) | 272bp_CYC1p::yEGFP-SpHIS5 |
| MeLS0158 | pMeLS0079 + pMeLS0044 | mat a his3 leu2 trp1 TEF1p::BenM-KI.LEU2 |
| | (Cen.PK113-5A) | 249bp_CYC1p::yEGFP-SpHIS5 |
| MeLS0159 | pMeLS0080 + pMeLS0044 | mat a his3 leu2 trp1 TEF1p::BenM-KI.LEU2 |
| | (Cen.PK113-5A) | 209bp_CYC1p::yEGFP-SpHIS5 |
| MeLS0160 | pMeLS0077 + pMeLS0053 | mat a his3 leu2 trp1 RNR2p::BenM-KI.LEU2 |
| | (Cen.PK113-5A) | 491bp_CYC1p::yEGFP-SpHIS5 |
| MeLS0161 | pMeLS0078 + pMeLS0053 | mat a his3 leu2 trp1 RNR2p::BenM-KI.LEU2 |
| | (Cen.PK113-5A) | 272bp_CYC1p::yEGFP-SpHIS5 |
| MeLS0162 | pMeLS0079 + pMeLS0053 | mat a his3 leu2 trp1 RNR2p::BenM-KI.LEU2 |
| | (Cen.PK113-5A) | 249bp_CYC1p::yEGFP-SpHIS5 |
| MeLS0163 | pMeLS0080 + pMeLS0053 | mat a his3 leu2 trp1 RNR2p::BenM-KI.LEU2 |
| | (Cen.PK113-5A) | 209bp_CYC1p::yEGFP-SpHIS5 |
| MeLS0139 | pMeLS0077 + pMeLS0045 | mat a his3 leu2 trp1 REV1p::BenM-KI.LEU2 |
| | (Cen.PK113-5A) | 491bp_CYC1p::yEGFP-SpHIS5 |
| MeLS0140 | pMeLS0078 + pMeLS0045 | mat a his3 leu2 trp1 REV1p::BenM-KI.LEU2 |
| | (Cen.PK113-5A) | 272bp_CYC1p::yEGFP-SpHIS5 |
| MeLS0141 | pMeLS0079 + pMeLS0045 | mat a his3 leu2 trp1 REV1p::BenM-KI.LEU2 |
| | (Cen.PK113-5A) | 249bp_CYC1p::yEGFP-SpHIS5 |
| MeLS0142 | pMeLS0080 + pMeLS0045 | mat a his3 leu2 trp1 REV1p::BenM-KI.LEU2 |
| | (Cen.PK113-5A) | 209bp_CYC1p::yEGFP-SpHIS5 |
| MeLS0172 | pMeLS0019 + pMeLS0046 | mat a his3 leu2 trp1 TDH3p::BenM-KI.LEU2 |
| | (Cen.PK113-5A) | 491bp_CYC1p_BenO_T1::yEGFP-SpHIS5 |
| MeLS0147 | pMeLS0081 + pMeLS0046 | mat a his3 leu2 trp1 TDH3p::BenM-KI.LEU2 |
| | (Cen.PK113-5A) | 272bp_CYC1p_BenO_T1::yEGFP-SpHIS5 |
| MeLS0173 | pMeLS0082 + pMeLS0046 | mat a his3 leu2 trp1 TDH3p::BenM-KI.LEU2 |
| | (Cen.PK113-5A) | 249bp_CYC1p_BenO_T1::yEGFP-SpHIS5 |
| MeLS0150 | pMeLS0025 + pMeLS0046 | mat a his3 leu2 trp1 TDH3p::BenM-KI.LEU2 |
| | (Cen.PK113-5A) | 209bp_CYC1p_BenO_T1::yEGFP-SpHIS5 |
| MeLS0168 | pMeLS0019 + pMeLS0044 | mat a his3 leu2 trp1 TEF1p::BenM-KI.LEU2 |
| | (Cen.PK113-5A) | 491bp_CYC1p_BenO_T1::yEGFP-SpHIS5 |
| MeLS0148 | pMeLS0081 + pMeLS0044 | mat a his3 leu2 trp1 TEF1p::BenM-KI.LEU2 |
| | (Cen.PK113-5A) | 272bp_CYC1p_BenO_T1::yEGFP-SpHIS5 |
| MeLS0169 | pMeLS0082 + pMeLS0044 | mat a his3 leu2 trp1 TEF1p::BenM-KI.LEU2 |
| | (Cen.PK113-5A) | 249bp_CYC1p_BenO_T1::yEGFP-SpHIS5 |
| MeLS0025 | pMeLS0025 + pMeLS0044 | mat a his3 leu2 trp1 TEF1p::BenM-KI.LEU2 |
| | (Cen.PK113-5A) | 209bp_CYC1p_BenO_T1::yEGFP-SpHIS5 |
| MeLS0170 | pMeLS0019 + pMeLS0053 | mat a his3 leu2 trp1 RNR2p::BenM-KI.LEU2 |
| | (Cen.PK113-5A) | 491bp_CYC1p_BenO_T1::yEGFP-SpHIS5 |
| MeLS0149 | pMeLS0081 + pMeLS0053 | mat a his3 leu2 trp1 RNR2p::BenM-KI.LEU2 |
| | (Cen.PK113-5A) | 272bp_CYC1p_BenO_T1::yEGFP-SpHIS5 |
| MeLS0171 | pMeLS0082 + pMeLS0053 | mat a his3 leu2 trp1 RNR2p::BenM-KI.LEU2 |
| | (Cen.PK113-5A) | 249bp_CYC1p_BenO_T1::yEGFP-SpHIS5 |
| MeLS0151 | pMeLS0025 + pMeLS0053 | mat a his3 leu2 trp1 RNR2p::BenM-KI.LEU2 |
| | (Cen.PK113-5A) | 209bp_CYC1p_BenO_T1::yEGFP-SpHIS5 |
| MeLS0143 | pMeLS0019 + pMeLS0045 | mat a his3 leu2 trp1 REV1p::BenM-KI.LEU2 |
| | (Cen.PK113-5A) | 491bp_CYC1p_BenO_T1::yEGFP-SpHIS5 |
| MeLS0144 | pMeLS0081 + pMeLS0045 | mat a his3 leu2 trp1 REV1p::BenM-KI.LEU2 |
| | (Cen.PK113-5A) | 272bp_CYC1p_BenO_T1::yEGFP-SpHIS5 |
| MeLS0145 | pMeLS0082 + pMeLS0045 | mat a his3 leu2 trp1 REV1p::BenM-KI.LEU2 |
| | (Cen.PK113-5A) | 249bp_CYC1p_BenO_T1::yEGFP-SpHIS5 |
| MeLS0049 | pMeLS0025 + pMeLS0045 | mat a his3 leu2 trp1 REV1p::BenM-KI.LEU2 |
| | (Cen.PK113-5A) | 209bp_CYC1p_BenO_T1::yEGFP-SpHIS5 |
| MeLS0263 | pMeLS0020 + pMeLS0046 | mat a his3 leu2 trp1 TDH3p::BenM-KI.LEU2 |
| | (Cen.PK113-5A) | 491bp_CYC1p_BenO_T2::yEGFP-SpHIS5 |
| MeLS0190 | pMeLS0086 + pMeLS0046 | mat a his3 leu2 trp1 TDH3p::BenM-KI.LEU2 |
| | (Cen.PK113-5A) | 272bp_CYC1p_BenO_T2::yEGFP-SpHIS5 |
| MeLS0192 | pMeLS0088 + pMeLS0046 | mat a his3 leu2 trp1 TDH3p::BenM-KI.LEU2 |
| | (Cen.PK113-5A) | 249bp_CYC1p_BenO_T2::yEGFP-SpHIS5 |
| MeLS0264 | pMeLS0026 + pMeLS0046 | mat a his3 leu2 trp1 TDH3p::BenM-KI.LEU2 |
| | (Cen.PK113-5A) | 209bp_CYC1p_BenO_T2::yEGFP-SpHIS5 |
| MeLS0020 | pMeLS0020 + pMeLS0044 | mat a his3 leu2 trp1 TEF1p::BenM-KI.LEU2 |
| | (Cen.PK113-5A) | 491bp_CYC1p_BenO_T2::yEGFP-SpHIS5 |
| MeLS0182 | pMeLS0086 + pMeLS0044 | mat a his3 leu2 trp1 TEF1p::BenM-KI.LEU2 |
| | (Cen.PK113-5A) | 272bp_CYC1p_BenO_T2::yEGFP-SpHIS5 |
| MeLS0184 | pMeLS0088 + pMeLS0044 | mat a his3 leu2 trp1 TEF1p::BenM-KI.LEU2 |
| | (Cen.PK113-5A) | 249bp_CYC1p_BenO_T2::yEGFP-SpHIS5 |
| MeLS0026 | pMeLS0026 + pMeLS0044 | mat a his3 leu2 trp1 TEF1p::BenM-KI.LEU2 |
| | (Cen.PK113-5A) | 209bp_CYC1p_BenO_T2::yEGFP-SpHIS5 |
| MeLS0265 | pMeLS0020 + pMeLS0053 | mat a his3 leu2 trp1 RNR2p::BenM-KI.LEU2 |
| | (Cen.PK113-5A) | 491bp_CYC1p_BenO_T2::yEGFP-SpHIS5 |
| MeLS0194 | pMeLS0086 + pMeLS0053 | mat a his3 leu2 trp1 RNR2p::BenM-KI.LEU2 |
| | (Cen.PK113-5A) | 272bp_CYC1p_BenO_T2::yEGFP-SpHIS5 |
| MeLS0196 | pMeLS0088 + pMeLS0053 | mat a his3 leu2 trp1 RNR2p::BenM-KI.LEU2 |
| | (Cen.PK113-5A) | 249bp_CYC1p_BenO_T2::yEGFP-SpHIS5 |
| MeLS0266 | pMeLS0026 + pMeLS0053 | mat a his3 leu2 trp1 RNR2p::BenM-KI.LEU2 |
| | (Cen.PK113-5A) | 209bp_CYC1p_BenO_T2::yEGFP-SpHIS5 |
| MeLS0044 | pMeLS0020 + pMeLS0045 | mat a his3 leu2 trp1 REV1p::BenM-KI.LEU2 |
| | (Cen.PK113-5A) | 491bp_CYC1p_BenO_T2::yEGFP-SpHIS5 |
| MeLS0186 | pMeLS0086 + pMeLS0045 | mat a his3 leu2 trp1 REV1p::BenM-KI.LEU2 |
| | (Cen.PK113-5A) | 272bp_CYC1p_BenO_T2::yEGFP-SpHIS5 |
| MeLS0188 | pMeLS0088 + pMeLS0045 | mat a his3 leu2 trp1 REV1p::BenM-KI.LEU2 |
| | (Cen.PK113-5A) | 249bp_CYC1p_BenO_T2::yEGFP-SpHIS5 |
| MeLS0050 | pMeLS0026 + pMeLS0045 | mat a his3 leu2 trp1 REV1p::BenM-KI.LEU2 |
| | (Cen.PK113-5A) | 209bp_CYC1p_BenO_T2::yEGFP-SpHIS5 |
| MeLS0267 | pMeLS0021 + pMeLS0046 | mat a his3 leu2 trp1 TDH3p::BenM-KI.LEU2 |
| | (Cen.PK113-5A) | 491bp_CYC1p_BenO_T1/2::yEGFP-SpHIS5 |
| MeLS0191 | pMeLS0087 + pMeLS0046 | mat a his3 leu2 trp1 TDH3p::BenM-KI.LEU2 |
| | (Cen.PK113-5A) | 272bp_CYC1p_BenO_T1/2::yEGFP-SpHIS5 |
| MeLS0193 | pMeLS0089 + pMeLS0046 | mat a his3 leu2 trp1 TDH3p::BenM-KI.LEU2 |
| | (Cen.PK113-5A) | 249bp_CYC1p_BenO_T1/2::yEGFP-SpHIS5 |
| MeLS0268 | pMeLS0027 + pMeLS0046 | mat a his3 leu2 trp1 TDH3p::BenM-KI.LEU2 |
| | (Cen.PK113-5A) | 209bp_CYC1p_BenO_T1/2::yEGFP-SpHIS5 |
| MeLS0021 | pMeLS0021 + pMeLS0044 | mat a his3 leu2 trp1 TEF1p::BenM-KI.LEU2 |
| | (Cen.PK113-5A) | 491bp_CYC1p_BenO_T1/2::yEGFP-SpHIS5 |
| MeLS0183 | pMeLS0087 + pMeLS0044 | mat a his3 leu2 trp1 TEF1p::BenM-KI.LEU2 |
| | (Cen.PK113-5A) | 272bp_CYC1p_BenO_T1/2::yEGFP-SpHIS5 |
| MeLS0185 | pMeLS0089 + pMeLS0044 | mat a his3 leu2 trp1 TEF1p::BenM-KI.LEU2 |
| | (Cen.PK113-5A) | 249bp_CYC1p_BenO_T1/2::yEGFP-SpHIS5 |
| MeLS0027 | pMeLS0027 + pMeLS0044 | mat a his3 leu2 trp1 TEF1p::BenM-KI.LEU2 |
| | (Cen.PK113-5A) | 209bp_CYC1p_BenO_T1/2::yEGFP-SpHIS5 |
| MeLS0269 | pMeLS0021 + pMeLS0053 | mat a his3 leu2 trp1 RNR2p::BenM-KI.LEU2 |
| | (Cen.PK113-5A) | 491bp_CYC1p_BenO_T1/2::yEGFP-SpHIS5 |
| MeLS0195 | pMeLS0087 + pMeLS0053 | mat a his3 leu2 trp1 RNR2p::BenM-KI.LEU2 |
| | (Cen.PK113-5A) | 272bp_CYC1p_BenO_T1/2::yEGFP-SpHIS5 |
| MeLS0197 | pMeLS0089 + pMeLS0053 | mat a his3 leu2 trp1 RNR2p::BenM-KI.LEU2 |
| | (Cen.PK113-5A) | 249bp_CYC1p_BenO_T/2::yEGFP-SpHIS5 |
| MeLS0270 | pMeLS0027 + pMeLS0053 | mat a his3 leu2 trp1 RNR2p::BenM-KI.LEU2 |
| | (Cen.PK113-5A) | 209bp_CYC1p_BenO_T1/2::yEGFP-SpHIS5 |
| MeLS0045 | pMeLS0021 + pMeLS0045 | mat a his3 leu2 trp1 REV1p::BenM-KI.LEU2 |
| | (Cen.PK113-5A) | 491bp_CYC1p_BenO_T1/2::yEGFP-SpHIS5 |
| MeLS0187 | pMeLS0087 + pMeLS0045 | mat a his3 leu2 trp1 REV1p::BenM-KI.LEU2 |
| | (Cen.PK113-5A) | 272bp_CYC1p_BenO_T1/2::yEGFP-SpHIS5 |
| MeLS0189 | pMeLS0089 + pMeLS0045 | mat a his3 leu2 trp1 REV1p::BenM-KI.LEU2 |
| | (Cen.PK113-5A) | 249bp_CYC1p_BenO_T1/2::yEGFP-SpHIS5 |
| MeLS0051 | pMeLS0027 + pMeLS0045 | mat a his3 leu2 trp1 REV1p::BenM-KI.LEU2 |
| | (Cen.PK113-5A) | 209bp_CYC1p_BenO_T1/2::yEGFP-SpHIS5 |
| MeLS0284 | pMeLS0025 + pMeLS0123 | mat a his3 leu2 trp1 REV1p::BenM(H110R, F211V, Y286N)- |
| | (Cen.PK113-5A) | KI.LEU2 209bp_CYC1p_BenO_T1::yEGFP-SpHIS5 |
| ST2377 | pCfB1237 + pCfB1239 | mat a ura3 his3 leu2 TDH3p::ScTkl1 TEF1p::KpAroY.D-SpHIS5 + |
| (CCM | (CEN.PK102-5B) | TDH3p::PaAroZ TEF1p::CaCatA-KILEU2 |
| intermediate; | | |
| with | | |
| ScTkl1) | | |
| ST3054 | pCfB1237 + pCfB2695 | mat a ura3 his3 leu2 TEF1p::KpAroY.D-SpHIS5 + |
| (CCM | (CEN.PK102-5B) | TDH3p::PaAroZ TEF1p::CaCatA-KILEU2 |
| intermediate; | | |
| no ScTkl1) | | |
| ST3034 | pCfB1241 (ST2377) | mat a ura3 his3 leu2 TDH3p::ScTkl1 TEF1p::KpAroY.D-SpHIS5 |
| (CCM- | | TDH3p::PaAroZ + TEF1p::CaCatA-KILEU2 + (TDH3p:: KpAroY.B |
| multiple; | | TEF1p::KpAroY.Ciso-KIURA3tag) × n |
| with ScTkl1) | | |
| ST3059 | pCfB2696 (ST2377) | mat a ura3 his3 leu2 TDH3p::ScTkl1 TEF1p::KpAroY.D-SpHIS5 + |
| (CCM-single; | | TDH3p::PaAroZ TEF1p::CaCatA-KILEU2 + TDH3p:: KpAroY.B |
| with ScTkl1) | | TEF1p::KpAroY.Ciso-KIURA3 |
| ST3058 | pCfB1241 (ST3054) | mat a ura3 his3 leu2 TEF1p::KpAroY.D-SpHIS5 + |
| (CCM- | | TDH3p::PaAroZ TEF1p::CaCatA-KILEU2 + (TDH3p:: KpAroY.B |
| multiple; no | | TEF1p::KpAroY.Ciso-KIURA3tag) × n |
| ScTkl1) | | |
| ST3154 | pCfB2696 (ST3054) | mat a ura3 his3 leu2 TEF1p::KpAroY.D-SpHIS5 + |
| (CCM-single; | | TDH3p::PaAroZ TEF1p::CaCatA-KILEU2 + TDH3p:: KpAroY.B |
| no ScTkl1) | | TEF1p::KpAroY.Ciso-KIURA3 |
| ST4240- | pCfB2553 + pCfB2764 | mat a URA3 HIS3 LEU2 TRP1 209bp_CYC1p_BenO_T1::yEGFP- |
| 1 (Reference | (CEN.PK113-7D) | HphMXsyn + REV1p::BenM(H110R, F211V, Y286N)-KanMXsyn |
| strain + | | |
| biosensor) | | |
| ST4241- | pCfB2553 + pCfB2764 | mat a ura3 his3 leu2 TEF1p::KpAroY.D-SpHIS5 + |
| 1 (CCM- | (ST3154) | TDH3p::PaAroZ TEF1p::CaCatA KILEU2 + TDH3p:: KpAroY.B |
| single; no | | TEF1p::KpAroY.Ciso-KIURA3 + 209bp_CYC1p_BenO_T1::yEGFP |
| ScTkl1 + | | HphMXsyn + REV1p::BenM(H110R, F211V, Y286N)-KanMXsyn |
| biosensor) | | |
| ST4242- | pCfB2553 + pCfB2764 | mat a ura3 his3 leu2 TDH3p::ScTkl1 TEF1p::KpAroY.D-SpHIS5 + |
| 8 (CCM- | (ST3059) | TDH3p::PaAroZ TEF1p::CaCatA-KILEU2 + TDH3p::KpAroY.B |
| single; with | | TEF1p::KpAroY.Ciso-KIURA3 + 209bp_CYC1p_BenO_T1::yEGFP |
| ScTkl1 + | | HphMXsyn + REV1p::BenM(H110R, F211V, Y286N)-KanMXsyn |
| biosensor) | | |
| ST4243- | pCfB2553 + pCfB2764 | mat a ura3 his3 leu2 TEF1p::KpAroY.D-SpHIS5 + |
| 1 (CCM- | (ST3058) | TDH3p::PaAroZ TEF1p::CaCatA-KILEU2 + (TDH3p::KpAroY.B |
| multiple; no | | TEF1p::KpAroY.Ciso-KIURA3tag) × n + |
| ScTkl1 + | | 209bp_CYC1p_BenO_T1::yEGFP HphMXsyn + |
| biosensor) | | REV1p::BenM(H110R, F211V, Y286N)-KanMXsyn |
| ST4244- | pCfB2553 + pCfB2764 | mat a ura3 his3 leu2 TDH3p::ScTkl1 TEF1p::KpAroY.D-SpHIS5 |
| 1 (CCM- | (ST3034) | TDH3p::PaAroZ + TEF1p::CaCatA-KILEU2 + (TDH3p:: KpAroY.B |
| multiple; | | TEF1p::KpAroY.Ciso-KIURA3tag) × n + |
| with ScTkl1 + | | 209bp_CYC1p_BenO_T1::yEGFP HphMXsyn + |
| biosensor) | | REV1p::BenM(H110R, F211V, Y286N)-KanMXsyn |
| ST4244- | pCfB2553 + pCfB2764 | mat a ura3 his3 leu2 TDH3p::ScTkl1 TEF1p::KpAroY.D-SpHIS5 |
| 2 (CCM- | (ST3034) | TDH3p::PaAroZ + TEF1p::CaCatA-KILEU2 + (TDH3p:: KpAroY.B |
| multiple; | | TEF1p::KpAroY.Ciso-KIURA3tag) × n + |
| with ScTkl1 + | | 209bp_CYC1p_BenO_T1::yEGFP HphMXsyn + |
| biosensor) | | REV1p::BenM(H110R, F211V, Y286N)-KanMXsyn |
| ST4245- | pCfB2553 + pCfB2764 | mat a ura3 his3 leu2 TDH3p::ScTkl1 TEF1p::KpAroY.D-SpHIS5 |
| 2 (CCM- | (ST3034) | TDH3p::PaAroZ + TEF1p::CaCatA-KILEU2 + (TDH3p:: KpAroY.B |
| multiple; | | TEF1p::KpAroY.Ciso-KIURA3tag) × n + |
| with ScTkl1 + | | 209bp_CYC1p_BenO_T1::yEGFP HphMXsyn + |
| biosensor) | | REV1p::BenM(H110R, F211V, Y286N)-KanMXsyn |
| ST4246- | pCfB2553 + pCfB2764 | mat a ura3 his3 leu2 TDH3p::ScTkl1 TEF1p::KpAroY.D-SpHIS5 |
| 2 (CCM- | (ST3034) | TDH3p::PaAroZ + TEF1p::CaCatA-KILEU2 + (TDH3p:: KpAroY.B |
| multiple; | | TEF1p::KpAroY.Ciso-KIURA3tag) × n + |
| with ScTkl1 + | | 209bp_CYC1p_BenO_T1::yEGFP HphMXsyn + |
| biosensor) | | REV1p::BenM(H110R, F211V, Y286N)-KanMXsyn |
| TISNO-64 | pTS-27 + pTS-21 | mat a his3 leu2 trp1 REV1p::PcaQ-KI.LEU2 |
| | (Cen.PK113-5A) | 209bp_CYC1p_PcaO_T1::yEGFP-SpHIS5 |
| TISNO-66 | pTS-29 + pTS-23 | mat a his3 leu2 trp1 REV1p::ArgP-KI.LEU2 |
| | (Cen.PK113-5A) | 209bp_CYC1p_ArgO_T1::yEGFP-SpHIS5 |
| TISNO-67 | pTS-30 + pTS-24 + pTS-39 | mat a his3 leu2 trp1 REV1p::MdcR-KI.LEU2 |
| | (Cen.PK113-5A) | 209bp_CYC1p_MdcO_T1::yEGFP-SpHIS5 TEF1pr::SpMAE1 |
| TISNO-71 | pTS-33 + pTS-21 | mat a his3 leu2 trp1 TDH3p::PcaQ-KI.LEU2 |
| | (Cen.PK113-5A) | 209bp_CYC1p_PcaO_T1::yEGFP-SpHIS5 |
| TISNO-73 | pTS-35 + pTS-23 | mat a his3 leu2 trp1 TDH3p::ArgP-KI.LEU2 |
| | (Cen.PK113-5A) | 209bp_CYC1p_ArgO_T1::yEGFP-SpHIS5 |
| TISNO-74 | pTS-36 + pTS-24 + pTS-39 | mat a his3 leu2 trp1 TDH3p::MdcR-KI.LEU2 |
| | (Cen.PK113-5A) | 209bp_CYC1p_MdcO_T1::yEGFP-SpHIS5 TEF1pr::SpMAE1 |
| TISNO-79 | pCfB257 + pTS-21 | mat a his3 leu2 trp1 KI.LEU2 209bp_CYC1p_PcaO_T1::yEGFP- |
| | (Cen.PK113-5A) | SpHIS5 |
| TISNO-81 | pCfB257 + pTS-23 | mat a his3 leu2 trp1 KI.LEU2 209bp_CYC1p_ArgO_T1::yEGFP- |
| | (Cen.PK113-5A) | SpHIS5 |
| TISNO-82 | pCfB257 + pTS-24 + pTS- | mat a his3 leu2 trp1 KI.LEU2 209bp_CYC1p_MdcO_T1::yEGFP- |
| | 39 (Cen.PK113-5A) | SpHIS5 TEF1pr::SpMAE1 |
| TISNO-83 | pCfB257 + pCfB2226 + | mat a his3 leu2 trp1 KI.LEU2 Sp.HIS5syn |
| | pTS-37 (Cen.PK113-5A) | 209bp_CYC1p_FdeO_T1::yEGFP-hphMXsyn |
| TISNO-89 | pTS-36 + pTS-23 | mat a his3 leu2 trp1 TDH3p::MdcR-KI.LEU2 |
| | (Cen.PK113-5A) | 209bp_CYC1p_ArgO_T1::yEGFP-SpHIS5 |
| TISNO-90 | pTS-35 + pTS-24 + pTS-39 | mat a his3 leu2 trp1 TDH3p::ArgP-KI.LEU2 |
| | (Cen.PK113-5A) | 209bp_CYC1p_MdcO_T1::yEGFP-SpHIS5 TEF1pr::SpMAE1 |
| TISNO-93 | pTS-45 + pTS-37 | mat a ura3 his3 leu2 TDH3p::FdeR-KI.URA3syn |
| | (Cen.PK102-5B) | 209bp_CYC1p_FdeO_T1::yEGFP-hphMXsyn |
| TISNO-95 | pMije0124 + pTS-37 | mat a ura3 his3 leu2 REV1p::FdeR-KI.LEU2 |
| | (Cen.PK102-56) | 209bp_CYC1p_FdeO_T1::yEGFP-hphMXsyn |
| EV0 | — | mat a ura3::LoxP-KanMX-LoxP pad1-fdc1::LoxP-NATMX-LoxP |
| | | aro10Δ0 |
| EV1 | pROP280 + pROP266 + | mat a ura3::LoxP-KanMX-LoxP pad1-fdc1::LoxP-NATMX-LoxP |
| | pROP273 (EV0) | aro10Δ0 + TDH3p::AtPAL2 TEF2p::C4H::L5::ATR2 |
| | | PGK1p::HaCHS TEF1p::PhCHI PDC1p::At4Cl |
| EV2 | pROP338 + pROP339 + | mat a ura3::LoxP-KanMX-LoxP pad1-fdc1::LoxP-NATMX-LoxP |
| | pROP191 (EV1) | aro10Δ0 + TDH3p::AtPAL2 TEF2p::C4H::L5::ATR2 |
| | | PGK1p::HaCHS TEF1p::PhCHI PDC1p::At4Cl + TDH3p::AtPAL2 |
| | | PGK1p::HaCHS |
| EV3 | pROP423 + pROP339 + | mat a ura3::LoxP-KanMX-LoxP pad1-fdc1::LoxP-NATMX-LoxP |
| | pVAN968 (EV2) | aro10Δ0 + TDH3p::AtPAL2 TEF2p::C4H::L5::ATR2 |
| | | PGK1p::HaCHS TEF1p::PhCHI PDC1p::At4Cl + TDH3p::AtPAL2 |
| | | PGK1p::HaCHS + TDH3p::AtPAL2 TEF2p::C4H::L5::ATR2 |
| | | PGK1p::HaCHS |
| EVR0 (ctrl) | pCfB2198 + pTS-49 (EV0) | mat a ura3::LoxP-KanMX-LoxP pad1-fdc1::LoxP-NATMX-LoxP |
| | | aro10Δ0 + 209bp_CYC1p_FdeO_T1::yEGFP-hphMXsyn1 + |
| | | [CEN/ARS/URA3/TDH3p::FdeR] |
| EVR1 | pCfB2198 + pTS-49 (EV1) | mat a ura3::LoxP-KanMX-LoxP pad1-fdc1::LoxP-NATMX-LoxP |
| | | aro10Δ0 + TDH3p::AtPAL2 TEF2p::C4H::L5::ATR2 |
| | | PGK1p::HaCHS TEF1p::PhCHI PDC1p::At4Cl + |
| | | 209bp_CYC1p_FdeO_T1::yEGFP-hphMXsyn1 + |
| | | [CEN/ARS/URA3/TDH3p::FdeR] |
| EVR2 | pCfB2198 + pTS-49 (EV2) | mat a ura3::LoxP-KanMX-LoxP pad1-fdc1::LoxP-NATMX-LoxP |
| | | aro10Δ0 + TDH3p::AtPAL2 TEF2p::C4H::L5::ATR2 |
| | | PGK1p::HaCHS TEF1p::PhCHI PDC1p::At4Cl + TDH3p::AtPAL2 |
| | | PGK1p::HaCHS + 209bp_CYC1p_FdeO_T1::yEGFP-hphMXsyn1 + |
| | | [CEN/ARS/URA3/TDH3p::FdeR] |
| EVR3 | pCfB2198 + pTS-49 (EV3) | mat a ura3::LoxP-KanMX-LoxP pad1-fdc1::LoxP-NATMX-LoxP |
| | | aro10Δ0 + TDH3p::AtPAL2 TEF2p::C4H::L5::ATR2 |
| | | PGK1p::HaCHS TEF1p::PhCHI PDC1p::At4Cl + TDH3p::AtPAL2 |
| | | PGK1p::HaCHS + TDH3p::AtPAL2 TEF2p::C4H::L5::ATR2 |
| | | PGK1p::HaCHS + 209bp_CYC1p_FdeO_T1::yEGFP-hphMXsyn1 + |
| | | [CEN/ARS/URA3/TDH3p::FdeR] |

TABLE 2
Mean fluorescense intensities of GFP (24 h)

| | | | CCM | sd | sd | FC |
|---|---|---|---|---|---|---|
| Strain ID | Design | Ctrl | (1.4 mM) | ctrl | CCM | (+/−CCM) |
| MeLS0081 | REV1p-BenM | 159 | 175 | 3 | 7 | 1.105042017 |
| MeLS0153 | RNR2p-BenM | 154 | 174 | 2 | 2 | 1.130151844 |
| MeLS0079 | TEF1p-BenM | 173 | 185 | 4 | 12 | 1.071428571 |
| MeLS0152 | TDH3p-BenM | 170 | 184 | 12 | 3 | 1.080392157 |
| MeLS0136 | 272bp_CYC1p_BenO_T1:GFP | 155 | 160 | 0 | 1 | 1.032258065 |
| MeLS0162 | RNR2p-BenM + 249bp_CYC1p:GFP | 146 | 164 | 2 | 4 | 1.120728929 |
| MeLS0163 | RNR2p-BenM + 209bp_CYC1p:GFP | 146 | 164 | 4 | 3 | 1.120728929 |
| MeLS0138 | 209bp_CYC1p_BenO_T1:GFP | 147 | 167 | 5 | 4 | 1.133484163 |
| MeLS0189 | REV1p-BenM + | 148 | 181 | 3 | 8 | 1.220224719 |
| | 249bp_CYC1p_BenO_T1/T2:GFP | | | | | |
| MeLS0181 | 209bp_CYC1p_BenO_T1/T2:GFP | 150 | 165 | 7 | 6 | 1.102222222 |
| MeLS0134 | 209bp_CYC1p:GFP | 151 | 171 | 4 | 4 | 1.129955947 |
| MeLS0180 | 209bp_CYC1p_BenO_T2:GFP | 152 | 165 | 3 | 5 | 1.087719298 |
| MeLS0159 | TEF1p-BenM + 209bp_CYC1p:GFP | 156 | 177 | 6 | 7 | 1.132478632 |
| MeLS0137 | 249bp_CYC1p_BenO_T1:GFP | 156 | 180 | 1 | 1 | 1.151385928 |
| MeLS0050 | REV1p-BenM + | 156 | 182 | 6 | 4 | 1.166311301 |
| | 209bp_CYC1p_BenO_T2:GFP | | | | | |
| MeLS0158 | TEF1p-BenM + 249bp_CYC1p:GFP | 157 | 175 | 1 | 2 | 1.116772824 |
| MeLS0142 | REV1p-BenM + 209bp_CYC1p:GFP | 159 | 174 | 1 | 2 | 1.089958159 |
| MeLS0188 | REV1p-BenM + | 160 | 194 | 5 | 5 | 1.214583333 |
| | 249bp_CYC1p_BenO_T2:GFP | | | | | |
| MeLS0195 | RNR2p-BenM + | 161 | 199 | 6 | 7 | 1.238095238 |
| | 272bp_CYC1p_BenO_T1/T2:GFP | | | | | |
| MeLS0197 | RNR2p-BenM + | 163 | 256 | 2 | 15 | 1.570552147 |
| | 249bp_CYC1p_BenO_T1/T2:GFP | | | | | |
| MeLS0186 | REV1p-BenM + | 164 | 197 | 5 | 3 | 1.201219512 |
| | 272bp_CYC1p_BenO_T2:GFP | | | | | |
| MeLS0140 | REV1p-BenM + 272bp_CYC1p:GFP | 166 | 183 | 5 | 3 | 1.104627767 |
| MeLS0141 | REV1p-BenM + 249bp_CYC1p:GFP | 167 | 176 | 8 | 3 | 1.055888224 |
| MeLS0266 | RNR2p-BenM + | 167 | 179 | 5 | 3 | 1.071856287 |
| | 209bp_CYC1p_BenO_T2:GFP | | | | | |
| MeLS0161 | RNR2p-BenM + 272bp_CYC1p:GFP | 171 | 180 | 11 | 8 | 1.048638132 |
| MeLS0132 | 272bp_CYC1p:GFP | 174 | 211 | 6 | 7 | 1.213051823 |
| MeLS0157 | TEF1p-BenM + 272bp_CYC1p:GFP | 175 | 198 | 8 | 26 | 1.12952381 |
| MeLS0167 | TDH3p-BenM + 209bp_CYC1p:GFP | 178 | 194 | 4 | 12 | 1.090056285 |
| MeLS0166 | TDH3p-BenM + 249bp_CYC1p:GFP | 181 | 205 | 1 | 10 | 1.130514706 |
| MeLS0182 | TEF1p-BenM + | 183 | 211 | 5 | 11 | 1.155109489 |
| | 272bp_CYC1p_BenO_T2:GFP | | | | | |
| MeLS0165 | TDH3p-BenM + 272bp_CYC1p:GFP | 187 | 216 | 2 | 21 | 1.156862745 |
| MeLS0171 | RNR2p-BenM + | 187 | 290 | 6 | 4 | 1.549019608 |
| | 249bp_CYC1p_BenO_T1:GFP | | | | | |
| MeLS0051 | REV1p-BenM + | 196 | 307 | 5 | 3 | 1.568994889 |
| | 209bp_CYC1p_BenO_T1/T2:GFP | | | | | |
| MeLS0070 | RNR2p-BenM + | 200 | 356 | 3 | 14 | 1.775374376 |
| | 209bp_CYC1p_BenO_T1/T2:GFP | | | | | |
| MeLS0190 | TDH3p-BenM + | 201 | 218 | 7 | 7 | 1.088039867 |
| | 272bp_CYC1p_BenO_T2:GFP | | | | | |
| MeLS0259 | 272bp_CYC1p_BenO_T2:GFP | 201 | 201 | 4 | 12 | 1.001658375 |
| MeLS0262 | 249bp_CYC1p_BenO_T1/T2:GFP | 203 | 203 | 7 | 5 | 1 |
| MeLS0187 | REV1p-BenM + | 207 | 370 | 7 | 4 | 1.789049919 |
| | 272bp_CYC1p_BenO_T1/T2:GFP | | | | | |
| MeLS0145 | REV1p-BenM + | 207 | 405 | 2 | 17 | 1.953376206 |
| | 249bp_CYC1p_BenO_T1:GFP | | | | | |
| MeLS0133 | 249bp_CYC1p:GFP | 210 | 219 | 11 | 20 | 1.042789223 |
| MeLS0196 | RNR2p-BenM + | 221 | 349 | 7 | 10 | 1.575301205 |
| | 249bp_CYC1p_BenO_T2:GFP | | | | | |
| MeLS0151 | RNR2p-BenM + | 230 | 859 | 6 | 23 | 3.734782609 |
| | 209bp_CYC1p_BenO_T1:GFP | | | | | |
| MeLS0049 | REV1p-BenM + | 231 | 880 | 37 | 6 | 3.80952381 |
| | 209bp_CYC1p_BenO_T1:GFP | | | | | |
| MelS0194 | RNR2p-BenM + | 242 | 258 | 9 | 13 | 1.064649243 |
| | 272bp_CYC1p_BenO_T2:GFP | | | | | |
| MeLS0178 | 491bp_CYC1p_BenO_T1/T2:GFP | 248 | 241 | 4 | 3 | 0.97311828 |
| MeLS0183 | TEF1p-BenM + | 259 | 511 | 4 | 11 | 1.969151671 |
| | 272bp_CYC1p_BenO_T1/T2:GFP | | | | | |
| MeLS0184 | TEF1p-BenM + | 287 | 475 | 2 | 8 | 1.653132251 |
| | 249bp_CYC1p_BenO_T2:GFP | | | | | |
| MeLS0261 | 272bp_CYC1p_BenO_T1/T2:GFP | 321 | 257 | 17 | 2 | 0.798755187 |
| MeLS0144 | REV1p-BenM + | 338 | 804 | 4 | 23 | 2.381046397 |
| | 272bp_CYC1p_BenO_T1:GFP | | | | | |
| MeLS0260 | 249bp_CYC1p_BenO_T2:GFP | 344 | 313 | 11 | 20 | 0.909002904 |
| MeLS0264 | TDH3p-BenM + | 353 | 527 | 7 | 5 | 1.495274102 |
| | 209bp_CYC1p_BenO_T2:GFP | | | | | |
| MeLS0020 | TEF1p-BenM + | 365 | 813 | 14 | 18 | 2.224452555 |
| | 491bp_CYC1p_BenO_T2:GFP | | | | | |
| MeLS0191 | TDH3p-BenM + | 365 | 691 | 12 | 43 | 1.892335766 |
| | 272bp_CYC1p_BenO_T1/T2:GFP | | | | | |
| MeLS0149 | RNR2p-BenM + | 369 | 854 | 21 | 15 | 2.313459801 |
| | 272bp_CYC1p_BenO_T1:GFP | | | | | |
| MeLS0026 | TEF1p-BenM + | 380 | 406 | 5 | 8 | 1.068481124 |
| | 209bp_CYC1p_BenO_T2:GFP | | | | | |
| MeLS0193 | TDH3p-BenM + | 407 | 1630 | 10 | 67 | 4.004095004 |
| | 249bp_CYC1p_BenO_T1/T2:GFP | | | | | |
| MeLS0044 | REV1p-BenM + | 438 | 667 | 18 | 7 | 1.523990861 |
| | 491bp_CYC1p_BenO_T2:GFP | | | | | |
| MeLS0265 | RNR2p-BenM + | 461 | 698 | 22 | 21 | 1.51300578 |
| | 491bp_CYC1p_BenO_T2:GFP | | | | | |
| MeLS0027 | TEF1p-BenM + | 554 | 1879 | 60 | 20 | 3.393738712 |
| | 209bp_CYC1p_BenO_T1/T2:GFP | | | | | |
| MeLS0192 | TDH3p-BenM + | 555 | 978 | 13 | 22 | 1.760504202 |
| | 249bp_CYC1p_BenO_T2:GFP | | | | | |
| MeLS0263 | TDH3p-BenM + | 631 | 1197 | 15 | 15 | 1.897517169 |
| | 491bp_CYC1p_BenO_T2:GFP | | | | | |
| MeLS0177 | 491bp_CYC1p_BenO_T2:GFP | 641 | 698 | 18 | 7 | 1.088403536 |
| MeLS0268 | TDH3p-BenM + | 668 | 2172 | 14 | 96 | 3.250374065 |
| | 209bp_CYC1p_BenO_T1/T2:GFP | | | | | |
| MeLS0169 | TEF1p-BenM + | 672 | 2913 | 12 | 21 | 4.337468983 |
| | 249bp_CYC1p_BenO_T1:GFP | | | | | |
| MeLS0135 | 491bp_CYC1p_BenO_T1:GFP | 754 | 801 | 12 | 11 | 1.061864781 |
| MeLS0173 | TDH3p-BenM + | 755 | 3554 | 6 | 78 | 4.706843267 |
| | 249bp_CYC1p_BenO_T1:GFP | | | | | |
| MeLS0267 | TDH3p-BenM + | 840 | 2218 | 33 | 81 | 2.639825466 |
| | 491bp_CYC1p_BenO_T1/T2:GFP | | | | | |
| MeLS0148 | TEF1p-BenM + | 1164 | 4032 | 39 | 145 | 3.463212139 |
| | 272bp_CYC1p_BenO_T1:GFP | | | | | |
| MeLS0025 | TEF1p-BenM + | 1185 | 4139 | 6 | 85 | 3.493528419 |
| | 209bp_CYC1p_BenO_T1:GFP | | | | | |
| MeLS0150 | TDH3p-BenM + | 1192 | 4868 | 16 | 47 | 4.082471345 |
| | 209bp_CYC1p_BenO_T1:GFP | | | | | |
| MeLS0045 | REV1p-BenM + | 1197 | 1698 | 8 | 67 | 1.418151448 |
| | 491bp_CYC1p_BenO_T1/T2:GFP | | | | | |
| MeLS0269 | RNR2p-BenM + | 1291 | 2589 | 80 | 74 | 2.005422153 |
| | 491bp_CYC1p_BenO_T1/T2:GFP | | | | | |
| MeLS0147 | TDH3p-BenM + | 1333 | 4984 | 28 | 65 | 3.738 |
| | 272bp_CYC1p_BenO_T1:GFP | | | | | |
| MeLS0021 | TEF1p-BenM + | 1358 | 2967 | 16 | 142 | 2.18404908 |
| | 491bp_CYC1p_BenO_T1/T2:GFP | | | | | |
| MeLS0139 | REV1p-BenM + 491bp_CYC1p:GFP | 2270 | 2231 | 1813 | 1771 | 0.98281686 |
| MeLS0160 | RNR2p-BenM + 491bp_2YC1p:GFP | 2625 | 2811 | 39 | 118 | 1.07059421 |
| MeLS0185 | TEF1p-BenM + | 2632 | 9992 | 273 | 294 | 3.796833439 |
| | 249bp_CYC1p_BenO_T1/T2:GFP | | | | | |
| MeLS0131 | 491bp_CYC1p:GFP | 2781 | 2899 | 30 | 116 | 1.042435867 |
| MeLS0156 | TEF1p-BenM + 491bp_CYC1p:GFP | 3046 | 3016 | 44 | 67 | 0.990152095 |
| MeLS0164 | TDH3p-BenM + 491bp_CYC1p:GFP | 4003 | 4509 | 90 | 105 | 1.126488467 |
| MeLS0143 | REV1p-BenM + | 5321 | 5789 | 181 | 229 | 1.0880842 |
| | 491bp_CYC1p_BenO_T1:GFP | | | | | |
| MeLS0170 | RNR2p-BenM + | 5616 | 6114 | 87 | 48 | 1.088734568 |
| | 491bp_CYC1p_BenO_T1:GFP | | | | | |
| MeLS0172 | TDH3p-BenM + | 6894 | 10605 | 24 | 353 | 1.53834252 |
| | 491bp_CYC1p_BenO_T1:GFP | | | | | |
| MeLS0168 | TEF1p-BenM + | 15429 | 20250 | 355 | 316 | 1.312441938 |
| | 491bp_CYC1p_BenO_T1:GFP | | | | | |

TABLE 3
List of plasmids

| Plasmid | Parent | | |
|---|---|---|---|
| name | plasmid | Description | Reference/Source |
| pCfB258 | — | pX-4-LoxP-SpHIS5 | Jensen et al., 2014 |
| pCfB322 | — | pTY4-LoxP-KIURA3tag | Borodina et al., 2014 |
| pCfB388 | — | pXI-1-LoxP-KILEU2 | Jensen et al., 2014 |
| pCfB390 | — | pXI-3-LoxP-KIURA3 | Jensen et al., 2014 |
| pCfB2198 | — | pXII-4-LoxP-HphMXsyn | Stovicek et al., 2015 |
| pCfB2223 | — | pX-3-LoxP-KanMXsyn | Stovicek et al., 2015 |
| pCfB2226 | — | pX-4-IoxP-SpHIS5syn | Stovicek et al., 2015 |
| pCf61237 | pCfB258 | pX-4-IoxP-SpHiS5-ScTkl1<-TDH3p-TEF1p-> | This study |
| | | KpAroY.D | |
| pCfB1239 | pCfB388 | pXI-1-LoxP-KILEU2-PaAroZ<-TDH3p-TEF1p-> | This study |
| | | CaCatA | |
| pCfB1241 | pCfB322 | pTY4-LoxP-KIURA3tag-KpAroY.B<-TDH3p- | This study |
| | | TEF1p->KpAroY.Ciso | |
| pCfB2374 | — | pXI-1-IoxP-KIURA3syn | Stovicek et al., 2015 |
| pCfB3039 | — | pXII-2 | Stovicek et al., 2015 |
| pCfB2695 | pCfB258 | pX-4-LoxP-SpHiS5-TEF1p->KpAroY.D | This study |
| pCfB2696 | pCfB390 | pXI-3-KIURA3-KpAroY.6<-TDH3p-TEF1p-> | This study |
| | | KpAroY.Ciso | |
| pCfB2553 | pCfB2198 | pXII-4-LoxP-HphMXsyn- | This study |
| | | 209bp_CYC1p_BenO_T1->yEGFP | |
| pCfB2764 | pCfB2223 | pX-3-LoxP-KanMXsyn-REV1p->BenM(H110R, | This study |
| | | F211V, Y286N) | |
| pCfB257 | — | pX-3-LoxP-KILEU2 | Jensen et al., 2014 |
| pCfB262 | — | pXII-4-LoxP-SpHIS5 | Jensen et al., 2014 |
| pRS416U | — | URA3, USER cassette | This study |
| pMeLS0045 | pCfB257 | pX-3-LoxP-KILEU2-REV1p->BenM | This study |
| pMeLS0044 | pCfB257 | pX-3-LoxP-KILEU2-TEF1p->BenM | This study |
| pMeLS0053 | pCfB257 | pX-3-LoxP-KILEU2-RNR2p->BenM | This study |
| pMeLS0046 | pCfB257 | pX-3-LoxP-KILEU2-TDH3p->BenM | This study |
| pMeLS0077 | pCfB262 | pXII-4-LoxP-SpHIS5-491bp_CYC1p->yeGFP | This study |
| pMeLS0078 | pCfB262 | pXII-4-LoxP-SpHIS5-272bp_CYC1p->yeGFP | This study |
| pMeLS0079 | pCfB262 | pXII-4-LoxP-SpHIS5-249bp_CYC1p->yeGFP | This study |
| pMeLS0080 | pCfB262 | pXII-4-LoxP-SpHIS5-209bp_CYC1p->yeGFP | This study |
| pMeLS0019 | pCfB262 | pXII-4-LoxP-SpHIS5-491bp_CYC1p_BenO_T1-> | This study |
| | | yeGFP | |
| pMeLS0081 | pCfB262 | pXII-4-LoxP-SpHIS5-272bp_CYC1p_BenO_T1-> | This study |
| | | yeGFP | |
| pMeLS0082 | pCfB262 | pXII-4-LoxP-SpHIS5-249bp_CYC1p_BenO_T1-> | This study |
| | | yeGFP | |
| pMeLS0025 | pCfB262 | pXII-4-LoxP-SpHIS5-209bp_CYC1p_BenO_T1-> | This study |
| | | yeGFP | |
| pMeLS0020 | pCfB262 | pXII-4-LoxP-SpHIS5-491bp_CYC1p_BenO_T2-> | This study |
| | | yeGFP | |
| pMeLS0086 | pCfB262 | pXII-4-LoxP-SpHIS5-272bp_CYC1p_BenO_T2-> | This study |
| | | yeGFP | |
| pMeLS0088 | pCfB262 | pXII-4-LoxP-SpHIS5-249bp_CYC1p_BenO_T2-> | This study |
| | | yeGFP | |
| pMeLS0026 | pCfB262 | pXII-4-LoxP-SpHIS5-209bp_CYC1p_BenO_T2-> | This study |
| | | yeGFP | |
| pMeLS0021 | pCfB262 | pXII-4-LoxP-SpHIS5- | This study |
| | | 491bp_CYC1p_BenO_T1/2->yeGFP | |
| pMeLS0087 | pCfB262 | pXII-4-LoxP-SpHIS5- | This study |
| | | 272bp_CYC1p_BenO_T1/2->yeGFP | |
| pMeLS0089 | pCfB262 | pXII-4-LoxP-SpHIS5- | This study |
| | | 249bp_CYC1p_BenO_T1/2->yeGFP | |
| pMeLS0027 | pCfB262 | pXII-4-LoxP-SpHIS5- | This study |
| | | 209bp_CYC1p_BenO_T1/2->yeGFP | |
| pMeLS0123 | pCfB257 | pX-3-LoxP-KILEU2-REV1p->BenM(H110R, | This study |
| | | F211V, Y286N) | |
| pMije0124 | pCfB257 | pX-3-LoxP-KILEU2-REV1p->FdeR | This study |
| pTS-21 | pCfB262 | pXII-4-LoxP-SpHIS5-209bp_CYC1p_PcaO_T1-> | This study |
| | | yeGFP | |
| pTS-23 | pCfB262 | pXII-4-LoxP-SpHIS5-209bp_CYC1p_ArgO_T1-> | This study |
| | | yeGFP | |
| pTS-24 | pCfB262 | pXII-4-LoxP-SpHIS5-209bp_CYC1p_MdcO_T1-> | This study |
| | | yeGFP | |
| pTS-27 | pCfB257 | pX-3-LoxP-KILEU2-REV1p->PcaQ | This study |
| pTS-29 | pCfB257 | pX-3-LoxP-KILEU2-REV1p->ArgP | This study |
| pTS-30 | pCfB257 | pX-3-LoxP-KILEU2-REV1p->MdcR | This study |
| pTS-33 | pCfB257 | pX-3-LoxP-KILEU2-TDH3p->PcaQ | This study |
| pTS-35 | pCfB257 | pX-3-LoxP-KILEU2-TDH3p->ArgP | This study |
| pTS-36 | pCfB257 | pX-3-LoxP-KILEU2-TDH3p->MdcR | This study |
| pTS-37 | pCfB2198 | pXII-4-LoxP-HphMX-209bp_CYC1p_FdeO_T1-> | This study |
| | | yeGFP | |
| pTS-38 | pCfB2374 | pXI-1-LoxP-KI.URA3syn-TDH3p->FdeR | This study |
| pTS-39 | pCfB3039 | pXII-2-TEF1p->Sp.MAE1 | This study |
| pTS-49 | pRS416U | pURA3-TDH3p->FdeR | This study |
| pROP280 | — | pX-DR-KILEU2-AtPAL2<-TDH3p-TEF2p-> | This study |
| | | AtC4H::L5::AtATR2 | |
| pROP266 | — | HaCHS<-PGK1p-TEF1p->PhCHI | This study |
| pROP273 | — | pX-PDC1p->At4CL2 | This study |
| pROP338 | — | pXI-DR-KILEU2-AtPAL2<-TDH3p | This study |
| pROP339 | — | HaCHS<-PGK1p | This study |
| pROP191 | — | pXI | This study |
| pROP423 | — | pXVI-DR-KILEU2-AtPAL2<-TDH3p-TEF2p-> | This study |
| | | AtC4H::L5::AtATR2 | |
| pVAN968 | — | pXVI | This study |

TABLE 4
List of synthetic genes. For synthetic reporter promoters based on the
209 bp_CYC1p scaffold promoter, LTTR operator sites are marked in bold and start
codon for yeGFP is marked in green.

LOCUS CaCatA 912 bp

| | |
|---|---|
| 1 | ATGTCCCAAG CTTTCACCGA ATCTGTTAAG ACTTCTTTGG GTCCAAATGC TACTCCAAGA |
| 61 | GCTAAAAAGT TGATTGCCTC TTTGGTTCAA CACGTTCATG ATTTCGCTAG AGAAAACCAT |
| 121 | TTGACTACCG AAGATTGGTT GTGGGGTGTT GATTTCATTA ACAGAATTGG TCAAATGTCC |
| 181 | GACTCCAGAA GAAACGAAGG TATTTTGGTT TGCGATATCA TCGGTTTGGA AACCTTGGTT |
| 241 | GATGCTTTGA CTAACGAATC CGAACAATCT AACCATACCT CCTCTGCTAT TTTGGGTCCT |
| 301 | TTTTACTTGC CAGATTCTCC AGTTTATCCA AACGGTGGTT CTATCGTTCA AAAGGCTATT |
| 361 | CCAACTGATG TTAAGTGCTT CGTTAGAGGT AAGGTTACTG ATACTGAAGG TAAACCATTG |
| 421 | GGTGGTGCTC AATTGGAAGT TTGGCAATGT AATTCTGCTG GTTTCTACTC TCAACAAGCT |
| 481 | GATCATGATG GTCCAGAATT CAATTTGAGA GGTACTTTCA TTACCGACGA CGAAGGTAAT |
| 541 | TACTCCTTCG AATGTTTAAG ACCAACCTCC TATCCAATTC CATACGATGG TCCTGCTGGT |
| 601 | GATTTGTTGA AAATCATGGA TAGACATCCA AACAGACCAT CCCATATTCA TTGGAGAGTT |
| 661 | TCTCATCCAG GTTACCATAC TTTGATCACC CAAATCTATG ATGCTGAATG TCCATACACC |
| 721 | AACAACGATT CTGTTTACGC TGTTAAGGAT GACATCATCG TTCACTTCGA AAAGGTTGAT |
| 781 | AACAAGGATA AGGATTTGGT CGGTAAGGTC GAATACAAGT TGGATTACGA TATTTCCTTG |
| 841 | GCCACCGAAT CCTCTATTCA AGAAGCTAGA GCTGCTGCTA AAGCTAGACA AGATGCTGAA |
| 901 | ATCAAGTTGT AA |
| // | |

LOCUS KpAroY.B 594 bp

| | |
|---|---|
| 1 | ATGAAGTTGA TCATCGGTAT GACTGGTGCT ACAGGTGCTC CATTGGGTGT TGCTTTGTTG |
| 61 | CAAGCTTTGA GAGATATGCC AGAAGTTGAA ACCCATTTGG TTATGTCTAA ATGGGCTAAG |
| 121 | ACCACCATTG AATTGGAAAC TCCATGGACT GCTAGAGAAG TTGCTGCTTT GGCTGATTTT |
| 181 | TCTCATTCTC CAGCTGATCA AGCTGCTACT ATTTCTTCTG GTTCTTTCAG AACTGATGGT |
| 241 | ATGATCGTTA TTCCATGCTC TATGAAAACC TTGGCTGGTA TTAGAGCTGG TTATGCTGAA |
| 301 | GGTTTGGTTG GTAGAGCTGC TGATGTTGTT TTGAAAGAAG GTAGAAAGTT GGTCTTGGTC |
| 361 | CCAAGAGAAA TGCCATTGTC TACTATCCAT TTGGAAAACA TGTTGGCCTT GTCTAGAATG |
| 421 | GGTGTAGCTA TGGTTCCACC AATGCCAGCT TATTACAATC ATCCAGAAAC CGTTGATGAC |
| 481 | ATCACCAACC ATATAGTTAC CAGAGTTTTG GACCAATTCG GTTTGGATTA TCACAAAGCT |
| 541 | AGAAGATGGA ACGGTTTGAG AACTGCTGAA CAATTCGCTC AAGAAATTGA ATGA |
| // | |

LOCUS KpAroY.Ciso 1509 bp

| | |
|---|---|
| 1 | ATGACCGCCC CAATCCAAGA TTTGAGAGAT GCTATTGCTT TGTTACAACA ACACGACAAT |
| 61 | CAATACTTGG AAACCGATCA TCCAGTTGAT CCAAATGCTG AATTGGCTGG TGTTTACAGA |
| 121 | CATATTGGTG CTGGTGGTAC TGTAAAAAGA CCAACTAGAA TTGGTCCAGC CATGATGTTC |
| 181 | AACAACATTA AGGGTTATCC ACACTCCAGA ATCTTGGTTG GTATGCATGC TTCTAGACAA |
| 241 | AGAGCAGCTT TGTTGTTGGG TTGTGAAGCT TCTCAATTGG CTTTGGAAGT TGGTAAAGCT |
| 301 | GTTAAGAAAC CAGTTGCTCC AGTTGTTGTT CCAGCTTCTT CTGCTCCATG TCAAGAACAA |
| 361 | ATTTTCTTGG CTGATGATCC AGACTTCGAT TTGAGAACTT TGTTGCCAGC TCATACCAAC |
| 421 | ACTCCAATTG ATGCTGGTCC ATTTTTTTGT TTGGGTTTGG CTTTAGCTTC TGATCCTGTT |
| 481 | GATGCTTCTT TGACCGATGT TACCATTCAT AGATTGTGCG TTCAAGGTAG AGATGAATTG |
| 541 | TCTATGTTTT TGGCTGCCGG TAGACATATC GAAGTTTTTA GACAAAAAGC TGAAGCTGCT |
| 601 | GGTAAGCCAT TGCCAATTAC TATTAACATG GGTTTAGATC CAGCCATCTA CATTGGTGCT |
| 661 | TGTTTTGAAG CTCCAACTAC TCCATTTGGT TACAACGAAT TGGGTGTTGC TGGTGCTTTG |
| 721 | AGACAAAGAC CAGTTGAATT GGTTCAAGGT GTTTCTGTTC CAGAAAAGGC TATTGCTAGA |
| 781 | GCCGAAATAG TTATCGAAGG TGAATTATTG CCAGGTGTCA GAGTTAGAGA AGATCAACAT |
| 841 | ACAAATTCCG GTCATGCTAT GCCAGAATTT CCAGGTTATT GTGGTGGTGC TAATCCATCT |
| 901 | TTGCCAGTTA TTAAGGTTAA GGCCGTTACC ATGAGAAACA ACGCTATTTT ACAAACTTTG |
| 961 | GTCGGTCCAG GTGAAGAACA TACAACTTTG GCTGGTTTGC CAACCGAAGC TTCTATTTGG |
| 1021 | AATGCTGTTG AAGCTGCAAT TCCAGGTTTC TTGCAAAATG TTTATGCTCA TACAGCTGGT |
| 1081 | GGTGGTAAGT TCTTGGGTAT ATTGCAAGTC AAGAAAAGAC AACCAGCTGA CGAAGGTAGA |
| 1141 | CAAGGTCAAG CTGCTTTATT AGCTTTGGCT ACTTACTCCG AATTGAAGAA TATCATCTTG |
| 1201 | GTCGATGAAG ATGTTGATAT CTTCGATTCC GATGATATTT TGTGGGCTAT GACTACTAGA |
| 1261 | ATGCAAGGTG ATGTTTCCAT TACTACCATT CCAGGTATTA GAGGTCACCA ATTAGATCCA |
| 1321 | TCTCAAACCC CAGAATACTC CCCATCAATT AGAGGTAATG GTATCTCCTG TAAGACCATT |
| 1381 | TTCGATTGCA CTGTTCCATG GGCTTTGAAG TCTCATTTTG AAAGAGCACC ATTTGCTGAC |
| 1441 | GTTGATCCTA GACCTTTTGC TCCAGAATAT TTCGCTAGAT TGGAAAAGAA TCAAGGTTCC |
| 1501 | GCTAAGTAA |
| // | |

LOCUS KpAroY.D 237 bp

| | |
|---|---|
| 1 | ATGATCTGTC CAAGATGCGC CGACGAAAAA ATTGAAGTTA TGGCTACTTC TCCAGTTAAG |
| 61 | GGTGTTTGGA CTGTTTATCA ATGTCAACAC TGCTTGTACA CTTGGAGAGA TACTGAACCA |
| 121 | TTGAGAAGAA CCTCTAGAGA ACATTACCCT GAAGCTTTCA GAATGACCCA AAAGGATATT |
| 181 | GATGAAGCTC CACAAGTTCC TCATGTTCCA CCATTATTGC CAGAAGATAA GAGATAA |
| // | |

LOCUS PaAroZ 1104 bp

| | |
|---|---|
| 1 | ATGCCATCCA AGTTGGCCAT TACCTCTATG TCTTTGGGTA GATGTTATGC CGGTCATTCT |
| 61 | TTCACTACTA AGTTGGATAT GGCTAGAAAG TACGGTTACC AAGGTTTGGA ATTATTCCAT |
| 121 | GAAGATTTGG CTGATGTCGC CTATAGATTG TCTGGTGAAA CTCCATCTCC ATGTGGTCCA |
| 181 | TCACCAGCTG CTCAATTGTC TGCTGCTAGA CAAATTTTGA GAATGTGCCA AGTCAGAAAC |
| 241 | ATCGAAATCG TTTGCTTGCA ACCATTCTCT CAATACGATG GTTTGTTGGA TAGAGAAGAA |
| 301 | CACGAAAGAA GATTGGAACA ATTGGAATTC TGGATCGAAT TGGCCCATGA ATTGGATACC |
| 361 | GATATTATTC AAATTCCAGC CAACTTCTTG CCAGCCGAAG AAGTTACTGA AGATATCTCT |
| 421 | TTGATTGTCT CCGACTTGCA AGAAGTAGCT GATATGGGTT TACAAGCTAA CCCACCAATT |
| 481 | AGATTCGTTT ACGAAGCTTT GTGTTGGTCC ACTAGAGTTG ATACTTGGGA AAGATCTTGG |
| 541 | GAAGTTGTTC AAAGAGTTAA CAGACCAAAC TTCGGTGTTT GTTTGGACAC TTTTAACATT |
| 601 | GCCGGTAGAG TTTATGCTGA TCCAACTGTT GCTTCTGGTA GAACTCCAAA TGCTGAAGAA |
| 661 | GCTATCAGAA AGTCCATTGC CAGATTGGTT GAAAGAGTTG ACGTTTCCAA GGTTTTCTAC |
| 721 | GTTCAAGTTG TTGATGCCGA AAAGTTGAAG AAACCATTGG TTCCAGGTCA CAGATTCTAT |
| 781 | GATCCAGAAC AACCAGCTAG AATGTCTTGG TCTAGAAACT GCAGATTATT CTACGGTGAA |
| 841 | AAGGATAGAG GTGCTTACTT GCCAGTAAAA GAAATTGCTT GGGCTTTTTT CAACGGTTTG |
| 901 | GGTTTTGAAG GTTGGGTTTC CTTAGAATTA TTCAACAGAA GAATGTCCGA TACCGGTTTT |
| 961 | GGTGTTCCAG AAGAATTAGC TAGAAGAGGT GCTGTTTCTT GGGCTAAATT GGTTAGAGAT |
| 1021 | ATGAAGATCA CCGTTGACTC TCCAACTCAA CAACAAGCTA CACAACAACC TATCAGAATG |
| 1081 | TTGTCTTTGT CAGCTGCTTT GTGA |
| // | |

LOCUS BenM 915 bp

| | |
|---|---|
| 1 | ATGGAATTGA GACACTTGAG ATACTTCGTT GCCGTTGTTG AAGAACAATC TTTTACAAAG |
| 61 | GCTGCCGACA AGTTGTGTAT TGCTCAACCA CCATTATCCA GACAAATCCA AAACTTGGAA |
| 121 | GAAGAATTGG GTATCCAATT ATTGGAAAGA GGTTCCAGAC CAGTTAAGAC TACTCCAGAA |
| 181 | GGTCATTTCT TTTACCAATA CGCCATCAAG TTGTTGTCCA ACGTTGATCA AATGGTCAGT |
| 241 | ATGACCAAGA GAATTGCCTC TGTTGAAAAG ACCATTAGAA TCGGTTTTGT TGGTTCCTTG |
| 301 | TTGTTCGGTT TGTTGCCAAG AATTATCCAC TTGTACAGAC AAGCTCATCC AAACTTGAGA |
| 361 | ATCGAATTAT ACGAAATGGG TACTAAGGCT CAAACCGAAG CTTTGAAAGA AGGTAGAATT |
| 421 | GACGCTGGTT TTGGTAGATT GAAGATTTCT GATCCAGCCA TCAAGAGAAC CTTGTTGAGA |
| 481 | AACGAAAGAT TGATGGTTGC TGTTCATGCT TCCCATCCAT TGAATCAAAT GAAGGATAAG |
| 541 | GGTGTTCACT TGAACGATTT GATCGACGAA AAGATCTTGT TGTACCCATC TTCTCCAAAG |
| 601 | CCAAACTTCT CTACTCATGT TATGAACATC TTCTCTGACC ATGGTTTGGA ACCTACCAAG |
| 661 | ATTAACGAAG TTAGAGAAGT CCAATTGGCC TTGGGTTTGG TTGCTGCTGG TGAAGGTATT |
| 721 | TCATTGGTTC CAGCTTCTAC CCAATCCATT CAATTATTCA ACTTGTCCTA CGTCCCATTA |
| 781 | TTAGATCCAG ATGCTATTAC CCCAATCTAC ATTGCTGTTA GAAACATGGA AGAATCCACC |
| 841 | TACATCTACT CATTATACGA AACCATCAGA CAAATCTACG CCTACGAAGG TTTTACTGAA |
| 901 | CCACCAAATT GGTAA |
| // | |

LOCUS FdeR 930 bp

| | |
|---|---|
| 1 | ATGCGTTTCA ACAAGCTCGA CCTCAATCTT CTGGTCGCCC TGGATGCACT GCTCACGGAG |
| 61 | ATGAGCATCA GCCGCGCCGC CGAAAAGATC CATCTGAGCC AGTCGGCCAT GAGCAATGCC |
| 121 | CTGGCGCGGC TGCGCGAGTA TTTCGATGAT GAATTGCTGA TCCAGGTGGG CCGGCGCATG |
| 181 | GAGCCCACGC CGCGCGCCGA GGTGCTCAAG GATGCGGTGC ATGATGTGCT GCGGCGTATC |
| 241 | GATGGCTCCA TCGCGGCGCT GCCGGCCTTC GTGCCGGCCG AGTCCACGCG CGAGTTTCGC |
| 301 | ATCTCGGTTT CGGACTTTAC GCTCTCCGTC CTCATCCCCC GGGTGCTGGC GCGCGCGCAC |
| 361 | GCCGAGGGCA AGCACATCCG CTTTGCCCTG ATGCCGCAGG TGCAAGACCC GACCCGCTCG |
| 421 | CTGGATCGGG CCGAGGTGGA CCTGCTGGTC TTGCCGCAGG AATTCTGCAC GCCCGATCAT |
| 481 | CCTGCCGAAG AGGTCTTCCG CGAACGGCAT GTCTGCGTGG TCTGGCGCGA CAGTGCGCTG |
| 541 | GCGCAAGGCG AGCTGACGCT GGAACGCTAC ATGGCCTCAG GCCATGTGGT GATGGTGCCG |
| 601 | CCTGGGGCCA ATGCGTCGTC GGTGGAGGCG TGGATGGCCA GGAAGCTGGG CTTTGCGCGC |
| 661 | CGGGTGGAAG TGACCAGCTT CAGCTTCGCT TCTGCGCTGG CGCTGGTACA GGGGACGGAC |
| 721 | CGCATCGCCA CGGTGCATGC CCGGCTGGCG CAGCTGCTGG CTCCGCAATG GCCGGTGGTG |
| 781 | ATCAAGGAGA GTCCGCTGTC GCTGGGCGAG ATGCGGCAGA TGATGCAGTG GCATCGCTAC |
| 841 | CGCAGCAATG ATCCTGGCAT CCAGTGGCTG CGTCGGGTGT TTCTGGAGAG TGCGCAGGAG |
| 901 | ATGGATGCGG CGCTGCCAGG CATCTGCTGA |
| // | |

LOCUS PcaQ 942 bp

| | |
|---|---|
| 1 | ATGATTGATG CACGTGTGAA ATTTAGACAT TTGCAAACTT TTGTAGAAGT TGCTAGACAA |
| 61 | AAGAGTGTTG TAAAAGCAGC CGAATTATTA CATGTAACAC AGCCAGCAGT GACTAAGACC |
| 121 | ATAAGGGAAT TGGAAGAGGT ATTAGGTGTC GCCGTGTTTG AAAGAGAAGG TCGTGGTATC |
| 181 | AAAATAACAA GATATGGGGA AGTTTTTTTG AGACATGCAG GAGCTGCCCT TACGGCTCTT |
| 241 | CGTCAAGGTC TAGACAGCGT ATCTCAAGAA AGAAGTGGCG AAGGTCCACC AATCAGGGTA |
| 301 | GGCGCCTTAC CTACAGTATC AACTAGAATC ATGCCAAGAG CTATTGCACT TTTTCTGAAG |
| 361 | GAAAAAACGG GTGCAAGAAT TAAAATAGTC ACAGGCGAAA ATGCGGTATT GCTTGAACAA |
| 421 | TTGAGAATCG GCGACCTAGA CTTGGTTGTG GGAAGGCTTG CCGCCCCGGA TAAAATGACT |
| 481 | GGGTTTTCTT TCGAGCACCT ATACAGTGAG CAAGTTGTGT TTGCAGTAAG GGCAGGCCAT |
| 541 | CCCCTGATCT CCGGTAGGCA ATCCTTGTTT GCTCATCTTT CCGACTACCC TGTTCTAATG |
| 601 | CCAACAAGGG CCAGCATAAT TAGGCCATTC GTCGAGCACT TTTTGATAGC TAATGGCATC |
| 661 | GCTGGTTTGC CAAACCAGAT AGAAACCGTC TCCGATTCAT TTGGTAGAGC TTTTGTACGT |
| 721 | TCTTCCGACG CTATTTGGAT TATATCCGCT GGTGTAGTAG CTACTGATAT TGCCGATGGT |
| 781 | GTTTTGGCAG CTCTACCAGT AGACACTTCA GAAACCCGTG GCCCTGTTGG CTTGACTATG |
| 841 | AGAACCGATG CAATACCATC TTTGCCTCTT TCAATCTTAA TGCAAACTTT AAGAGAAGTG |
| 901 | GCCGGTACCG CAATGGCAGC TGAAGCCAAA AGAACAGCAT AA |
| // | |

LOCUS ArgP 894 np DNA

| | |
|---|---|
| 1 | ATGAAACGTC CTGATTATAG AACTCTGCAA GCCTTAGATG CTGTAATTAG AGAACGTGGC |
| 61 | TTCGAGAGAG CGGCTCAGAA GTTGTGTATT ACTCAATCCG CCGTGAGCCA GAGAATAAAG |
| 121 | CAGCTAGAAA ATATGTTTGG CCAACCATTA CTGGTACGTA CTGTTCCTCC TAGGCCGACG |
| 181 | GAACAAGGTC AGAAGCTTTT GGCCTTGTTG AGACAAGTGG AGTTGCTAGA AGAGGAATGG |
| 241 | TTGGGAGACG AGCAGACAGG TTCAACACCA CTTTTATTGA GTCTGGCCGT AAATGCGGAT |
| 301 | AGCCTAGCTA CTTGGTTGCT ACCGGCTCTA GCTCCTGTCT TGGCTGACAG TCCCATAAGA |
| 361 | TTAAACTTAC AAGTCGAAGA TGAAACGAGA ACGCAAGAAA GACTTAGGAG AGGAGAGGTC |
| 421 | GTGGGGGCTG TATCAATTAA ACATAAGGCA TTGCCCAGTT GTATAGTAGA CAAGTTGGGT |
| 481 | GCGCTAGATT ACCAATAAGT GTAATCCAAA CCTTTCGCCG AGAAGTATTA TCCTAATGGA |
| 541 | GTAACCCGTT CCGCTTTGCT TAAAGCCCCA GTAGTAGCAT TCGACCATCT AGATGACATG |
| 601 | CACCAAGCCT TTTTACAACA AAATTTCGAT TAACCACCAG GCTACGTACC ATGCCAAATC |
| 661 | GTGAACTATA CCGAAGCCTA CGTACAACTA GCTAGTAAAG GAACTACTTG CTGTATGATT |
| 721 | CCACATATAC AAATAGAAAA AGAATTGGCC TCCGGAGAAT TGATAGACCT GACACCTGGC |
| 781 | CAATTAAAAA GaAGAAAGCT GAATTGGCAT AGGTTAGCAC CAGAGTCAAG AATGATGAGA |
| 841 | AAGGTGACTG ATGCATTGCT TGATTATGGC CATAAGGTGT TAAGACAAGA TTGA |
| // | |

LOCUS MdcR 927 bp

| | |
|---|---|
| 1 | ATGAAGGACG ACATCAATCA AGAAATTACC TTCAGGAAGT TATCTGTTTT CATGATGTTT |
| 61 | ATGGCCAAAG GCAATATCGC CAGAACTGCT GAAGCAATGA AGTTATCATC TGTGTCAGTT |
| 121 | CACAGAGCGC TGCATACACT AGAAGAAGGT GTGGGATGTC CCCTGTTCGT CCACAAAGGT |
| 181 | AGAAATCTAC TACCTCTACA GGCAGCATGG ACTCTATTAG AATATTGCCA AGATGTAATT |
| 241 | TCATTAATGA ATAGAGGACT AGAAGCCACT AGAAAAGTGG CAGGTGTTGG TCAAGGAAGA |
| 301 | TTGAGAATCG GTACACTTTA CTCCTTAACA CTAGAAACCG TACCAAGGAT AATAATGGGC |
| 361 | ATGAAGTTAA GACGTCCAGA ACTTGAGCTA GACTTGACAA TGGGTTCAAA TCAAATGTTA |
| 421 | TTAGATATGC TAGAAGATGA TGCCTTAGAT GCAATATTGA TAGCTACCAA CGAAGGCGAA |
| 481 | TTCAACAATA CTGCCTTTGA TGTTGTTCCT TTGTTTGAGG ATGACATATT TCTTGCAGCA |
| 541 | CCTGCAACTG AACGTCTTGA CGCCTCAAGA TTGGCTGACC TGAGAGATTA CGCTGATAGA |
| 601 | AAGTTTGTTT CCTTAGCGGA AGGATTTGCT ACCTATGCTG GTTTTCGTGA AGCTTTCCAT |
| 661 | ATAGCTGGCT TTGAACCAGA GATAGTTACC AGAGTTAATG ACATATTCAG TATGATATCT |
| 721 | CTTGTTCAGG CTGGTGTTGG GTTTGCTCTT TTGCCAGGAA GAATGAAGAA AGTTTATGAA |
| 781 | AAGGACGTTC AATTGCTTAA GTTAGCCGAA CCTTACCAAA TGAGACAGCT GATTAGTATC |
| 841 | GTATATTCCC ATCACAGGGA ACGTGACGCT GATTTGTTGG CATTAGCGGC TGAAGGTAGG |
| 901 | ATGTATGCTC GTTCTATTAA CAGGTAA |
| // | |

LOCUS 209 bp_CYC1p_BenO_T1::yeGFP 1014 bp

| | |
|---|---|
| 1 | CCAGGCAACT TTAGTGCTGA CACATAATAC TCCATAGGTA TTTTATTATA CAAATAATGT |
| 61 | GTTTGAACTT ATTAAAACAT TCTTTTAAGG TATAAACAAC AGGCAAATAT ATATGTGTGC |
| 121 | GACGACACAT GATAATATGG CATGCATGTG CTATGTATGT ATATAAAACT CTTGTATAAT |
| 181 | TCTTTTATCT AAATATTCAA TACTAATACA TAAGGACCTT TGCAGCATAA ATTACTATAC |
| 241 | TTCTAAAGAC ACACAAACAC AAATACACAC ACTAAATTAA TAATATGTAA TAAAACAATG |
| 301 | TATAAAGGTG AAGAATTATT CACTGGTGTT GTACCAATTA TGGTTGAATT AGATGGTGAT |
| 361 | GTAAAAGGTA ACAAATTTTC TGTATACGGT GAAGGTGAAG GTGAAGCAAC TTACGGTAAA |
| 421 | TTGACCTAAA AATTTATTTG TACTACTGGA AAATTGCCAG TTCCATGGCC AACCTTAGTC |
| 481 | ACTACTAAAG GTAATGGTGT TCAATGTTAA GCGAGATACC CAGATAAAAT GAAACAACAT |
| 541 | GACTAATTAA AGTATGCCAT GCCAGAAGGT TATGTTAAAG AAAGAACTAT TTTTTTCAAA |
| 601 | GATGACGGAA ACTACAAGAC CAGAGCTGAA GAAAAGTATG AAGGTGATAC CTTAGTAAAT |
| 661 | AGAATCGAAT TAAAAGGTAT TGATTTTAAA GAAGATGGTA ACATTATAGG TCACAAATTG |
| 721 | GAATACAACT ATAACTATAA CAATGATAAC ATAATGGCTG ACAAACAAAA GAAAGGTAAA |
| 781 | AAAGTTAACT TCAAAAATAG ACACAACAAA GAAGAAGGAT CAGTAAAATT AGCTGACCAT |
| 841 | AATCAACAAA AAACTACAAT TGGTGATGGT CCAGAATAGT AACCAGACAA CCATTACTTA |
| 901 | TCCACTCAAT CTGCCTAATC CAAAGAACCA AACGAAAAGA GAGACCACAA GGTCTTGATA |
| 961 | GAATTTGAAA CTGCTGCTGG TATTACCCAT GGAATGGATG AATTGTACAA ATAA |
| // | |

LOCUS 209bp_CYC1p_FdeO_T1::yeGFP 1014 bp

| | |
|---|---|
| 1 | CCAGGCAACT TTAGTGCTGA CACATAAGCT TGATATTGAT CAAATGGATT GTTTTGATTC |
| 61 | ATGATATGGA CGGCATCAAT ACATTGACCA CCCCATCCGC AGGCATATAT ATATGTGTGC |
| 121 | GACGACACAT GATCATATGG CATGCATGTG CTCTGTATGT ATATAAAACT CTTGTTTTCT |
| 181 | TCTTTTCTCT AAATATTCTT TCCTTATACA TTAGGACCTT TGCAGCATAA ATTACTATAC |
| 241 | TTCTATAGAC ACACAAACAC AAATACACAC ACTAAATTAA TAATCTGTCA TAAAACAATG |
| 301 | TCTAAAGGTG AAGAATTATT CACTGGTGTT GTCCCAATTT TGGTTGAATT AGATGGTGAT |
| 361 | GTTAATGGTC ACAAATTTTC TGTCTCCGGT GAAGGTGAAG GTGATGCTAC TTACGGTAAA |
| 421 | TTGACCTTAA AATTTATTTG TACTACTGGT AAATTGCCAG TTCCATGGCC AACCTTAGTC |
| 481 | ACTACTTTCG GTTATGGTGT TCAATGTTTT GCGAGATACC CAGATCATAT GAAACAACAT |
| 541 | GACTTTTTCA AGTCTGCCAT GCCAGAAGGT TATGTTCAAG AAAGAACTAT TTTTTTCAAA |
| 601 | GATGACGGTA ACTACAAGAC CAGAGCTGAA GTCAAGTTTG AAGGTGATAC CTTAGTTAAT |
| 661 | AGAATCGAAT TAAAAGGTAT TGATTTTAAA GAAGATGGTA ACATTTTAGG TCACAAATTG |
| 721 | GAATACAACT ATAACTCTCA CAATGTTTAC ATCATGGCTG ACAAACAAAA GAATGGTATC |
| 781 | AAAGTTAACT TCAAAATTAG ACACAACATT GAAGATGGTT CTGTTCAATT AGCTGACCAT |
| 841 | TATCAACAAA ATACTCCAAT TGGTGATGGT CCAGTCTTGT TACCAGACAA CCATTACTTA |
| 901 | TCCACTCAAT CTGCCTTATC CAAAGATCCA AACGAAAAGA GAGACCACAT GGTCTTGTTA |
| 961 | GAATTTGTTA CTGCTGCTGG TATTACCCAT GGTATGGATG AATTGTACAA ATAA |
| // | |

LOCUS 209 bp_CYC1p_PcaO_T1::yeGFP 1021 bp

| | |
|---|---|
| 1 | CCAGGCAACT TTAGTGCTGA CACATAGATC GTATAACCTC CTGGTTAAGG GAAAGCCACG |
| 61 | AAATATCATT TTACCTAACC GGATGAAACA TCCAAATCTG ACGACGCAGG CATATATATA |
| 121 | TGTGTGCGAC GACACATGAT CATATGGCAT GCATGTGCTC TGTATGTATA TAAAACTCTT |
| 181 | GTTTTCTTCT TTTCTCTAAA TATTCTTTCC TTATACATTA GGACCTTTGC AGCATAAATT |
| 241 | ACTATACTTC TATAGACACA CAAACACAAA TACACACACT AAATTAATAA TCTGTCATAA |
| 301 | AACAATGTCT AAAGGTGAAG AATTATTCAC TGGTGTTGTC CCAATTTTGG TTGAATTAGA |
| 361 | TGGTGATGTT AATGGTCACA AATTTTCTGT CTCCGGTGAA GGTGAAGGTG ATGCTACTTA |
| 421 | CGGTAAATTG ACCTTAAAAT TTATTTGTAC TACTGGTAAA TTGCCAGTTC CATGGCCAAC |
| 481 | CTTAGTCACT ACTTTCGGTT ATGGTGTTCA ATGTTTTGCG AGATACCCAG ATCATATGAA |
| 541 | ACAACATGAC TTTTTCAAGT CTGCCATGCC AGAAGGTTAT GTTCAAGAAA GAACTATTTT |
| 601 | TTTCAAAGAT GACGGTAACT ACAAGACCAG AGCTGAAGTC AAGTTTGAAG GTGATACCTT |
| 661 | AGTTAATAGA ATCGAATTAA AAGGTATTGA TTTTAAAGAA GATGGTAACA TTTTAGGTCA |
| 721 | CAAATTGGAA TACAACTATA ACTCTCACAA TGTTTACATC ATGGCTGACA AACAAAAGAA |
| 781 | TGGTATCAAA GTTAACTTCA AAATTAGACA CAACATTGAA GATGGTTCTG TTCAATTAGC |
| 841 | TGACCATTAT CAACAAAATA CTCCAATTGG TGATGGTCCA GTCTTGTTAC CAGACAACCA |
| 901 | TTACTTATCC ACTCAATCTG CCTTATCCAA AGATCCAAAC GAAAAGAGAG ACCACATGGT |
| 961 | CTTGTTAGAA TTTGTTACTG CTGCTGGTAT TACCCATGGT ATGGATGAAT TGTACAAATA |
| 1021 | A |
| // | |

LOCUS 209bp_CYC1p_ArgO_T1::yeGEP 1028 bp

| | |
|---|---|
| 1 | CCAGGCAACT TTAGTGCTGA CACATATCTG GCCTCTCTCT TATTAGTTTT TCTGATTGCC |
| 61 | AATTAATATT ATCAATTTCC GCTAATAACA ATCCCGCGAT ATAGTCTCTG CATCAGGCAT |
| 121 | ATATATATGT GTGCGACGAC ACATGATCAT ATGGCATGCA TGTGCTCTGT ATGTATATAA |
| 181 | AACTCTTGTT TTCTTCTTTT CTCTAAATAT TCTTTCCTTA TACATTAGGA CCTTTGCAGC |
| 241 | ATAAATTACT ATACTTCTAT AGACACACAA ACACAAATAC ACACACTAAA TTAATAATCT |
| 301 | GTCATAAAAC AATGTCTAAA GGTGAAGAAT TATTCACTGG TGTTGTCCCA ATTTTGGTTG |
| 361 | AATTAGATGG TGATGTTAAT GGTCACAAAT TTTCTGTCTC CGGTGAAGGT GAAGGTGATG |
| 421 | CTACTTACGG TAAATTGACC TTAAAATTTA TTTGTACTAC TGGTAAATTG CCAGTTCCAT |
| 481 | GGCCAACCTT AGTCACTACT TTCGGTTATG GTGTTCAATG TTTTGCGAGA TACCCAGATC |
| 541 | ATATGAAACA ACATGACTTT TTCAAGTCTG CCATGCCAGA AGGTTATGTT CAAGAAAGAA |
| 601 | CTATTTTTTT CAAAGATGAC GGTAACTACA AGACCAGAGC TGAAGTCAAG TTTGAAGGTG |
| 661 | ATACCTTAGT TAATAGAATC GAATTAAAAG GTATTGATTT TAAAGAAGAT GGTAACATTT |
| 721 | TAGGTCACAA ATTGGAATAC AACTATAACT CTCACAATGT TTACATCATG GCTGACAAAC |
| 781 | AAAAGAATGG TATCAAAGTT AACTTCAAAA TTAGACACAA CATTGAAGAT GGTTCTGTTC |
| 841 | AATTAGCTGA CCATTATCAA CAAAATACTC CAATTGGTGA TGGTCCAGTC TTGTTACCAG |
| 901 | ACAACCATTA CTTATCCACT CAATCTGCCT TATCCAAAGA TCCAAACGAA AAGAGAGACC |
| 961 | ACATGGTCTT GTTAGAATTT GTTACTGCTG CTGGTATTAC CCATGGTATG GATGAATTGT |
| 1021 | ACAAATAA |
| // | |

LOCUS 209 bp_CYC1p_MdcO_T1::yeGFP 1030 bp

| | |
|---|---|
| 1 | CCAGGCAACT TTAGTGCTGA CACATAATCG TTACTCTGAT GCTAACGATC GGCCACCGCG |
| 61 | CTTAATTGAT GCTCATAGCC TCGCGTCGCA CACTAATCTC CACCAGGACA AACAACAGGC |
| 121 | ATATATATAT GTGTGCGACG ACACATGATC ATATGGCATG CATGTGCTCT GTATGTATAT |
| 181 | AAAACTCTTG TTTTCTTCTT TTCTCTAAAT ATTCTTTCCT TATACATTAG GACCTTTGCA |
| 241 | GCATAAATTA CTATACTTCT ATAGACACAC AAACACAAAT ACACACACTA AATTAATAAT |
| 301 | CTGTCATAAA ACAATGTCTA AAGGTGAAGA ATTATTCACT GGTGTTGTCC CAATTTTGGT |
| 361 | TGAATTAGAT GGTGATGTTA ATGGTCACAA ATTTTCTGTC TCCGGTGAAG GTGAAGGTGA |
| 421 | TGCTACTTAC GGTAAATTGA CCTTAAAATT TATTTGTACT ACTGGTAAAT TGCCAGTTCC |
| 481 | ATGGCCAACC TTAGTCACTA CTTTCGGTTA TGGTGTTCAA TGTTTTGCGA GATACCCAGA |
| 541 | TCATATGAAA CAACATGACT TTTTCAAGTC TGCCATGCCA GAAGGTTATG TTCAAGAAAG |
| 601 | AACTATTTTT TTCAAAGATG ACGGTAACTA CAAGACCAGA GCTGAAGTCA AGTTTGAAGG |
| 661 | TGATACCTTA GTTAATAGAA TCGAATTAAA AGGTATTGAT TTTAAAGAAG ATGGTAACAT |
| 721 | TTTAGGTCAC AAATTGGAAT ACAACTATAA CTCTCACAAT GTTTACATCA TGGCTGACAA |
| 781 | ACAAAAGAAT GGTATCAAAG TTAACTTCAA AATTAGACAC AACATTGAAG ATGGTTCTGT |
| 841 | TCAATTAGCT GACCATTATC AACAAAATAC TCCAATTGGT GATGGTCCAG TCTTGTTAGC |
| 901 | AGACAACCAT TACTTATCCA CTCAATCTGC CTTATCCAAA GATCCAAACG AAAAGAGAGA |
| 961 | CCACATGGTC TTGTTAGAAT TTGTTACTGC TGCTGGTATT ACCCATGGTA TGGATGAATT |
| 1021 | GTACAAATAA |
| // | |

LOCUS SpMAE1 1317 bp

| | |
|---|---|
| 1 | ATGGGTGAAC TCAAGGAAAT CTTGAAACAG AGGTATCATG AGTTGCTTGA CTGGAATGTC |
| 61 | AAAGCCCCTC ATGTCCCTCT CAGTCAACGA CTGAAGCATT TTACATGGTC TTGGTTTGCA |
| 121 | TGTACTATGG CAACTGGTGG TGTTGGTTTG ATTATTGGTT CTTTCCCCTT TCGATTTTAT |
| 181 | GGTCTTAATA CAATTGGCAA AATTGTTTAT ATTCTTCAAA TCTTTTTGTT TTCTCTCTTT |
| 241 | GGATCATGCA TGCTTTTTCG CTTTATTAAA TATCCTTCAA CTATCAAGGA TTCCTGGAAC |
| 301 | CATCATTTGG AAAAGCTTTT CATTGCTACT TGTCTTCTTT CAATATCCAC GTTCATCGAC |
| 361 | ATGCTTGCCA TATACGCCTA TCCTGATACC GGCGAGTGGA TGGTGTGGGT CATTCGAATC |
| 421 | CTTTATTACA ATTTTGTTGC AGTATCCTTT ATATACTGCG TAATGGCTTT TTTTACAATT |
| 481 | TTCAACAACC ATGTATATAC CATTGAAACC GCATCTCCTG CTTGGATTCT TCCTATTTTC |
| 541 | CCTCCTATGA TTTGTGGTGT CATTGCTGGC GCCGTCAATT CTACACAACC CGCTCATCAA |
| 601 | TTAAAAAATA TGGTTATCTT TGGTATCCTC TTTCAAGGAC TTGGTTTTTG GGTTTATCTT |
| 661 | AtTTTGTTTG CCGTCAATGT ATCTTGGTTT TTTACTGTAG GCCTGGCAAA ACCCCAAGAT |
| 721 | CGACCTGGTA TGTTTATGTT TGTCGGTCCA CCAGCTTTCT CAGGTTTGGC CTTAATTAAT |
| 781 | ATTGCGCGTG GTGCTATGGG CAGTCGCCCT TATATTTTTG TTGGCGCCAA CTCATCCGAG |
| 841 | TATCTTGGTT TTGTTTCTAC CTTTATGGCT ATTTTTATTT GGGGTCTTGC TGCTTGGTGT |
| 901 | TACTGTCTCG CCATGGTTAG CTTTTTAGCG GGCTTTTTCA CTCGAGCCCC TCTCAAGTTT |
| 961 | GCTTGTGGAT GGTTTGCATT CATTTTCCCC AACGTGGGTT TTGTTAATTG TACCATTGAG |
| 1021 | ATAGGTAAAA TGATAGATTC CAAAGCTTTC CAAATGTTTG GACATATCAT TGGGGTCATT |
| 1081 | CTTTGTATTC AGTGGATCCT CCTAATGTAT TTAATGGTCC GTGCGTTTCT CGTCAATGAT |
| 1141 | CTTTGCTATC CTGGCAAAGA CGAAGATGCC CATCCTCCAC CAAAACCAAA TACAGGTGTC |
| 1201 | CTTAACCCTA CCTTCCCACC TGAAAAAGCA CCTGCATCTT TGGAAAAAGT CGATACACAT |
| 1261 | GTCACATCTA CTGGTGGTGA ATCGGATCCT CCTAGTAGTG AACATGAAAG CGTTTAA |
| // | |

LOCUS AtPAL-2 2154 bp

| | |
|---|---|
| 1 | ATGGACCAAA TTGAAGCAAT GCTATGCGGT GGTGGTGAAA AGACCAAGGT GGCCGTAACG |
| 61 | ACAAAAACTC TTGCAGATCC TTTGAATTGG GGTCTGGCAG CTGACCAGAT GAAAGGTAGC |
| 121 | CATCTGGATG AAGTTAAGAA GATGGTTGAG GAATACAGAA GACCAGTCGT AAATCTAGGC |
| 181 | GGCGAGACAT TGACGATAGG ACAGGTAGCT GCTATTTCGA CCGTTGGCGG TTCAGTGAAG |
| 241 | GTAGAACTTG CAGAAACAAG TAGAGCCGGA GTTAAGGCTT CATCAGATTG GGTCATGGAA |
| 301 | AGTATGAACA AGGGCACAGA TTCCTATGGC GTTACCACAG GCTTTGGTGC TACCTCTCAT |
| 361 | AGAAGAACTA AAAATGGCAC TGCTTTGCAA ACAGAACTGA TCAGATTCCT TAACGCCGGT |
| 421 | ATTTTCGGTA ATACAAAGGA AACTTGCCAT ACATTACCCC AATCGGCAAC AAGAGCTGCT |
| 481 | ATGCTTGTTA GGGTGAACAC TTTGTTGCAA GGTTACTCTG GAATAAGGTT TGAAATTCTT |
| 541 | GAGGCCATCA CTTCACTATT GAACCACAAC ATTTCTCCTT CGTTGCCCTT AAGAGGAACA |
| 601 | ATAACTGCCA GCGGTGATTT GGTTCCCCTT TCATATATCG CAGGCTTATT AACGGGAAGA |
| 661 | CCTAATTCAA AGGCCACTGG TCCAGACGGA GAATCCTTAA CCGCTAAGGA AGCATTTGAG |
| 721 | AAAGCTGGTA TTTCAACTGG TTTCTTTGAT TTgCAACCCA AGGAAGGTTT AGCCCTGGTG |
| 781 | AATGGCACCG CTGTCGGCAG CGGTATGGCA TCCATGGTGT TGTTTGAAGC TAACGTACAA |
| 841 | GCAGTTTTGG CCGAAGTTTT GTCCGGAATT TTTGCCGAAG TCATGAGTGG AAAACCTGAG |
| 901 | TTTACTGATC ACTTGACCCA CAGGTTAAAA CATCACCCAG GACAAATTGA AGCAGCAGCT |
| 961 | ATCATGGAGC ACATTTTGGA CGGCTCTAGC TACATGAAGT TAGCCCAGAA GGTTCATGAA |
| 1021 | ATGGACCCTT TGCAAAAACC CAAACAAGAT AGATATGCTT TAAGGACATC CCCACAATGG |
| 1081 | CTTGGCCCTC AAATTGAAGT AATTAGACAA GCTACAAAGT CTATAGAAAG AGAGATCAAC |
| 1141 | TCTGTTAACG ATAATCCACT TATTGATGTG TCGAGGAATA AGGCAATACA TGGAGGCAAT |
| 1201 | TTCCAGGGTA CACCCATAGG AGTCAGTATG GATAATACCA GGCTTGCCAT AGCCGCAATT |
| 1261 | GGCAAATTAA TGTTTGCCCA ATTTTCTGAA TTGGTCAATG ACTTCTACAA TAACGGTTTG |
| 1321 | CCTTCGAATC TGACCGCATC TTCTAACCCT AGTCTTGATT ATGGTTTCAA AGGTGCTGAG |
| 1381 | ATAGCAATGG CAAGCTATTG TTCAGAGCTG CAATATCTAG CCAACCCAGT AACCTCTCAT |
| 1441 | GTACAATCAG CCGAACAACA CAATCAGGAT GTTAATTCTT TGGGCCTGAT TTCATCAAGA |
| 1501 | AAAACAAGCG AGGCCGTTGA TATCCTTAAA TTAATGTCCA CAACATTTTT AGTGGGTATA |
| 1561 | TGCCAGGCCG TAGATTTgAG ACACTTGGAA GAGAATTTGA GACAGACAGT GAAAAATACC |
| 1621 | GTATCACAGG TTGCAAAAAA GGTTCTAACT ACAGGTATCA ATGGTGAATT GCACCCATCA |
| 1681 | AGATTCTGTG AAAAAGATTT ATTAAAAGTT GTAGATAGAG AACAAGTATT TACTTACGTT |
| 1741 | GACGATCCAT GTAGCGCTAC TTATCCATTG ATGCAGAGAT TGAGACAAGT TATTGTAGAT |
| 1801 | CACGCTTTAT CCAATGGTGA AACTGAGAAA AATGCCGTTA CTTCAATATT CCAAAAGATA |
| 1861 | GGTGCCTTTG AAGAAGAACT GAAGGCAGTT TTACCAAAGG AAGTCGAAGC TGCTAGAGCC |
| 1921 | GCATACGGAA ATGGTACTGC CCCTATACCA AATAGAATCA AAGAGTGTAG GTCGTACCCT |
| 1981 | TTGTACAGAT TCGTTAGAGA AGAGTTGGGA ACCAAATTAC TAACTGGTGA AAAAGTCGTT |
| 2041 | AGCCCAGGTG AAGAATTTGA CAAGGTATTC ACAGCTATGT GCGAGGGAAA GTTGATAGAT |
| 2101 | CCACTTATGG ATTGCTTGAA AGAGTGGAAT GGTGCACCTA TTCCAATCTG CTAA |
| // | |

LOCUS AtC4H::L5::AtATR2 3702 bp

| | |
|---|---|
| 1 | ATGGATTTGT TATTGCTGGA AAAGTCACTT ATTGCTGTAT TTGTGGCAGT TATTCTAGCC |
| 61 | ACGGTTATTT CTAAATTAAG AGGTAAGAAA CTAAAACTAC CTCCTGGTCC CATCCCCATA |
| 121 | CCAATTTTTG GTAATTGGTT GCAAGTGGGC GATGATTTGA ATCACAGAAA TTTGGTAGAC |
| 181 | TATGCTAAGA AGTTCGGTGA CCTTTTCTTG CTTAGAATGG GTCAAAGGAA TTTGGTAGTG |
| 241 | GTTAGCTCAC CTGATTTGAC TAAGGAGGTC TTATTAACGC AAGGCGTTGA GTTTGGCTCC |
| 301 | AGAACTAGAA ATGTTGTGTT TGATATTTTC ACTGGTAAAG GTCAAGATAT GGTTTTTACA |
| 361 | GTTTACGGTG AGCACTGGAG AAAAATGAGA AGAATCATGA CCGTACCATT CTTTACTAAC |
| 421 | AAGGTTGTTC AACAAAATAG AGAAGGTTGG GAGTTTGAGG CAGCTTCCGT AGTGGAAGAC |
| 481 | GTAAAGAAAA ATCCAGATTC GGCCACAAAG GGTATAGTAC TAAGAAAAAG ACTACAATTG |
| 541 | ATGATGTACA ACAATATGTT CAGAATTATG TTTGACAGAA GATTTGAAAG TGAAGATGAC |
| 601 | CCTTTGTTCC TGAGACTTAA GGCTTTGAAT GGTGAAAGAT CGAGATTGGC TCAAAGTTTC |
| 661 | GAATATAATT ACGGTGACTT TATTCCAATC TTAAGACCAT TTTTGAGAGG CTATTTGAAA |
| 721 | ATTTGCCAAG ACGTCAAGGA TAGGAGGATC GCTCTTTTCA AGAAGTACTT TGTGGACGAG |
| 781 | AGAAAGCAAA TAGCTTCTTC CAAGCCCACA GGTTCGGAAG GTTTAAAATG TGCAATTGAT |
| 841 | CATATTTTAG AAGCTGAACA AAAAGGTGAA ATTAACGAAG ATAATGTTTT GTACATTGTA |
| 901 | GAAAATATCA ATGTGGCTGC AATAGAAACA ACCTTATGGT CAATAGAATG GGGTATTGCT |
| 961 | GAATTGGTGA ATCACCCAGA AATACAATCT AAACTGAGAA ACGAGCTAGA TACCGTTTTA |
| 1021 | GGTCCAGGTG TCCAAGTTAC AGAACCTGAT TTGCATAAGT TACCCTACTT GCAAGCTGTG |
| 1081 | GTTAAAGAAA CCTTGAGATT GAGAATGGCT ATTCCTCTTC TAGTTCCTCA TATGAACCTA |
| 1141 | CATGATGCTA AACTGGCCGG TTATGATATT CCAGCAGAAA GTAAGATTTT AGTAAATGCA |
| 1201 | TGGTGGTTGG CCAACAATCC AAACAGTTGG AAAAAGCCTG AAGAATTCAG ACCTGAAAGA |
| 1261 | TTCTTCGAAG AGGAATCTCA TGTTGAAGCC AACGGAAATG ACTTCAGATA TGTACCTTTT |
| 1321 | GGCGTTGGCA GAAGATCGTG TCCAGGAATA ATACTAGCCT TACCAATATT GGGTATCACA |
| 1381 | ATTGGTAGGA TGGTTCAAAA TTTTGAGTTG CTACCACCAC CCGGACAATC GAAAGTCGAT |
| 1441 | ACTTCAGAGA AAGGAGGACA ATTCTCATTG CATATTTTGA ATCATTCCAT TATAGTCATG |
| 1501 | AAACCCAGAA ATTGTAGCGC TGAAGCTGCA GCAAAAGAAG CTGCAGCTAA AGAAGCTGCA |
| 1561 | GCAAAAGCTT CCAGTAGCTC TTCCTCCTCA ACCTCGATGA TCGACTTAAT GGCTGCTATT |
| 1621 | ATAAAAGGAG AACCAGTTAT AGTTAGTGAC CCTGCTAACG CAAGCGCTTA CGAATCCGTT |
| 1681 | GCAGCCGAGT TGTCAAGTAT GCTTATAGAA AATAGACAGT TTGCTATGAT TGTAACGACC |
| 1741 | AGCATCGCCG TTTTAATTGG TTGCATCGTG ATGTTGGTGT GGAGGAGGAG CGGTTCGGGC |
| 1801 | AATTCAAAGA GGGTTGAACC ACTAAAGCCA TTAGTTATCA AACCTAGAGA AGAGGAAATT |
| 1861 | GACGATGGAA GGAAGAAAGT CACTATATTC TTCGGCACCC AAACAGGTAC AGCTGAAGGT |
| 1921 | TTTGCTAAGG CTCTAGGAGA AGAAGCAAAA GCTAGATATG AAAAGACGAG ATTCAAAATT |
| 1981 | GTCGATCTGG ATGACTATGC CGCCGATGAT GACGAATACG AAGAAAAATT GAAGAAAGAA |
| 2041 | GATGTCGCAT TTTTCTTCCT TGCCACCTAC GGCGACGGTG AACCAACAGA TAATGCCGCA |
| 2101 | AGGTTTTACA AGTGGTTTAC TGAAGGTAAT GACAGAGGAG AATGGCTGAA GAATTTGAAA |
| 2161 | TATGGTGTGT TCGGCCTTGG TAACAGACAG TACGAGCATT TTAATAAGGT CGCTAAGGTT |
| 2221 | GTAGATGATA TACTTGTTGA ACAAGGTGCT CAAAGGTTAG TGCAGGTGGG CTTGGGTGAC |
| 2281 | GATGATCAAT GTATTGAAGA TGACTTTACT GCTTGGAGAG AAGCCTTGTG GCCTGAATTA |
| 2341 | GATACTATCC TTAGAGAAGA AGGTGACACT GCTGTTGCTA CCCCCTACAC TGCAGCAGTC |
| 2401 | CTAGAATATA GAGTCTCAAT CCATGATTCA GAAGACGCCA AATTCAATGA TATTAACATG |
| 2461 | GCCAACGGTA ACGGTTACAC CGTTTTTGAC GCACAACATC CATACAAAGC TAATGTTGCT |
| 2521 | GTTAAAAGGG AACTTCACAC CCCAGAAAGT GACAGGTCAT GTATACATTT GGAATTTGAT |
| 2581 | ATCGCTGGTA GTGGTTTGAC TTACGAAACA GGTGACCATG TCGGAGTACT TTGCGATAAT |
| 2641 | TTGTCAGAAA CTGTTGATGA AGCTTTGAGG TTATTGGATA TGTCACCAGA TACTTACTTC |
| 2701 | TCATTGCATG CAGAAAAAGA AGACGGAACT CCAATATCAA GCTCGCTTCC CCCTCCATTC |
| 2761 | CCTCCCTGTA ACTTAAGAAC AGCCCTAACT AGATATGCTT GTTTACTGTC TTCTCCAAAG |
| 2821 | AAAAGTGCTT TGGTTGCATT GGCAGCCCAC GCATCCGATC CTACCGAAGC TGAGAGATTA |
| 2881 | AAGCATTTGG CTTCACCAGC CGGTAAAGAT GAATACAGTA AGTGGGTAGT GGAGAGCCAA |
| 2941 | AGATCGCTTT TAGAAGTGAT GGCTGAGTTT CCAAGTGCTA AACCTCCTCT GGGTGTATTT |
| 3001 | TTCGCTGGTG TGGCCCCAAG ATTGCAGCCT AGATTTTATT CCATATCCTC ATCTCCAAAA |
| 3061 | ATTGCCGAAA CCAGAATTCA CGTGACATGT GCTCTGGTCT ACGAAAAGAT GCCAACAGGT |
| 3121 | AGGATTCACA AGGGTGTCTG TTCTACCTGG ATGAAAAATG CTGTACCCTA TGAAAAATCC |
| 3181 | GAAAATTGTT CTAGTGCACC AATTTTCGTA AGACAATCTA ATTTCAAGTT ACCAAGCGAT |
| 3241 | TCTAAAGTAC CCATTATTAT GATCGGTCCA GGTACTGGTT TGGCCCCATT CAGAGGCTTC |
| 3301 | TTGCAAGAAA GATTGGCTTT AGTGGAGAGT GGAGTTGAAT TGGGTCCTTC AGTTTTATTC |
| 3361 | TTTGGTTGTA GAAACAGAAG AATGGACTTT ATCTACGAAG AAGAATTGCA GAGATTTGTT |
| 3421 | GAAAGTGGTG CATTGGCCGA ATTGAGTGTT GCATTCAGCA GGGAAGGTCC AACCAAAGAA |
| 3481 | TACGTTCAAC ACAAGATGAT GGACAAGGCT TCTGATATCT GGAATATGAT TTCCCAAGGT |
| 3541 | GCTTATTTGT ATGTTTGTGG TGACGCTAAA GGAATGGCTA GAGATGTTCA TAGATCACTG |
| 3601 | CATACAATCG CACAAGAACA AGGTAGCATG GATTCAACAA AAGCAGAGGG CTTTGTAAAG |
| 3661 | AATCTTCAGA CAAGCGGTAG ATATCTGAGA GATGTATGGT AA |
| // | |

LOCUS At4CL-2 1671 bp

| | |
|---|---|
| 1 | ATGACGACAC AAGATGTGAT AGTCAATGAT CAGAATGATC AGAAACAGTG TAGTAATGAC |
| 61 | GTCATTTTCC GATCGAGATT GCCTGATATA TACATCCCTA ACCACCTCCC ACTCCACGAC |
| 121 | TACATCTTCG AAAATATCTC AGAGTTCGCC GCTAAGCCAT GCTTGATCAA CGGTCCCACC |
| 181 | GGCGAAGTAT ACACCTACGC CGATGTCCAC GTAACATCTC GGAAACTCGC CGCCGGTCTT |
| 241 | CATAACCTCG GCGTGAAGCA ACACGACGTT GTAATGATCC TCCTCCCGAA CTCTCCTGAA |
| 301 | GTAGTCCTCA CTTTCCTTGC CGCCTCCTTC ATCGGCGCAA TCACCACCTC CGCGAACCCG |
| 361 | TTCTTCACTC CGGCGGAGAT TTCTAAACAA GCCAAAGCCT CCGCTGCGAA ACTCATCGTC |
| 421 | ACTCAATCCC GTTACGTCGA TAAAATCAAG AACCTCCAAA ACGACGGCGT TTTGATCGTC |
| 481 | ACCACCGACT CCGACGCCAT CCCCGAAAAC TGCCTCCGTT TCTCCGAGTT AACTCAGTCC |
| 541 | GAAGAACCAC GAGTGGACTC AATACCGGAG AAGATTTCGC CAGAAGACGT CGTGGCGCTT |
| 601 | CCTTTCTCAT CCGGCACGAC GGGTCTCCCC AAAGGAGTGA TGCTAACACA CAAAGGTCTA |
| 661 | GTCACGAGCG TGGCGCAGCA AGTCGACGGC GAGAATCCGA ATCTTTACTT CAACAGAGAC |
| 721 | GACGTGATCC TCTGTGTCTT GCCTATGTTC CATATATACG CTCTCAACTC CATCATGCTC |
| 781 | TGTAGTCTCA GAGTTGGTGC CACGATCTTG ATAATGCCTA AGTTCGAAAT CACTCTCTTG |
| 841 | TTAGAGCAGA TACAAAGGTG TAAAGTCACG GTGGCTATGG TCGTGCCACC GATCGTTTTA |
| 901 | GCTATCGCGA AGTCGCCGGA GACGGAGAAG TATGATCTGA GCTCGGTTAG GATGGTTAAG |
| 961 | TCTGGAGCAG CTCCTCTTGG TAAGGAGCTT GAAGATGCTA TTAGTGCTAA GTTTCCTAAC |
| 1021 | GCCAAGCTAG GTCAGGGCTA TGGGATGACA GAAGCAGGTC CGGTGCTAGC AATGTCGTTA |
| 1081 | GGGTTTGCTA AAGAGCCGTT TCCAGTGAAG TCAGGAGCAT GTGGTACGGT GGTGAGGAAC |
| 1141 | GCCGAGATGA AGATACTTGA TCCAGACACA GGAGATTCTT TGCCTAGGAA CAAACCCGGC |
| 1201 | GAAATATGCA TCCGTGGCAA CCAAATCATG AAAGGCTATC TCAATGACCC CTTGGCCACG |
| 1261 | GCATCGACGA TCGATAAAGA TGGTTGGCTT CACACTGGAG ACGTCGGATT TATCGATGAT |
| 1321 | GACGACGAGC TTTTCATTGT GGATAGATTG AAAGAACTCA TCAAGTACAA AGGATTTCAA |
| 1381 | GTGGCTCCAG CTGAGCTAGA GTCTCTCCTC ATAGGTCATC CAGAAATCAA TGATGTTGCT |
| 1441 | GTCGTCGCCA TGAAGGAAGA AGATGCTGGT GAGGTTCCTG TTGCGTTTGT GGTGAGATCG |
| 1501 | AAAGATTCAA ATATATCCGA AGATGAAATC AAGCAATTCG TGTCAAAACA GGTTGTGTTT |
| 1561 | TATAAGAGAA TCAACAAAGT GTTCTTCACT GACTCTATTC CTAAAGCTCC ATCAGGGAAG |
| 1621 | ATATTGAGGA AGGATCTAAG AGCAAGACTA GCAAATGGAT TAATGAACTA G |
| // | |

LOCUS HaCHS 1173 bp

| | |
|---|---|
| 1 | ATGGTTACTG TTGAAGAAGT TAGAAAAGCT CAAAGGGCAG AAGGTCCAGC CACAGTGATG |
| 61 | GCTATTGGAA CCGCAGTTCC TCCAAATTGT GTAGATCAGG CCACTTATCC TGACTACTAC |
| 121 | TTTAGAATAA CAAACTCTGA GCATAAGGCT GAATTGAAAG AAAAGTTCCA AAGGATGTGC |
| 181 | GACAAATCAC AGATCAAGAA AAGATACATG TACCTTAATG AGGAAGTCCT AAAGGAAAAC |
| 241 | CCAAATATGT GTGCATACAT GGCCCCTTCC CTTGACGCTA GACAAGATAT TGTGGTTGTA |
| 301 | GAGGTCCCAA AATTGGGCAA GGAAGCAGCT GTTAAAGCCA TAAAGGAATG GGGTCAACCT |
| 361 | AAGAGCAAAA TCACCCACCT TGTGTTTTGC ACTACAAGCG GAGTTGACAT GCCAGGCGCA |
| 421 | GATTATCAGC TAACCAAACT TTTGGGTTTA AGGCCTTCTG TAAAAAGATT GATGATGTAC |
| 481 | CAACAAGGTT GTTTCGCTGG AGGCACTGTC TTAAGACTAG CCAAGGATCT TGCAGAGAAC |
| 541 | AACAAAGGTG CTAGGGTGTT GGTTGTATGC TCAGAAATTA CAGCCGTCAC CTTTAGAGGA |
| 601 | CCAACTGACA CTCACTTAGA TTCCCTAGTT GGTCAGGCAT TGTTTGGCGA CGGTGCTGCC |
| 661 | GCAATAATCA TTGGAAGTGA TCCTATTCCA GAGGTGGAAA AGCCTCTTTT TGAACTTGTT |
| 721 | AGCGCTGCCC AAACTATATT GCCAGATTCT GAGGGTGCAA TCGACGGCCA CTTAAGGGAA |
| 781 | GTAGGTCTAA CCTTCCATCT TTTGAAAGAT GTCCCTGGTT TAATTTCAAA GAACGTGGAA |
| 841 | AAATCCCTAA CAGAGGCTTT TAAACCATTG GGTATAAGTG ACTGGAATAG CTTATTCTGG |
| 901 | ATCGCTCACC CAGGCGGCCC TGCCATACTT GACCAGGTTG AAGCAAAATT GAGCTTAAAG |
| 961 | CCAGAAAAAC TAAGAGCTAC TAGACATGTA TTGTCAGAGT ATGGTAACAT GTCCAGTGCC |
| 1021 | TGTGTCCTTT TCATTTTGGA TGAAATGAGG AGAAAAAGCA AGGAGGACGG CCTAAAAACC |
| 1081 | ACAGGTGAGG GAATCGAATG GGGTGTTCTA TTCGGCTTTG GTCCAGGCCT TACTGTGGAG |
| 1141 | ACAGTTGTAC TTCATTCAGT CGCAATTAAT TAG |
| // | |

LOCUS PhCHI 726 bp

| | |
|---|---|
| 1 | ATGTCTCCAC CAGTTTCTGT TACAAAAATG CAAGTCGAAA ATTATGCTTT TGCACCAACA |
| 61 | GTGAACCCTG CCGGTTCCAC CAATACTTTG TTCTTAGCTG GAGCAGGCCA TAGAGGTCTA |
| 121 | GAGATTGAAG GAAAGTTTGT GAAATTCACA GCCATAGGCG TATACCTTGA GGAAAGTGCT |
| 181 | ATCCCATTTT TGGCAGAAAA GTGGAAAGGT AAGACCCCTC AGGAGTTAAC TGATAGCGTC |
| 241 | GAGTTCTTTA ATGGGGTGGT TACAGGTCCA TTCGAAAAGT TTACCAGAGT AACTATGATT |
| 301 | CTACCTCTTA CAGGAAAGCA ATATTCTGAG AAAGTCGCCG AAAACTGTGT TGCTCACTGG |
| 361 | AAGGGCATAG GTACCTACAC TGATGACGAA GGAAGGGCAA TCGAGAAATT CTTGGATGTG |
| 421 | TTTAGATCAG AAACATTCCC ACCTGGTGCT TCCATTATGT TTACTCAGAG TCCATTAGGC |
| 481 | TTGTTAACCA TCAGCTTTGC CAAGGACGAT TCAGTTACCG GTACTGCAAA TGCTGTAATC |
| 541 | GAGAACAAAC AACTATCAGA AGCCGTCCTT GAATCCATTA TTGGAAAGCA TGGTGTGAGT |
| 601 | CCTGCAGCCA AATGCTCTGT TGCCGAGAGA GTAGCAGAAT TGTTAAAAAA GAGCTATGCT |
| 661 | GAAGAGGCCT CAGTGTTCGG CAAACCAGAA ACCGAAAAGT CCACAATACC TGTTATCGGT |
| 721 | GTGTAG |
| // | |

TABLE 5
List of oligonucleotides

| CCM pathway | | |
|---|---|---|
| genes and | | |
| promoters | | |
| Primer name | Primer sequence, 5′ to 3′ | |
| ID1564_PTEF1- | CGTGCGAU | Forward primer for USER cloning of the |
| >_U2_fw | GCACACACCATAGCTTC | TEF1 promoter |
| ID1565_PTEF1- | ATGACAGAU | Reverse primer for USER cloning of the |
| >_U2_rv | TTGTAATTAAAACTTAG | TEF1 promoter |
| ID3108_PTEF1_ | AGCTACTGAU | Forward primer for USER cloning of the |
| for_fusion_fw | GCACACACCATAGCTTC | TEF1 promoter fused with the TDH3 |
| | | promoter for bidirectional expression |
| ID3107_PTDH3_ | ATCAGTAGCU | Forward primer for USER cloning of the |
| for_fusion_fw | ATAAAAAACACGCTTTTTCAG | TDH3 promoter fused with the TEF1 |
| | | promoter for bidirectional expression |
| ID1853_PTDH3 | ACCTGCACU | Reverse primer for USER cloning of the |
| <-_U1_rv | TTTGTTTGTTTATGTGTGTTTATT | TDH3 promoter |
| | C | |
| ID3097_CaCatA_ | ATCTGTCAU | Forward primer for USER cloning |
| U2_fw | AAAACAATGTCCCAAG | of CaCatA |
| ID3098_CaCatA_ | CACGCGAU | Reverse primer for USER cloning |
| U2_rv | TTACAACTTGATTTCAGC | of CaCatA |
| ID3103_KpAroY. | ATCTGTCAU | Forward primer for USER cloning |
| B_U1_fw | AAAACAATGATCTGTCC | of KpAroY.B |
| ID3104_KpAroY. | CGTGCGAU | Reverse primer for USER cloning |
| B_U1_rv | TCATTCAATTTCTTGAGC | of KpAroY.B |
| ID3105_KpAroY. | ATCTGTCAU | Forward primer for USER cloning |
| Ciso_U2_fw | AAAACAATGACCGCCCCAATC | of KpAroY.Ciso |
| ID3016_KpAroY. | CACGCGAU | Reverse primer for USER cloning of |
| Ciso_U2_rv | TTACTTAGCGGAACCTTGATTC | KpAroY.Ciso |
| ID3095_KpAroY. | ATCTGTCAU | Forward primer for USER cloning |
| D_U2_fw | AAAACAATGATCTGTCC | of KpAroY.D |
| ID3096_KpAroY. | CACGCGAU | Reverse primer for USER cloning of |
| D_U2_rv | TTATCTCTTATCTTCTGG | KpAroY.D |
| ID3101_PaAroZ_ | AGTGCAGGU | Forward primer for USER cloning |
| U1_fw | AAAACAATGCCATCCAAG | of PaAroZ |
| ID3102_PaAroZ_ | CGTGCGAU | Reverse primer for USER cloning of |
| U1_rv | TCACAAAGCAGCTGACAAAG | PaAroZ |
| ID1391_ScTkl1_ | AGTGCAGGU | Forward primer for USER cloning of Tkl1 |
| U1_fw | AAAACAATGACTCAATTCACTGA | |
| | CATTG | |
| ID1392_ScTkl1_ | CGTGCGAU | Reverse primer for USER cloning of Tkl1 |
| U1_rv | TCAGAAAGCTTTTTTCAAAGGAG | |
| LTTR sensor and reporter promoters | | |
| Primer name | Primer sequence, 5′ to 3′ | |
| MeLS069-F | GATGAATGCGGCCGCTTTA | Forward primer for random mutagenesis |
| | | of BenM-EBD |
| MeLS093-R | CAATACGCCATCAAGTTGCTAAG | Reverse primer for random mutagenesis |
| | C | of BenM-EBD |
| MeLS071-F | CTCCTTCCTTTTCGGTTAGAGCG | Tailed primer for BenM-EBD library |
| | GATGAATGCGGCCGCTTTA | assembly by gap repair |
| MeLS094-R | TCATTTCTTTTACCAATACGCCAT | Tailed primer for BenM-EBD library |
| | CAAGTTGCTAAGC | assembly by gap repair |
| MeLS001_F | ATCTGTCAUAAAACAATGGAATT | Forward primer for USER cloning of BenM |
| | GAGACAC | |
| MeLS003_R | CACGCGAUTTACCAATTTGGTGG | Reverse primer for USER cloning of BenM |
| | TTCAG | |
| MeLS005_F | CGTGCGAUATACTCCATAGGTAT | Forward primer for USER cloning of the |
| | TTT | BenM binding site |
| MeLS008_R | CACGCGAUTTATTTGTACAATTC | Reverse primer for USER cloning of the |
| | ATCCA | yeGFP ORF |
| MeLS009_F | ATCTGTCAUAAAACAATGTCTAA | Forward primer for USER cloning of the |
| | AGGTG | yeGFP ORF |
| MeLS0046_F | CGTGCGAUTTCTTAGGCACAACA | Forward primer for USER cloning of the |
| | ATATTTATAAAAGAAG | REV1 promoter |
| MeLS0047_R | ATGACAGAUCGCTGGATATGCC | Reverse primer for USER cloning of the |
| | TAGAAATGC | REV1 promoter |
| MeLS0048_F | CGTGCGAUGGAAAACCAAGAAA | Forward primer for USER cloning of the |
| | TGAATTATATTTCC | 491 bp CYC1 promoter |
| MeLS0049_R | ATGACAGAUTATTAATTTAGTGT | Reverse primer for USER cloning of the |
| | GTGTATTTGTGTTTGTG | CYC1 promoter |
| MeLS0052_F | CGTGCGAUCCAGGCAACTTTAG | Forward primer for USER cloning of the |
| | TGCTGACAC | 209 bp CYC1 promoter |
| MeLS0056_F | CGTGCGAUATAAAAAACACGCTT | Forward primer for USER cloning of the |
| | TTTCAGTTCG | TDH3 promoter |
| MeLS0057_R | ATGACAGAUTTTGTTTGTTTATG | Reverse primer for USER cloning of the |
| | TGTGTTTATTCGA | TDH3 promoter |
| MeLS0062_F | CGTGCGAUGAAAGACCACACCC | Forward primer for USER cloning of the |
| | ACGCG | RNR2 promoter |
| MeLS0063_R | ATGACAGAUGGTAATTGGACAA | Reverse primer for USER cloning of the |
| | ATAAATACGTGTATTAAG | RNR2 promoter |
| MeLS0064_F | CGTGCGAUTCAAGCCCACGCGT | Forward primer for USER cloning of the |
| | AGGC | 272 bp CYC1 promoter |
| MeLS0074_F | CGTGCGAUTCGAGCAGATCCGC | Forward primer for USER cloning of the |
| | CAGG | 249 bp CYC1 promoter |
| MelS101-R | CACGCGAUTCAGCAGATGCCTG | Reverse primer for USER cloning of FdeR |
| | GCAGC | |
| MelS108-F | ATCTGTCAUAAAACAATGCGTTT | Forward primer for USER cloning of FdeR |
| | CAACAAGCTCGAC | |
| TISNO-53F | ATCTGTCAUAAAACAATGATTGA | Forward primer for USER cloning of PcaQ |
| | TGCACGT | |
| TISNO-54R | CACGCGAUTTATGCTGTTCTTTT | Reverse primer for USER cloning of PcaQ |
| | GGCTTC | |
| TISNO-57F | ATCTGTCAUAAAACAATGAAACG | Forward primer for USER cloning of ArgP |
| | TCCTGA | |
| TISNO-58R | CACGCGAUTCAATCTTGTCTTAA | Reverse primer for USER cloning of ArgP |
| | CACCTTATG | |
| TISNO-59F | ATCTGTCAUAAAACAATGAAGGA | Forward primer for USER cloning of MdcR |
| | CGACAT | |
| TISNO-60R | CACGCGAUTTACCTGTTAATAGA | Reverse primer for USER cloning of MdcR |
| | ACGAGCATA | |
| Genotyping | | |
| primers | | |
| Primer name | Primer sequence, 5′ to 3′ | |
| ID893 XII-2- | CGAAGAAGGCCTGCAATTC | Genotyping of genomic integration locus |
| up-out-sq | | |
| ID894 XII-2- | GGCCCTGATAAGGTTGTTG | Genotyping of genomic integration locus |
| down-out-sq | | |
| ID897 XII-4- | GAACTGACGTCGAAGGCTCT | Genotyping of genomic integration locus |
| out-seq_fw | | |
| ID898 XII-4- | CGTGAAATCTCTTTGCGGTAG | Genotyping of genomic integration locus |
| down-out-sq | | |
| ID903_X-3- | TGACGAATCGTTAGGCACAG | Genotyping of genomic integration locus |
| out-seq_fw | | |
| ID904_X-3- | CCGTGCAATACCAAAATCG | Genotyping of genomic integration locus |
| down-out-sq | | |
| ID905_X-4- | CTCACAAAGGGACGAATCCT | Genotyping of genomic integration locus |
| out-seq_fw | | |
| ID906_X-4- | GACGGTACGTTGACCAGAG | Genotyping of genomic integration locus |
| down-out-sq | | |
| ID907_XI-1- | CTTAATGGGTAGTGCTTGACACG | Genotyping of genomic integration locus |
| out-seq_fw | | |
| ID908_XI-1- | GAAGACCCATGGTTCCAAGGA | Genotyping of genomic integration locus |
| down-out-sq | | |
| ID911_XI-3- | GTGCTTGATTTGCGTCATTC | Genotyping of genomic integration locus |
| out-seq_fw | | |
| ID2220 | CCTGCAGGACTAGTGCTGAG | Genotyping of genomic integration locus |
| Sc_ColoPCR_fw | | |
| ID2221 | GTTGACACTTCTAAATAAGCGAA | Genotyping of genomic integration locus |
| Sc_ColoPCR_rv | TTTC | |
| MeLS0082_F | AAAAATAAATAGGGACCTAGACT | Sequencing primer located in the CYC1 |
| | TCAGG | terminator |
| MeLS0053_R | CTGCAGGAATTCGATATCAAGC | Reverse sequencing primer located in the |
| | | KI.URA terminator |
| MeLS0054_F | TCAATTGAGATGAGCTTAATCAT | Forward sequencing primer located in the |
| | GTC | KI.URA promoter |
| MeLS0055_R | ATTATTACAGTCACTCAGACAGA | Sequencing primer located in the XII-1 |
| | GCAC | down homology region |
| MeLS0058_F | GTGAAGTGATCATGCACATCGC | Sequencing primer located in the REV1 |
| | | promoter |
| MeLS0083_R | GAGGTTCCAGACCAGTTAAGACT | Sequencing primer located in BenM DNA- |
| | ACTC | binding domain |
| TISNO-15F | GTAAGCCAGATTAAAATTCACG | Sequencing primer located in REV1 |
| | | promoter |
| TISNO-62R | TAGCATCACCTTCACCTTCACC | Genotyping of genomic integration locus |
| | | (anneals to yeGFP) |
| TISNO-63F | CCTGAAATTATTCCCCTACTTGA | Sequencing primer located in TDH3 |
| | C | promoter |
| TISNO-65R | TATCGGATAACAACACCGCTG | Genotyping of genomic integration locus |
| | | (anneals to REV1p) |
| TISNO-66R | CTGTTCACCCAGACACCTAC | Genotyping of genomic integration locus |
| | | (anneals to TDH3p) |
| TISNO-67R | GCGGAGTCCGAGAAAATCTG | Genotyping of genomic integration locus |
| | | (anneals to TEF1p) |
| Res417 R | TCTCAGGTATAGCATGAGGTCGC | Genotyping of genomic integration locus, |
| | TCAT | internal assembler primer |
| Res418 F | CCTGCAGGACTAGTGCTGAGGC | Genotyping of genomic integration locus, |
| | ATTAAT | internal assembler primer |
| RE5395 XI-2 | GTTTGTAGTTGGCGGTGGAG | Genotyping of genomic integration locus |
| UP F | | |
| RE5396 XI-2 | GAGACAAGATGGGGCAAGAC | Genotyping of genomic integration locus |
| DW | | |
| RES511 XVI-20 | GGCTTGTGGTCACCTGTCAT | Genotyping of genomic integration locus |
| UP F | | |
| RE5512 XVI-20 | GAATTATGGTAATTTTGATTATC | Genotyping of genomic integration locus |
| DW R | | |
| RE5658 X-2-UP | TGCGACAGAAGAAAGGGAAG | Genotyping of genomic integration locus |
| F | | |
| RE5659 X-2 | GAGAACGAGAGGACCCAACAT | Genotyping of genomic integration locus |
| DW R | | |
| Naringenin pathway genes and promoters | | |
| Primer name | Primer sequence, 5′ to 3′ | |
| RES194 C4H F | AGCGATACGUAAAATGGATTTGT | Forward primer for USER cloning of the |
| | TATTGCTGGAAAAG | C4H |
| RES195 C4H R | CACGCGAUTTACCATACATCTCT | Reverse primer for USER cloning of the |
| | CAGATATCTAC | C4H |
| RE5196 AtPAL2 | ATCAACGGGUAAAATGGACCAA | Forward primer for USER cloning of the |
| F | ATTGAAGCAATGC | AtPAL2 |
| RE5197 AtPAL2 | CGTGCGAUTTAGCAGATTGGAA | Reverse primer for USER cloning of the |
| R | TAGGTGCAC | AtPAL2 |
| RE5198 At4CL2 | AGCGATACGUAAAATGACGACA | Forward primer for USER cloning of the |
| F | CAAGATGTGATAGTC | 4Cl2 |
| RES199 At4CL2 | CACGCGAUCTAGTTCATTAATCC | Reverse primer for USER cloning of the |
| R | ATTTGCTAG | 4Cl2 |
| RE5569 CHI F | AGCGATACGUAAAATGTCTCCAC | Forward primer for USER cloning of the |
| | CAGTTTCTGTTAC | CHI |
| RE5570 CHI R | CACGCGAUCTACACACCGATAA | Reverse primer for USER cloning of the |
| | CAGGTATTG | CHI |
| RE5571 CHS F | CGTGCGAUTAATTAATTGCGACT | Forward primer for USER cloning of the |
| | GAATGAAG | CHS |
| RE5572 CHS R | ATCAACGGGUAAAATGGTTACTG | Reverse primer for USER cloning of the |
| | TTGAAGAAGTTAG | CHS |
| RE5573 ATR2 | agctgcagcUaaagaagctgcagcaaa | Forward primer for USER cloning of the |
| L5 F | agctTCCAGTAGCTCTTCCTCCTC | ATR2 (includes L5 linker) |
| RES 574 L5 | AgctgcagcUtcttttgctgcagcttcagc | Reverse primer for USER cloning of the |
| C4H R | gctACAATTTCTGGGTTTCATG | C4H (includes L5 linker) |
| RE5407 pTDH3 | ACCCGTTGAUTTTTGTTTGTTTAT | Forward primer for USER cloning of the |
| F | GTGTGTTTATTCG | TDH3 promoter |
| RE5408 pTDH3 | CACGCGAUGATCTCAGTTCGAG | Reverse primer for USER cloning of the |
| R | TTTATCATTATCA | TDH3 promoter |
| RE5454 pPGK1 | ACCCGTTGAUGCCGCTTGTTTTA | Reverse primer for USER cloning of the |
| R | TATTTGTTGTAAAAAG | PGK1 promoter |
| RE5455 pPGK1 | CACGCGAUGGCCTGGAAGTACC | Forward primer for USER cloning of the |
| F | TTCAAAGAATG | PGK1 promoter |
| RE5456 pTEF1 | CGTGCGAUGCCGCACACACCAT | Forward primer for USER cloning of the |
| F | AGCTTCAAAATG | TEF1 promoter |
| RE5457 pTEF1 | ACGTATCGCUGTGAGTCGTATTA | Reverse primer for USER cloning of the |
| R | CGGATCC | TEF1 promoter |
| RE5568 pPDC1 | CGTGCGAUGCCGATCTATGCGA | Forward primer for USER cloning of the |
| F | CTGGGTGAG | PDC1 promoter |
| RE5640 pPDC1 | ACGTATCGCUTTTTGATAGATTT | Reverse primer for USER cloning of the |
| R | GACTGTGTTATTTTGCG | PDC1 promoter |
| RE5460 | ACCCGTTGAUTTTTGTTTGTTTAT | Reverse primer for USER cloning of the |
| pTDH3/pTEF2 | GTGTG | bidirectional promoter |
| R | | |
| RE5461 pTDH3 | ACGTATCGCUTGTTTAGTTAATT | Forward primer for USER cloning of the |
| /pTEF2 F | ATAGTTC | bidirectional promoter |

TABLE 6

| | | | |
|---|---|---|---|
| AlsR | Activator (local) | Bacillus subtilis | Renna et al. (1993) Bartowsky |
| AmpR | Activator (local) | Rhodobacter | & Normark (1993) |
| | Activator (local) | capsulatus | |
| ArgP | Activator (global) | Enterobacter cloacae | Nandineni & Gowrishankar |
| BenM | Activator (local) | Citrobacter freundii | (2004) Collier et al. (1998) |
| BlaA | Activator (global) | Escherichia | Raskin et al. (2003) |
| CatM | Activator (global) | Acinetobacter spp. | Chugani et al. (1998) van |
| CbbR | Activator (global) | Streptomyces spp. | Keulen et al. (2003) |
| CfxR | | Acinetobacter | Windho \ddot{ }vel 991) |
| | | calcolaceticus | |
| | | Pseudomonas putida | |
| | | Xanthobacter flavus | |
| ChiR | Activator (local) | Serratia marcescens | Suzuki et al. (2001) |
| CidR | Activator (local) | Staphylococcus Spp. | Yang et al. (2005) |
| | | Bacillus anthracis | Ahn et al. (2006) |
| ClcR | Activator (local) | Pseudomonas putida | Coco et al. (1993) |
| CrqA | Activator/Repressor | Neisseria meningitidis | Deghmane et al. (2000) |
| | (global) | | |
| CynR | Activator (local) | Escherichia coli | Sung & Fuchs (1992) |
| CysB | Activator (global) | Salmonella enterica | van der Ploeg et al. (1997) |
| | | Typhimurium | |
| | | Escherichia coli | |
| CysL | Activator (global) | Bacillus subtilis | Guillouard et al. (2002) |
| GltC | Activator (local) | Bacillus subtilis | Picossi et al. (2007) |
| HupR | Activator (global) | Vibrio vulnificus | Litwin & Quackenbush (2001) |
| HvrB | Activator (global) | Rhodobacter | Buggy et al. (1994) |
| IlvR | Activator (local) | Caulobacter | Malakooti & Ely (1994) |
| IlvY | Activator (local) | Escherichia coli | Wek & Hatfield (1988) |
| IrgB | Activator (local) | Vibrio cholerae | Goldberg et al. (1991) |
| LeuO | Activator/Repressor | Salmonella enterica | Herna\acute{ }ndez-Lucas et al. (2008) |
| | (global) | Typhimurium | |
| LrhA | Activator (global) | Escherichia coli | Lehnen et al. (2002) |
| LysR | Activator (local) | Escherichia coli | Stragier et al. (1983) |
| MdcR | Activator (local) | Klebsiella pneumoniae | Peng et al. (1999) |
| MetR | Activator (global) | Streptococcus spp. | Kovaleva & Gelfand (2007) |
| MleR | Activator (local) | Lactococcus lactis | Renault et al. (1989) |
| MtaR | Activator (global) | Group B streptococci | Shelver et al. (2003) |
| MvfR | Activator (global) | Pseudomonas | Cao et al. (2001) |
| | | aeruainosa | |
| NagR | Activator (local) | Ralstonia eutropha | Jones et al. (2003) |
| NahR | Activator (local) | NAH7 plasmid of | Park et al. (2002) |
| NhaR | Activator (local) | Escherichia coli | Dover & Padan (2001) |
| NocR | Activator (local) | Ti plasmids of | von Lintig et al. (1994) |
| | | Agrobacterium | |
| | | [symbol] | |

[symbol]

indicates data missing or illegible when filed

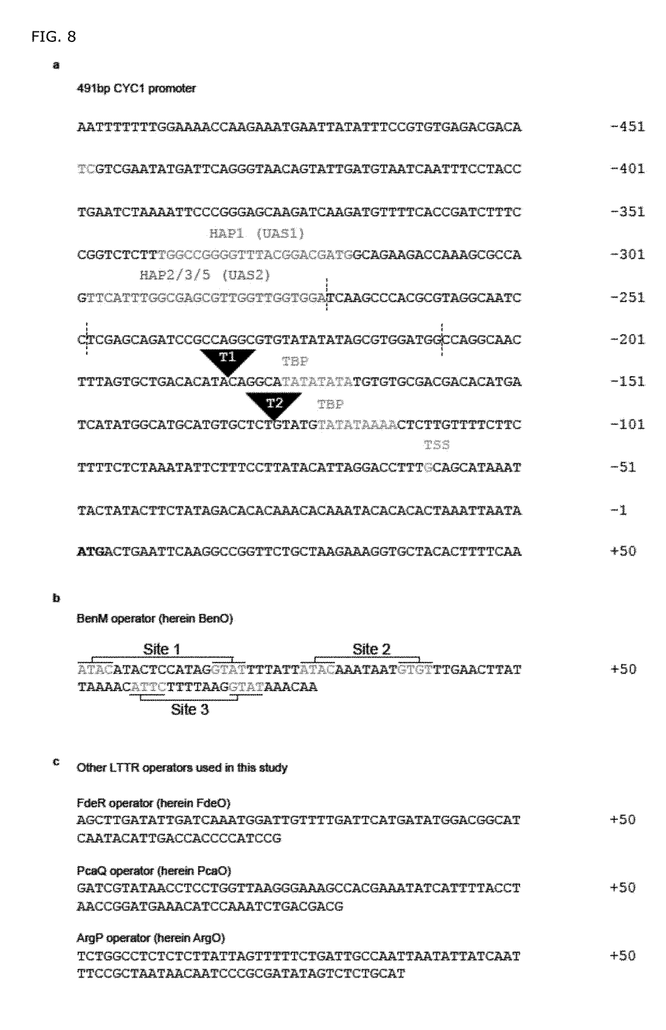

TABLE 7

| | Specific | | | |
|---|---|---|---|---|
| Super | ligand | Transcrip | Operator | |
| family | activator | tional | sequence | reference |
| AraC/ | p- | pobR | GCCGGCGC | http://www.pseudomonas.com/feature/ |
| XylS | hydroxy- | | ATGCGCCG | intergenic?start=280759&stop= |
| | benzoate | | CCGGCCAG | 280935&repliconid=136&src=map |
| | | | CCATAA | |
| LuxR | N-(3-oxodo- | LasR | CTATGTCTT | http://www.pseudomonas.com/feature/ |
| | decanoyl) | | TTGTTAG | intergenic?start=1077905&stop= |
| | homoserine | | | 1078461&repliconid=136&src=map |
| | lactone (3- | | | |
| | oxo-C12- | | | |
| | HSL) and N- | | | |
| | butyryl | | | |
| | homoserine | | | |
| | lactone | | | |
| LTTR | multiple | mvfR | TTCGGACTC | http://www.pseudomonas.com/feature/ |
| | quorum | | CGAA | intergenic?start=1077905&stop= |
| | sensing | | | 1078461&repliconid=136&src=map + |
| | | | | Xiao et al 2006 |
| LTTR | flavonoids, | nodD | AGATTAGTA | yang et al 2012; |
| | naringenin, | | AAATTGATT | http://www.ncbi.nlm.nih.gov/gene/ |
| | hesperetin | | GTTGGGAT | 4403938 |
| | | | AGCTATCAT | |
| | | | CCACGATAT | |
| | | | GGATG | |
| | benzoate | benR | CCGAAAAA | putativ, COWLESet al 2000, |
| | | | GTACCGAA | http://www.ncbi.nlm.nih.gov/gene/ |
| | | | CATCCGTAA | 1046807 |
| | | | ATCTGGATA | |
| | | | ACGTTCTGC | |
| | | | ACAATCCG | |
| | | | GATAGCCC | |
| | | | CCCGCCAG | |
| | | | CCGTCTCCC | |
| | | | TAAC | |
| Lrp/ | binding | lysM | TAAAATCGT | Brinkman et al |
| AsnC | inhibition | | ACCACTTAT | 2002+http://www.ncbi.nlm.nih.gov/ |
| | with lysine | | TACTAAAAA | gene/1453332 |
| | | | CTTTTTCTA | |
| | | | CACAAAACT | |
| | | | AAGTTAGTA | |
| | | | TCTAAC | |
| LTTR | sulfur | cysB | TGTTGAAAT | Hryniewicz et al, 1991; |
| | sources? (no | | TAAAGGCCT | Delic-Atree 1997 + |
| | cysteine) N- | | TTAGAAACT | http://www.pseudomonas.com/feature/ |
| | acetyl-serine | | TGAATTCTA | show/?id=109899&view=sequence |
| | | | TGGACCGA | |
| | | | ACTAAAA | |
| LTTR | sulfur | cysBH | | |
| | sources? (no | | | |
| | cysteine) N- | | | |
| | acetyl-serine | | | |
| LTTR | muconic acid | benM | See FIG. 8 | |
| LTTR | naringenin | FdeR | See FIG. 8 | Siedler S et al., 2014 |
| LTTR | salicylate | NagR | | Jones et al., 2003 |
| LTTR | salicylate | NahR | | Cebolla et al, 1997; van Sint Fiet et |
| | | | | al, 2006; Calcagno review |
| LTTR | protcatechuic | PcaQ | See FIG. 8 | MacLean et al, 2008 |
| | acid | | | |
| LTTR | acetate | AlsR | | Fradrich et al, 2013, de Oliveira et |
| | | | | al, 2013 |
| LTTR | L-arginin | ArgP | See FIG. 8 | Zhou et al, 2010; Laisram et al., |
| | | | | 1997 |
| LTTR | malonate | MdcR | See FIG. 8 | |
| LTTR | | AphB | AACAACCTA | Kovacikova et 2010, Bina et al |
| | | | AGTTTGCA | 2015 |
| | ROS, | soxS | TTTGCATAG | Gil et al |
| | superoxide, | | CGTGAATAT | 2009+http://www.ncbi.nlm.nih.gov/ |
| | paraquate | | GTCAAAATT | gene/1253251 |
| | | | GAT | |
| LTTR | myricetin and | kaeR | CGATTTGC | Pande et al 2001; |
| | kaempferol | | CATTAATC | http://www.ncbi.nlm.nih.gov/gene/ |
| | | | CCATTAGG | ?term=kaeR |
| | | | ACTTTCGT | |
| | | | ATCGGAGA | |
| | | | AGCCTTCAA | |
| | | | CGTTATTAA | |
| | | | ACATCATTG | |
| | | | CTGGACCTT | |
| | | | CTTGCGTCG | |
| | | | GCCGTTTTA | |
| | | | CCGTCCCTC | |
| | | | CAGCACCAA | |
| | | | TATAGCGGT | |
| | | | AAACACCAG | |
| | | | CCAATTCAG | |
| | | | CATTTGGAT | |
| | | | TCACAGCTA | |
| | | | CGTTCGTCT | |
| | | | CATGGTACT | |
| | | | GGTTGGCA | |
| | | | TGGGTTTTT | |
| | | | AGCTCGGC | |
| | | | CAATACTTT | |
| | | | TCGTAAATC | |
| | | | ATAAGGATC | |
| | | | ATTTACCAT | |
| | | | CAGATTACC | |
| | | | TCCTATAAG | |
| | | | TTGCTTACA | |
| | | | ATCACCACT | |
| | | | TTAAGGCAT | |
| | | | AAAATCGTT | |
| | | | GCAAACAAC | |
| | | | TCAACTTTC | |
| | | | GACTAATG | |
| | | | TTATGCCT | |
| | | | AAATGGAA | |
| | | | TAATAAGA | |
| | | | AGAAGGTT | |
| | | | CTTCAAT | |
| | | | (5′)* | |
| | L-arabinose | araC | TCAGGCAG | http://www.ecogene.org/gene/EG1 |
| | | | GATCCGCTA | 0054, |
| | | | ATCTTATGG | http://www.ncbi.nlm.nih.gov/gene/ |
| | | | ACAAAAATG | 1251622 |
| | | | CTAATGCTT | |
| | | | TGCAAAGT | |
| | | | GTGACGCT | |
| | | | GTGCAAATA | |
| | | | TTCAATGTG | |
| | | | GACATTCCA | |
| | | | GCCATAGTT | |
| | | | ATAGACACT | |
| | | | TCTGTTACT | |
| | | | TAATTTTAT | |
| | | | CGCCTGAA | |
| | | | CTGTACGCT | |
| | | | TTTGTTACA | |
| | | | AAGCGCTTT | |
| | | | TCACAAGC | |
| | | | GGGGTTGA | |
| | | | TACGTGCTT | |
| | | | TCATCAAGC | |
| | | | GCAAAGTCT | |
| | | | TGCGGAGA | |
| | | | CGGAAGCT | |
| | | | CTGTCGTCC | |
| | | | TGGTCGATA | |
| | | | TGGACAATT | |
| | | | TGTTTC | |

*Bold sequences are DNA sites where KaeR bind. The site is palindromic meaning that it will bind to two somewhat complementary motifs (the two bold sequences). The sequence in between indicates space which is not necessarily needed to be matching this code in length nor sequence content.

### Results

### Onboarding a Prokaryote Transcription Activator to Yeast

To investigate the potential to build orthogonal biosensors using prokaryotic transcriptional activators in a eukaryotic chassis, we initially selected BenM from Acinetobacter sp ADP1 for several reasons. First, it belongs to the LTTR family, which is one of the most abundant families of transcriptional regulators found in a diverse range of prokaryotes. Second, in Acinetobacter sp ADP1, BenM serves as a native CCM-inducible transcriptional activator (Results, FIG. 1). CCM is an intermediate from aromatic compound catabolism and an important precursor for bioplastics. Moreover CCM biosynthesis was recently refactored in yeast, yet without any high-throughput screening option available. Third, BenM has a well-characterized DNA binding site (herein termed BenO) and mode-of-action (FIG. 1a and FIG. 1). Finally, this protein does not require any binding to regulatory subunits apart from its cognate inducers, which should ensure its orthogonality in non-native chassis.

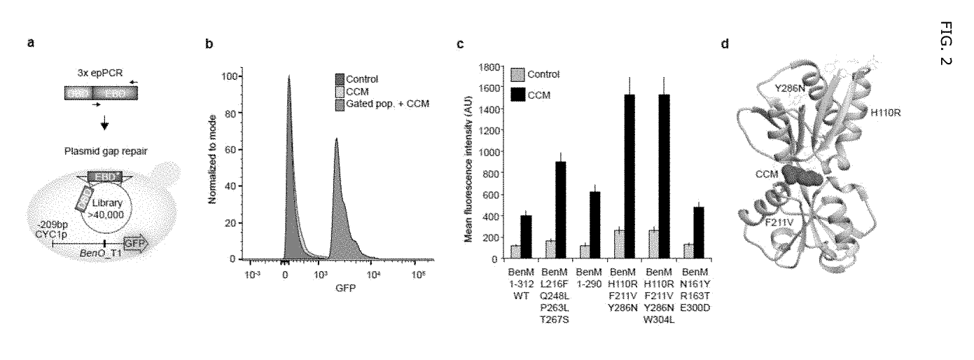

Engineering transcriptional repressors from prokaryotes into eukaryote chassis has emphasized the importance of operator positioning within synthetic eukaryote promoters in relation to transcriptional output. Hence, we first sought to identify optimal positioning of BenO when introduced into a eukaryote promoter. As a first expression cassette the full-length (491 bp) CYC1 promoter (CYC1p) was used to control the expression of green fluorescence protein (GFP) (Olesen, 3., Hahn, S. & Guarente, L. Yeast HAP2 and HAP3 activators both bind to the CYC1 upstream activation site, UAS2, in an interdependent manner. Cell 51, 953-61 (1987)). CYC1p was recently reported as a suitable promoter for introduction of other non-native TF binding sites in yeast, and throughout this study all engineered reporter gene promoters will be based on chromosomally integrated full-length or truncated versions of this promoter. Initially, BenO was introduced into the 491 bp CYC1 promoter immediately upstream of one of the two TATA boxes—TATA-1β (designated 491 bp_CYC1p_BenO_T1) or TATA-2α (designated 491 bp_CYC1p_BenO_T2), or upstream of both (designated 491 bp_CYC1p_BenO_T1/T2)(FIG. 2a). Outputs from these engineered promoters were compared by flow cytometry to expression from the native CYC1p (491 bp_CYC1p) using GFP as the reporter (FIG. 1a and FIG. 2a). In general, introducing BenO negatively impacted the CYC1p activity (FIG. 1b, white columns). However, when co-expressing BenM from the TEF1 promoter we observed 20-fold and 5-fold induction of expression from 491 bp_CYC1p_BenO_T1 and 491 bp_CYC1p_BenO_T1/T2 compared to the promoter activities without co-expression of BenM. For 491 bp_CYC1p_BenO_T2 we observed a modest 30% reduction in expression. Most importantly, BenM did not increase expression of native CYC1p without BenO (FIG. 1b). Taken together these data show that BenM can function as a transcriptional activator in yeast.

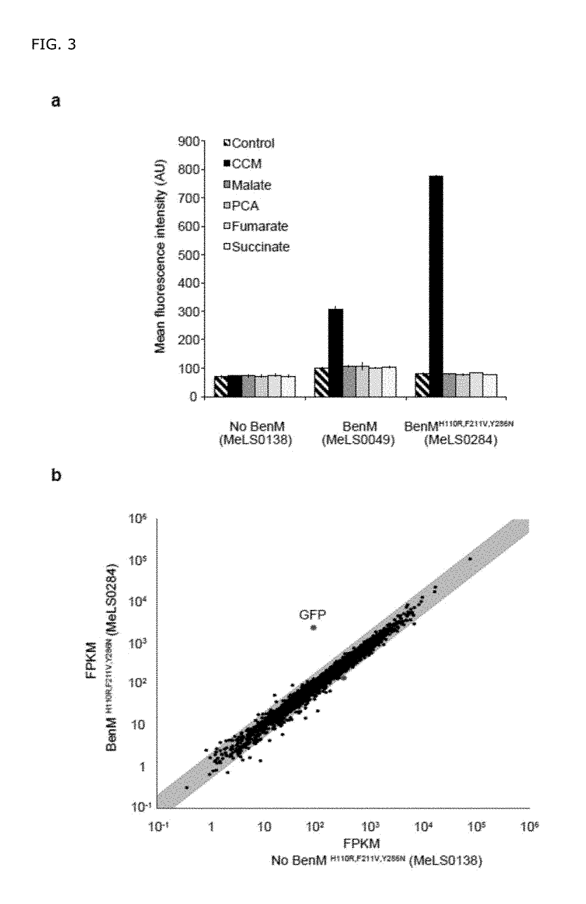

Protonated CCM is directly taken up by yeast at pH 4.5 without any growth defects (FIG. 3a-b). This enables CCM inducibility of the genetic devices to be tested by simple supplement of 200 mg/L CCM to the medium at pH 4.5. Following 24 h of cultivation GFP output was measured using flow cytometry. Here, we observed modest increases (1.3-2.2-fold, FIG. 1b) in reporter output from all CYC1 promoters that harbored BenO, whereas no change was observed from the native CYC1p (FIG. 1b). Also, all engineered promoters showed significant transcriptional activities in the control medium (no CCM) compared to background auto-fluorescence (FIG. 1b).

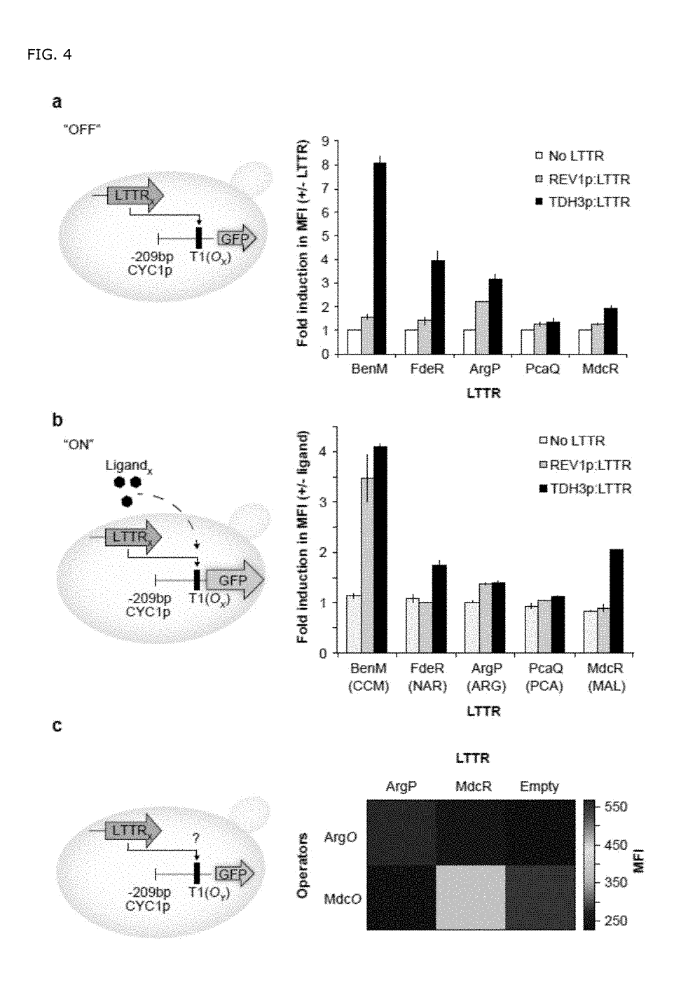

In order to lower the basal activity of the engineered promoters, we removed upstream activating sequences (UAS1 and UAS2) and introduced BenO into truncated versions of the CYC1p (designated 272 bp_CYC1p, 249 bp_CYC1p and 209 bp_CYC1p, FIG. 2a). Also, in order to improve the dynamic range of the genetic device we tuned the production of BenM by placing benM under the transcriptional control of three other native yeast promoters: TDH3p, RNR2p and REV1p. Together with TEF1p, this system allows for an expression range covering almost three orders of magnitude. By combining and chromosomally integrating all possible BenM expression cassettes with all CYC1p-derived reporter constructs, a total of 84 yeast strains were generated, including control strains (FIG. 1c, Table 1 and FIG. 4). Analyzing basal and CCM-induced GFP expression for all strains by flow cytometry we observed reporter outputs that spanned more than two orders of magnitude from the lowest to the highest GFP levels, with most of the high outputs resulting from reporters expressed from full-length CYC1p backbones co-expressed with BenM (FIG. 1c, FIG. 4 and Table 2). Low-expressing strains mostly comprise truncated CYC1p reporter variants without BenO or BenM. These data showed that the BenO_T1 positioning allowed CCM-inducibility of all truncated variants of CYC1p, with the highest dynamic range observed for the minimal promoters 249 bp_CYC1p_BenO_T1 and 209 bp_CYC1p_BenO_T1 (3.2-4.7-fold)(FIG. 1d and Table 2). Among the genetic devices tested, strain MeLS0049 with 209 bp_CYC1p_BenO_T1 controlled by BenM expressed from REV1p showed both low basal activity and high CCM-inducibility (3.8-fold), and was therefore regarded as most suitable for application as a CCM biosensor.

High-Throughput Prototyping of Biosensors Variants

The dynamic range of a biosensor output is an important parameter when evaluating applicability of a biosensor for screening and selection. For this reason, we applied a high-throughput engineering strategy for identifying BenM mutants with higher dynamic ranges when expressed from the weak REV1 promoter. Previous mutagenesis studies identified residues important for ligand-binding in LTTR effector binding domains (EBDs). For this purpose we performed PCR-based mutagenesis of the BenM EBD (residues 90-304)(FIG. 2a). Following mutagenesis we harnessed yeast's homologous recombination machinery for plasmid gap repair of variant EBDs with the BenM DNA-binding domain (DBD)(FIG. 2a). A population derived from approx. 40,000 transformants was analyzed by fluorescence activated cell sorting (FACS) using a two-step approach, in which we first removed the variants showing increased basal activity. Next, we compared fluorescence output from the population of transformants in control and CCM medium (FIG. 2b). From this, all cells showing higher fluorescence than the fluorescence observed in control medium were sorted (FIG. 2b). Sorted cells were subsequently cultivated as clones and validated by flow cytometry (FIG. 2c).

Here we identified five BenM variants with higher dynamic ranges than wild-type BenM (FIG. 2c). Sequencing of the BenM variants identified a triple mutant with point mutations H110R, F211V and Y286N in the BenM EBD (FIG. 2c). Plasmid-based expression of BenM^{H110R, F211V, Y286N} showed doubled GFP output upon CCM induction (6-fold), compared to induction for the plasmid-based expression of wild-type BenM (FIG. 2c). Interestingly, the mutations in BenM^{H110R, F211V, Y286N} were not positioned in the immediate vicinity of the CCM binding site (FIG. 2d). Similar to all other genetic devices engineered in this study, BenM^{H110R, F211V, Y286N} was also integrated into the genome for stable expression.

LTTR-Based Biosensor Specificity and Orthogonality

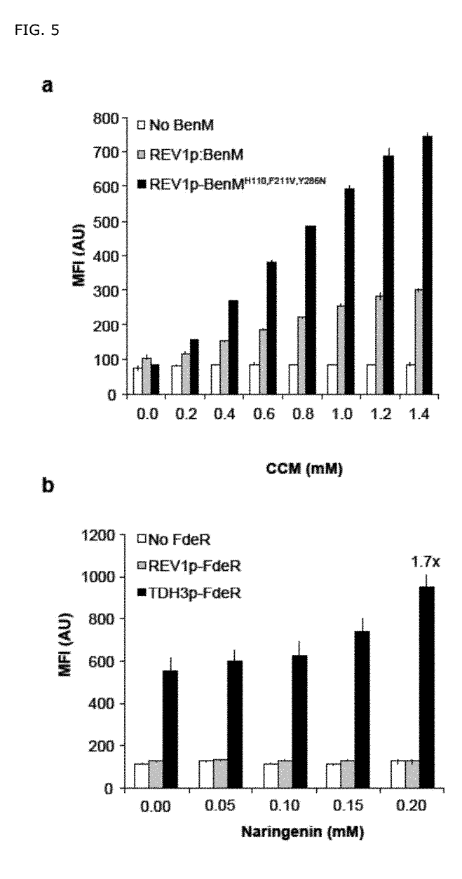

To assess the potential application of the LTTR-based biosensor for CCM in yeast, we next investigated the specificity of BenM, as well as its potential impact on the host transcriptome. First, by testing a range of diacids supplied to the growth medium at pH 4.5 with identical molar concentrations to CCM (1.4 mM), we observed that among the diacids tested both BenM and BenM^{H110R, F211V, Y286N} induce GFP expression specifically in response to CCM (FIG. 3a). Second, to test for transcriptional orthogonality of BenM^{H110R, F211V, Y286N} in yeast, we used RNA-seq to quantify and compare the transcriptomes of cells with (MeLS0284) or without (MeLS0138) expression of BenM^{H110R, F211V, Y286N}. As the genetic device has low basal activity (FIG. 4 and Table 2) we analyzed yeast transcriptomes following 24 h cultivation in the presence of CCM. Here, we observed that the average GFP transcript abundance from strain MeLS0284 was approximately 27-fold higher compared to strain MeLS0138 (FIG. 3b, FIG. 5). Apart from genes encoding GFP and BenM, only one other gene encoding the Golgi-associated retrograde protein complex component TCS3, passed our stringent cut-off (P<0.05, >2-fold) showing a modest decrease (2.3×) in expression level when BenM^{H110R, F211V, Y286N} was expressed (FIG. 3b). We found no match to BenO in this gene's promoter (data not shown), suggesting that the minor transcriptome perturbations could be due to noise in RNA-seq measurements or indirect effects.

A Design for Onboarding LTTR-Based Biosensors in Yeast

The genetic device developed in this study represents to the best of our knowledge the first example of transplanting a prokaryotic transcriptional activator into a eukaryotic chassis and successfully using it to activate gene expression without the need for modifying the protein beyond codon optimization. Acknowledging the vast numbers of transcriptional activators found among LTTR members, the optimal reporter promoter design (209 bp_CYC1p_BenO_T1) could prove valid for other metabolic engineering and biotechnological applications. To test the generality of the biosensor design for onboarding other small-molecule binding transcriptional activators as biosensors in yeast we selected four other candidates from the LTTR family; FdeR from Herbaspirillum seropedicae, PcaQ from Sinorhizobium meliloti, ArgP from Escherichia coli, and MdcR from Klebsiella pneumonia, with co-inducers naringenin, protocatechuic acid (PCA), L-arginine, and malonic acid, respectively. In this proof-of-principle study we selected the four candidates based on a minimal set of information, including knowledge about operator sequences, experimental evidence for ligand-inducible control of target operons, and their mode-of-action within native chassis (ie. activation, FIG. 1). Furthermore all of these metabolites can passively diffuse across the yeast plasma membrane, with the exception of malonic acid, which requires the expression of the dicarboxylic acid transporter MAE1 from Schizosaccharomyces pombe. For this purpose, the gene encoding MAE1 was integrated into cells expressing MdcR (Table 3). Based on this knowledge, and the aforementioned selection criteria, we directly replaced BenO located in the T1 position of the 209 bp_CYC1p promoter with operator sequences for each of these LTTRs (FIG. 4a, FIG. 2a-b, Table 4). We first tested if expression of GFP could be activated upon low and high expression of individual LTTRs. From this, it was evident that all LTTRs were able to activate GFP expression from the 209 bp_CYC1p_T1 promoter when the LTTR was expressed from the strong TDH3 promoter compared to yeast cells without expression of an LTTR (1.4×-8.1×), with BenM showing the strongest activation (8.1×)(FIG. 4a). Similarly, GFP expression could also be induced by ArgP when the weak REV1p promoter controlled expression of the LTTR (2.2×). This proves the broad applicability of the reporter promoter design, and that biosensor output is tunable depending on the expression level of the LTTR. Next, we tested if each LTTR could further induce GFP expression when its cognate inducer was supplied to the growth medium (FIG. 4b). For this purpose we prepared medium with either 1.4 mM CCM, 0.2 mM naringenin, 30 mM L-arginine, 1.4 mM PCA, or 10 mM malonic acid, as previously reported to be relevant concentrations in terms of bio-based production and microbial physiology. Here, in addition to BenM, ArgP was the only LTTR enabling a significant ligand-inducible increase in GFP expression when LTTR expression was controlled by REV1p (FIG. 4b). However, when expressing LTTRs from the TDH3 promoter all LTTRs, except PcaQ, significantly increased GFP expression (1.4×-4.1×) when their cognate ligand was present in the cultivation medium (FIG. 4b). Taken together all tested LTTRs were able to activate expression of GFP when their operators were placed in the T1 position of the 209 bp_CYC1p scaffold promoter (Table 4). Furthermore, just as for BenM, yeast expressing FdeR, ArgP and MdcR from the strong TDH3 promoter, were able to further induce GFP expression upon addition of their cognate inducers (FIG. 4b).

Many of the characterized LTTRs regulate operons by binding prototypic LTTR box patterns 5′-T-N11-A-3′ and 5′-TTA-N7/8-GAA-3′. In addition to transcriptional orthogonality (FIG. 3b), we therefore further tested if individual LTTRs would cross-react with operators for another LTTR. For this purpose, we expressed LTTRs ArgP and MdcR together with the 209 bp_CYC1p_T1 promoter with operators for MdcR (herein MdcO) or ArgP (herein ArgO) driving the expression of GFP. As controls we tested GFP expression from 209 bp_CYC1p_T1 promoter with MdcO or ArgO without expression of LTTRs. Flow cytometry analysis showed specificity between LTTR transcriptional activators and their inferred operator (FIG. 4c). This is in agreement with another study on cross-reactivity between promoter and transcriptional regulators of the TetR family, and the fact that LTTR residues in both the conserved N-terminal DNA-binding domains and the divergent EBDs are important for DNA-binding.

In Vivo Application of LTTR-Based Biosensors in Yeast

Based on our engineering efforts and characterization of prokaryote LTTR-based biosensors imported into yeast, we next addressed whether such biosensors would support real-time monitoring of product accumulation in vivo and thereby potentially provide high-throughput screening assays of biocatalysts. To test this we selected CCM and naringenin, for which highest titers in shake-flask cultivated haploid yeast of approx. 1 mM (141 mg/L) and 0.2 mM (54 mg/L), respectively, have recently been reported. Also, these two products are of general interest to biotechnology with CCM being a platform chemical for the production of several valuable consumer bio-plastics, whereas naringenin belongs to a class of secondary metabolites called flavonoids with nutritional and agricultural value.

Before applying the biosensors for in vivo detection of these metabolites we first tested their operational range and induction kinetics. For BenM and BenM^{H110R, F211V, Y286N}, we observed a weakly sigmoidal input-output relationship between CCM concentration and GFP output following 24 h cultivation. For chromosomally integrated BenM^{H110R, F211V, Y286N} and BenM, a maximum of 10- and 3.5-fold induction was reached in the presence of the highest soluble CCM concentrations (1.4 mM, 200 mg/L)(FIG. 5a). Interestingly, induction kinetics of BenM and BenM^{H110R, F211V, Y286N} were similar. This is in line with BenM mutations likely not to be involved with direct binding of CCM (FIG. 2d), but rather alter BenM binding to DNA to support increased GFP expression.

Similarly, for FdeR we first tested naringenin sensitivity and operational range of the sensor. As for CCM, the operational range was only tested for concentrations of naringenin soluble in growth medium (ie. <0.2 mM). Here, we observed that expression of FdeR controlled by the weak REV1 promoter did not support induction of GFP expression at any of the tested concentrations (FIG. 5b), yet when expression of FdeR was controlled by the strong TDH3 promoter a maximum 1.7-fold increase in GFP expression was observed following 24 h cultivation in the presence of 0.2 mM naringenin (FIG. 5b). Taken together, the operational ranges of BenM and FdeR are within the ranges of reported CCM and naringenin production titers in yeast, and therefore could make them applicable for screening such biocatalysts.

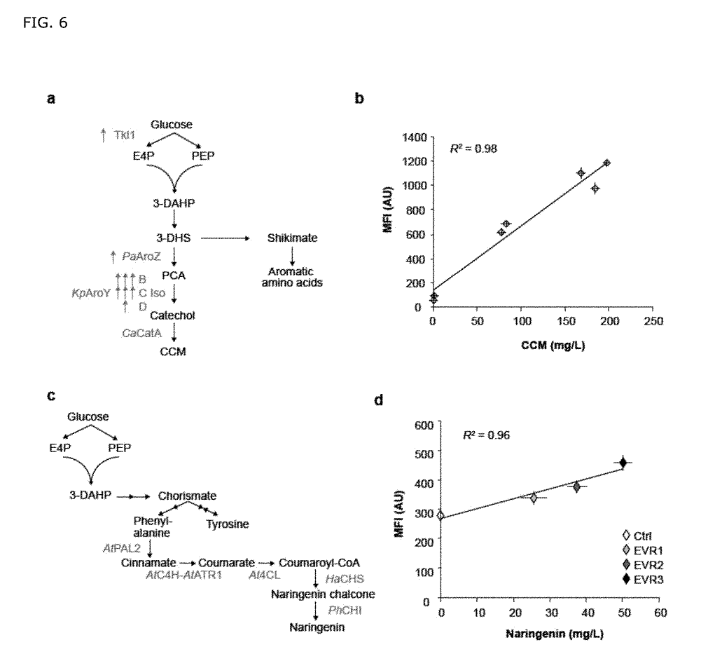

Next, we transformed the CCM biosensor (209 bp_CYC1p_BenO_T1::GFP and REV1p::BenM^{H110R, F211V, Y286N}) into a small library of six yeast strains engineered to produce CCM. CCM production with a final titer of 149 mg/L was recently reported in haploid yeast using a three-step heterologous pathway consisting of a AroZ homologue from Podospora anserina encoding dehydroshikimate dehydratase (PaAroZ), the AroY gene from Klebsiella pneumonia encoding the multi-subunit protocatechuic acid decarboxylase (PCA-DC) and the CatA gene encoding catechol 1,2-dioxygenase from Candida albicans (CaCatA) (FIG. 6a). From that study it was clear that PCA-DC was a rate-limiting step for flux through the upper part of the shikimate pathway towards CCM. It was also suggested that an increased supply of precursor towards erythrose-4-phosphate (E4P) could improve CCM production. For this reason we introduced single or multiple copies of different PCA-DC subunits from K. pneumonia and introduced no or one additional copy of transketolase (Tkl1) from S. cerevisiae (FIG. 6a). The six-membered CCM production strain library and a wild-type CCM null background strain were cultured individually. After 24 h of cultivation the medium was analyzed for CCM concentration using HPLC and the cells were analyzed by flow cytometry for GFP intensity measurements. Here, we observed a strong correlation (r=0.98) between GFP output and CCM production titers, spanning a range of 0.00016-1.39 mM (0.023-197.6 mg/L)(FIG. 6b). The highest titers were obtained in strain ST4245-2 with multiple TY integrations of AroY subunits B and C and Tkl1 (FIG. 6a-b). To further examine the performance of the CCM biosensor we monitored GFP output and CCM production titers following 72 h of cultivation. Here, GFP outputs were saturated at titers >1.41 mM (200 mg/L)(FIG. 6a-b). However, the strain that produced the most CCM after 72 h (3.03 mM, 430.8 mg/L) also produced the most CCM and had the highest fluorescence after 24 h, emphasizing the applicability of the CCM biosensor for screening high-producing strains during early stages of cultivation.

Finally, we transformed 209 bp_CYC1p_FdeO_T1::GFP and TDH3p::FdeR into yeast strains with a 5-step heterologous naringenin pathway. For building a small library of naringenin producing strains, we chromosomally introduced either in single copy of the pathway (EVR1), or with one and two additional integrations of bottleneck enzymes (AtPAL-2 and HaCHS for EVR2; AtPAL-2, HaCHS, and AtC4H:L5:AtATR2 for EVR3)(FIG. 6c, Table 1). Following 48 h of cultivation the medium was analyzed for naringenin concentration using UPLC and the cells were analyzed by flow cytometry for GFP intensity measurements. As observed for the CCM biosensor, the naringenin biosensor also had a strong correlation (r=0.96) between GFP output and naringenin titers, spanning a range of 0.094-0.184 mM (25.61-50.18 mg/L)(FIG. 6d), with the highest titer obtained in strain EVR3 containing two additional integrations of bottleneck enzymes on top of the full copy of the 5-step naringenin pathway. For the naringenin sensor we observed a poorer correlation between biosensor output and titers at 24 h (r=0.87) compared to our 48 h (r=0.96) measurements (FIG. 6c-d). However, just as for the CCM biosensor, the strain that produced the most naringenin at 48 h (0.184 mM, 50.18 mg/L) also produced the most naringenin (0.045 mM, 12.25 mg/L) and had the highest fluorescence at 24 h.

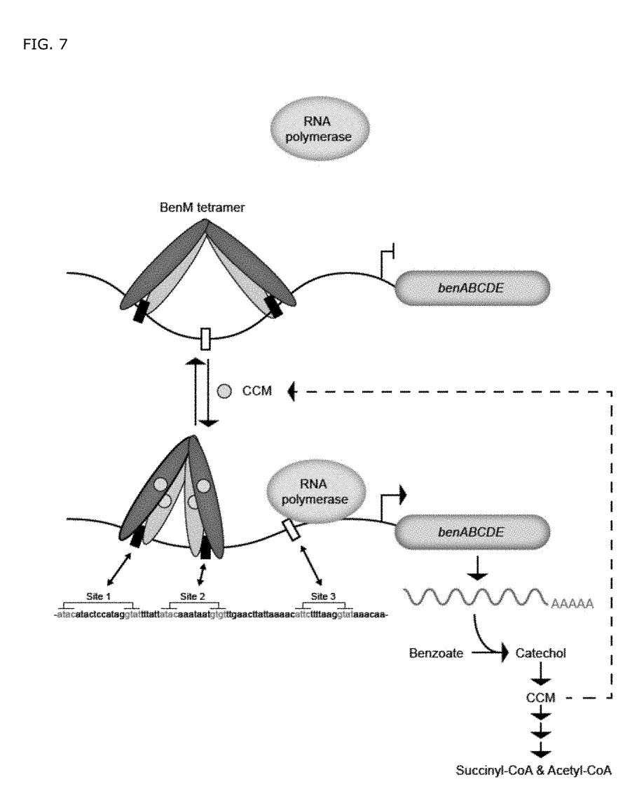

Taken together, the two applications of the LTTR-based biosensors suggest that simple expression of the LTTR and an engineered reporter promoter (209 bp_CYC1p_T1::GFP) with an operator site in position T1 allows for direct transplantation of prokaryotic transcriptional activators as biosensors to screen for the best-performing biocatalysts. Interestingly, though some of the transcriptional activators used in this study derived from prokaryotes with growth optima at higher temperatures compared to yeast, BenM showed a higher dynamic range in output at 30° C. compared to 37° C. (FIG. 7), illustrating robustness of LTTR performance.

Application of Biosensors in CHO Cells

For testing the reporter promoter design with other promoter backbones AND in another eukaryotes, Chinese hamster ovary cells was transformed using the human cytomegalovirus promoter backbone (CMV) instead of the CYC1 promoter backbone used in yeast.

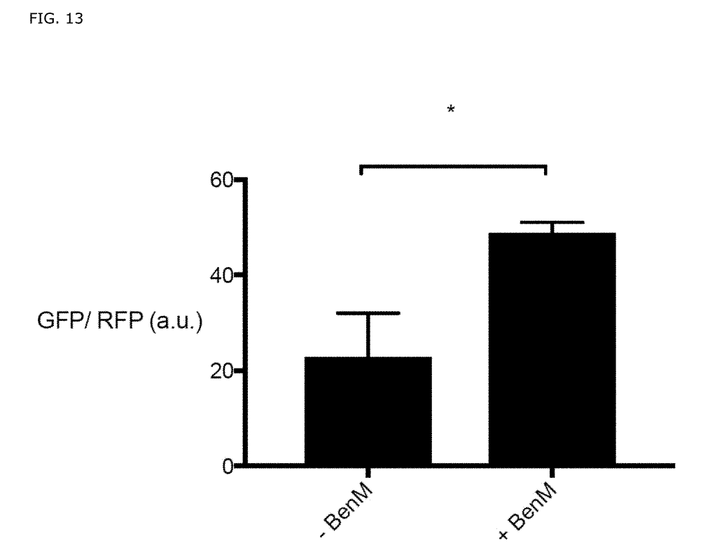

Just as in the case with the yeast design using CYC1 promoter as a backbone, the present inventors put the binding site (benO) for the prokaryotic transcriptional activator BenM 6 bp upstream of the TATA box the CMV promoter and scored reporter gene activity (GFP fluorescence) in the presence and absence of the transcriptional activator BenM. As can be seen in FIG. 13, the design worked when putting into another promoter backbone AND another host organism (CHO cells).

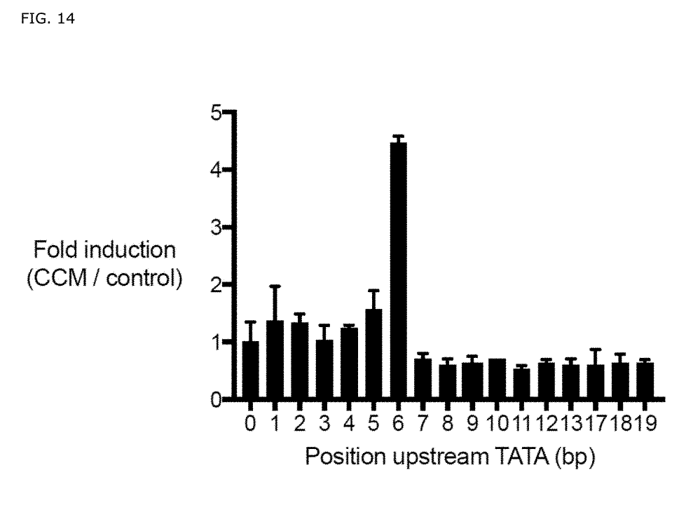

In addition to this, the present inventors also tested 17 different positions for positioning of the BenM binding site (benO). Only position 6 upstream of the TATA box gave a significant response (See FIG. 14).

Prokaryotic operator benO for the prokaryotic transcriptional activator BenM is placed 6 bp upstream of the TATA box the CMV promoter

| | |
|---|---|
| 1 | GTTGACATTG ATTATTGACT AGTTATTAAT AGTAATCAAT TACGGGGTCA |
| 51 | TTAGTTCATA GCCCATATAT GGAGTTCCGC GTTACATAAC TTACGGTAAA |
| 101 | TGGCCCGCCT GGCTGACCGC CCAACGACCC CCGCCCATTG ACGTCAATAA |
| 151 | TGACGTATGT TCCCATAGTA ACGCCAATAG GGACTTTCCA TTGACGTCAA |
| 201 | TGGGTGGAGT ATTTACGGTA AACTGCCCAC TTGGCAGTAC ATCAAGTGTA |
| 251 | TCATATGCCA AGTACGCCCC CTATTGACGT CAATGACGGT AAATGGCCCG |
| 301 | CCTGGCATTA TGCCCAGTAC ATGACCTTAT GGGACTTTCC TACTTGGCAG |
| 351 | TACATCTACG TATTAGTCAT CGCTATTACC ATGGTGATGC GGTTTTGGCA |
| 401 | GTACATCAAT GGGCGTGGAT AGCGGTTTGA CTCACGGGGA TTTCCAAGTC |
| 451 | TCCACCCCAT TGACGTCAAT GGGAGTTTGT TTTGGCACCA AAATCAACGG |
| 501 | GACTTTCCAA AATGTCGTAA CAACTCCGCC CCATTGACGC AAATGGGCGG |
| 551 | TAGGCGTGTA CGGTGGATAC TCCATAGGTA TTTTATTATA CAAATAATGT |
| 601 | GTTTGAACTT ATTAAAACAT TCTTTTAAGG TATAAACAAG AGGTC [symbol] T |
| 651 | AAGCAGAGCT C [symbol] |

BenO (Bold) [symbol]
(Bold, Italic) [symbol]
(BoldfItalic, underlined)
START CODON (underlined)

### LIST OF REFERENCES

1. Jakočiūnas, T., Jensen, M. K. & Keasling, J. D. CRISPR/Cas9 advances engineering of microbial cell factories. Metab. Eng. 34, 44-59 (2015).
2. Esvelt, K. M. & Wang, H. H. Genome-scale engineering for systems and synthetic biology. Mol. Syst. Biol. 9, 641 (2013).
3. Elowitz, M. B. & Leibler, S. A synthetic oscillatory network of transcriptional regulators. Nature 403, 335-8 (2000).
4. Wang, B., Barahona, M. & Buck, M. Amplification of small molecule-inducible gene expression via tuning of intracellular receptor densities. Nucleic Acids Res. 43, 1955-64 (2015).
5. Farzadfard, F. & Lu, T. K. Genomically encoded analog memory with precise in vivo DNA writing in living cell populations. Science (80-.). 346, 1256272-1256272 (2014).
6. Michener, J. K. & Smolke, C. D. High-throughput enzyme evolution in Saccharomyces cerevisiae using a synthetic RNA switch. Metab. Eng. 14, 306-16 (2012).
7. Raman, S., Rogers, J. K., Taylor, N. D. & Church, G. M. Evolution-guided optimization of biosynthetic pathways. Proc. Natl. Acad. Sci. 111, 201409523 (2014).
8. Choi, J. H. & Ostermeier, M. Rational design of a fusion protein to exhibit disulfide-mediated logic gate behavior. ACS Synth. Biol. 4, 400-6 (2015).
9. Auslander, S., Auslander, D., Müller, M., Wieland, M. & Fussenegger, M. Programmable single-cell mammalian biocomputers. Nature 487, 123-127 (2012).
10. Khalil, A. S. et al. A synthetic biology framework for programming eukaryotic transcription functions. Cell 150, 647-58 (2012).
11. Folcher, M., Xie, M., Spinnler, A. & Fussenegger, M. Synthetic mammalian trigger-controlled bipartite transcription factors. Nucleic Acids Res. 41, e134 (2013).
12. Stanton, B. C. et al. Genomic mining of prokaryotic repressors for orthogonal logic gates. Nat. Chem. Biol. 10, 99-105 (2014).
13. Gossen, M. & Bujard, H. Tight control of gene expression in mammalian cells by tetracycline-responsive promoters. Proc. Natl. Acad. Sci. U.S.A 89, 5547-51 (1992).
14. Stanton, B. C. et al. Systematic transfer of prokaryotic sensors and circuits to mammalian cells. ACS Synth. Biol. 3, 880-91 (2014).
15. Gilbert, L. A. et al. CRISPR-mediated modular RNA-guided regulation of transcription in eukaryotes. Cell 154, 442-51 (2013).
16. Gossen, M. et al. Transcriptional activation by tetracyclines in mammalian cells. Science 268, 1766-9 (1995).
17. Teo, W. S. & Chang, M. W. Bacterial XylRs and synthetic promoters function as genetically encoded xylose biosensors in Saccharomyces cerevisiae. Biotechnol. J. 10, 315-22 (2015).
18. Lee, N., Francklyn, C. & Hamilton, E. P. Arabinose-induced binding of AraC protein to aral2 activates the araBAD operon promoter. Proc. Natl. Acad. Sci. U.S.A 84, 8814-8 (1987).
19. Shadel, G. S. & Baldwin, T. O. The Vibrio fischeri LuxR protein is capable of bidirectional stimulation of transcription and both positive and negative regulation of the luxR gene. J. Bacteriol. 173, 568-74 (1991).
20. Lee, D. J., Minchin, S. D. & Busby, S. J. W. Activating Transcription in Bacteria. Annu. Rev. Microbiol. 66, 125-52 (2012).
21. Siedler, S., Stahlhut, S. G., Malla, S., Maury, J. & Neves, A. R. Novel biosensors based on flavonoid-responsive transcriptional regulators introduced into Escherichia coli. Metab. Eng. 21, 2-8 (2014).
22. Maddocks, S. E. & Oyston, P. C. F. Structure and function of the LysR-type transcriptional regulator (LTTR) family proteins. Microbiology 154, 3609-23 (2008).
23. Collier, L. S., Gaines, G. L. & Neidle, E. L. Regulation of benzoate degradation in Acinetobacter sp. strain ADP1 by BenM, a LysR-type transcriptional activator. J. Bacteriol. 180, 2493-501 (1998).
24. Suastegui, M. et al. Combining Metabolic Engineering and Electrocatalysis: Application to the Production of Polyamides from Sugar. Angew. Chemie 128, 2414-2419 (2016).
25. Curran, K. A., Leavitt, J. M., Karim, A. S. & Alper, H. S. Metabolic engineering of muconic acid production in Saccharomyces cerevisiae. Metab. Eng. 15, 55-66 (2013).
26. Bundy, B. M., Collier, L. S., Hoover, T. R. & Neidle, E. L. Synergistic transcriptional activation by one regulatory protein in response to two metabolites. Proc. Natl. Acad. Sci. U.S.A 99, 7693-8 (2002).
27. Wang, M., Li, S. & Zhao, H. Design and engineering of intracellular-metabolite-sensing/regulation gene circuits in Saccharomyces cerevisiae. Biotechnol. Bioeng. 113, 206-15 (2016).
28. Olesen, J., Hahn, S. & Guarente, L. Yeast HAP2 and HAP3 activators both bind to the CYC1 upstream activation site, UAS2, in an interdependent manner. Cell 51, 953-61 (1987).
29. McIsaac, R. S., Gibney, P. A., Chandran, S. S., Benjamin, K. R. & Botstein, D. Synthetic biology tools for programming gene expression without nutritional perturbations in Saccharomyces cerevisiae. Nucleic Acids Res. (2014). doi:10.1093/nar/gkt1402
30. Li, W. Z. & Sherman, F. Two types of TATA elements for the CYC1 gene of the yeast Saccharomyces cerevisiae. Mol. Cell. Biol. 11, 666-76 (1991).
31. Pfeifer, K., Arcangioli, B. & Guarente, L. Yeast HAP1 activator competes with the factor RC2 for binding to the upstream activation site UAS1 of the CYC1 gene. Cell 49, 9-18 (1987).
32. Lee, M. E., Aswani, A., Han, A. S., Tomlin, C. J. & Dueber, J. E. Expression-level optimization of a multi-enzyme pathway in the absence of a high-throughput assay. Nucleic Acids Res. 41, 10668-78 (2013).
33. Peng, H. L., Shiou, S. R. & Chang, H. Y. Characterization of mdcR, a regulatory gene of the malonate catabolic system in Klebsiella pneumoniae. J. Bacteriol. 181, 2302-6 (1999).
34. MacLean, A. M., MacPherson, G., Aneja, P. & Finan, T. M. Characterization of the beta-ketoadipate pathway in Sinorhizobium meliloti. Appl. Environ. Microbiol. 72, 5403-13 (2006).
35. Laishram, R. S. & Gowrishankar, J. Environmental regulation operating at the promoter clearance step of bacterial transcription. Genes Dev. 21, 1258-72 (2007).
36. Maclean, A. M., Haerty, W., Golding, G. B. & Finan, T. M. The LysR-type PcaQ protein regulates expression of a protocatechuate-inducible ABC-type transport system in Sinorhizobium meliloti. Microbiology 157, 2522-33 (2011).
37. Chen, W. N. & Tan, K. Y. ‘Malonate uptake and metabolism in Saccharomyces cerevisiae’. Appl. Biochem. Biotechnol. 171, 44-62 (2013).
38. Opekarová, M. & Kubin, J. On the unidirectionality of arginine uptake in the yeast Saccharomyces cerevisiae. FEMS Microbiol. Lett. 152, 261-7 (1997).
39. Rogers, J. K. & Church, G. M. Genetically encoded sensors enable real-time observation of metabolite production. Proc. Natl. Acad. Sci. U.S.A (2016). doi:10.1073/pnas.1600375113
40. Rikhvanov, E. G., Varakina, N. N., Rusaleva, T. M., Rachenko, E. I. & Voinikov, V. K. [The effect of sodium malonate on yeast thermotolerance]. Mikrobiologiia 72, 616-20
41. Koopman, F. et al. De novo production of the flavonoid naringenin in engineered Saccharomyces cerevisiae. Microb. Cell Fact. 11, 155 (2012).
42. Winkel-Shirley, B. Flavonoid biosynthesis. A colorful model for genetics, biochemistry, cell biology, and biotechnology. Plant Physiol. 126, 485-93 (2001).
43. Naesby, M. et al. Yeast artificial chromosomes employed for random assembly of biosynthetic pathways and production of diverse compounds in Saccharomyces cerevisiae. Microb. Cell Fact. 8, 45 (2009).
44. Gupta, R. K., Patterson, S. S., Ripp, S., Simpson, M. L. & Sayler, G. S. Expression of the Photorhabdus luminescens lux genes (luxA, B, C, D, and E) in Saccharomyces cerevisiae. FEMS Yeast Res. 4, 305-13 (2003).
45. Galloway, K. E., Franco, E. & Smolke, C. D. Dynamically reshaping signaling networks to program cell fate via genetic controllers. Science 341, 1235005 (2013).
46. Kim, T., Folcher, M., Doaud-El Baba, M. & Fussenegger, M. A synthetic erectile optogenetic stimulator enabling blue-light-inducible penile erection. Angew. Chem. Int. Ed. Engl. 54, 5933-8 (2015).
47. Zhang, H., Li, Z., Pereira, B. & Stephanopoulos, G. Engineering E. coli-E. coli cocultures for production of muconic acid from glycerol. Microb. Cell Fact. 14, 134 (2015).
48. Trapnell, C. et al. Differential gene and transcript expression analysis of RNA-seq experiments with TopHat and Cufflinks. Nat. Protoc. 7, 562-78 (2012).
49. Jensen, N. B. et al. EasyClone: method for iterative chromosomal integration of multiple genes in Saccharomyces cerevisiae. FEMS Yeast Res. 14, 238-48 (2014).
50. Gietz, R. D. & Schiestl, R. H. Large-scale high-efficiency yeast transformation using the LiAc/SS carrier DNA/PEG method. Nat. Protoc. 2, 38-41 (2007).
51. Eckert-Boulet, N., Pedersen, M. L., Krogh, B. O. & Lisby, M. Optimization of ordered plasmid assembly by gap repair in Saccharomyces cerevisiae. Yeast 29, 323-34 (2012).
52. Mikkelsen, M. D. et al. Microbial production of indolylglucosinolate through engineering of a multi-gene pathway in a versatile yeast expression platform. Metab. Eng. 14, 104-11 (2012).
53. Kildegaard, K. R. et al. Evolution reveals a glutathione-dependent mechanism of 3-hydroxypropionic acid tolerance. Metab. Eng. 26, 57-66 (2014).
54. Pettersen, E. F. et al. UCSF Chimera—a visualization system for exploratory research and analysis. J. Comput. Chem. 25, 1605-12 (2004).

1-26. (canceled)

27. A eukaryotic cell, such as a yeast cell comprising a bacterial transcriptional activator and a corresponding operator sequence positioned in a eukaryotic promoter, such as positioned within an endogenous promoter of said cell, which activator controls the expression of a gene from said eukaryotic promoter.

28. The cell according to claim 27, wherein the expression of a gene from said eukaryotic promoter is depending on the presence, such as dose dependent, of a ligand specifically binding said transcriptional activator.

29. The cell according to claim 28, wherein said cell comprises a gene encoding the expression of said ligand, one or more genes encoding a pathway of enzymes synthesizing said ligand, and/or a gene encoding a compound that is metabolized into said ligand.

30. The cell according to claim 27, wherein said cell comprises an exogenous reporter gene, and/or one or more further regulatory gene, such as a gene encoding antibiotic resistance, or a reporter gene that provides for fluorescence output, such as a gene encoding green fluorescent protein, blue fluorescent protein or luciferase.

31. The cell according to claim 28, wherein one or more of the genes independently selected from the gene encoding the expression of said ligand, one or more genes encoding a pathway of enzymes synthesizing said ligand, a gene encoding a compound that is metabolized into said ligand, an exogenous reporter gene, and one or more further regulatory gene; is under the control and/or is activated by said eukaryotic promoter.

32. The cell according to claim 27, wherein said transcriptional activator is selected from any one selected from table 6, such as any one selected from BenM, FdeR, MdcR, and ArgP.

33. The cell according to claim 28, wherein said ligand and transcriptional activator is any one pair selected from table 7, such as any one selected from muconic acid and BenM; Naringenin and FdeR; Malonate and MdcR; and L-arginin and ArgP.

34. The cell according to claim 27, which is a yeast cell, such as Saccharomyces cerevisiae, or a mammalian cell, such as a Chinese hamster ovary cell.

35. The cell according to claim 27, wherein said promoter is a full length promoter, or a truncated version with upstream activating sequences, such as UAS1 and UAS2 of the CYC promoter, removed.

36. The cell according to claim 27, wherein said transcriptional activator does not require binding to any other regulatory subunits and/or which cell is without any further engineering or the co-expression of other molecular components regulating said transcriptional activator and/or wherein said transcriptional activator does not require binding to any other regulatory subunits apart from its specific ligand and/or which cell is without any further engineering or the co-expression of other molecular components regulating said transcriptional activator.

37. The cell according to claim 27, which operator sequence is specific for said transcriptional activator within said promoter.

38. The cell according to claim 37, wherein said operator sequence is positioned immediately 6 bp upstream of the TATA box, such as a TATA box 1, such as TATA-113, such as anywhere between 6-15 bp, such as anywhere between 6-14 bp, such as anywhere between 6-13 bp, such as anywhere between 6-12 bp; such as anywhere between 6-11 bp, such as anywhere between 6-10 bp, such as anywhere between 6-9 bp, such as anywhere between 6-8 bp, such as anywhere between 6-7 bp, such as 6 bp upstream of said TATA box of said eukaryotic promoter.

39. Use of a prokaryotic transcriptional activator as a regulator of transcription in a eukaryotic cell, such as a yeast cell according to claim 27, said transcriptional activator being activated by a ligand specifically binding said transcriptional activator to induce the expression of a protein product from a eukaryotic promoter of said cell, said promoter containing the operator sequence corresponding to said transcriptional activator.

40. Use of a prokaryotic transcriptional activator as a metabolite biosensor for measuring the amount of a ligand extracellular of and/or produced by a eukaryotic cell, such as a yeast cell according to claim 27, wherein said ligand specifically bind said transcriptional activator to induce expression of a reporter gene from a eukaryotic promoter of said cell, said promoter containing the operator sequence corresponding to said transcriptional activator.

41. Method for measuring the amount of a ligand intracellular or extracellular of a eukaryotic cell, such as a yeast cell; said cell comprising a bacterial transcriptional activator and a corresponding operator sequence, which activator controls the expression of a reporter gene from a eukaryotic promoter of said cell in response to said ligand specifically binding said transcriptional activator; said promoter containing the operator sequence corresponding to said transcriptional activator; said method including the steps of
a) Cultivating a eukaryotic cell as defined in claim 27;
b) Measuring the output from said promoter of said reporter gene;
c) Correlating said output from step b) with amount of said ligand.
